(19) United States
(12) Patent Application Publication
(10) Pub. No.: US 2004/0009915 A1
(43) Pub. Date: Jan. 15, 2004

(54) Polynucleotides encoding a novel intracellular chloride channel-related polypeptide

(76) Inventors: Han Chang, Princeton Junction, NJ (US); John N. Feder, Belle Mead, NJ (US); Liana M. Lee, Somerset, NJ (US); Adam Rich, Yardley, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/384,919

(22) Filed: Mar. 6, 2003

Related U.S. Application Data
(60) Provisional application No. 60/362,257, filed on Mar. 6, 2002.

(51) Int. Cl. C07K 14/705; C12P 21/02; C12N 5/06; C07K 16/28; A61K 38/17

(52) U.S. Cl. 514/12; 530/350; 530/388.1; 435/69.1; 435/320.1; 435/325

(57) ABSTRACT

The present invention describes the novel human intracellular chloride ion channel-related protein HCLI and its encoding polynucleotide. Also described are expression vectors, host cells, antisense molecules, and antibodies associated with the HCLI polynucleotide and/or polypeptide of this invention. In addition, methods for treating, diagnosing, preventing, and screening for disorders or diseases associated with abnormal biological activity of HCLI are described, as are methods for screening for modulators, e.g., agonists or antagonists, of HCLI activity and/or function.

This application is a continuation-in-part application of provisional application U.S. Ser. No. 60/362,257 filed Mar. 6, 2002, and claims benefit of the same under 35 U.S.C. 119(e). The entire teachings of the referenced application are incorporated herein by reference.

### FIELD OF THE INVENTION

The present invention relates to a novel intracellular chloride ion channel polynucleotide, called HCLI herein, and its nucleic acid (polynucleotide) sequence which encodes an HCLI protein, of the chloride channel family. This invention further relates to fragments of the HCLI nucleic acid sequence and its encoded amino acid sequence. Additionally, the invention relates to methods of using the HCLI polynucleotide sequence and encoded HCLI protein for diagnosis, polynucleotide screening and for the treatment of diseases, disorders, conditions, or syndromes associated with HCLI.

### BACKGROUND OF THE INVENTION

Ion channels are ubiquitous transmembrane proteins that confer selective ionic permeability to cell surface and intracellular membranes in virtually every cell in every known organism. Research on ion channels during the past sixty years has focused predominantly on proteins that mediate selective permeability to monovalent (Na⁺, K⁺) and divalent (Ca²⁺) cations. But recently, there has been intense interest with ion channels that are selectively permeable to chloride (Cl⁻) ions, as their importance in human diseases and fundamental cellular events has been elucidated.

The ClC-type Cl⁻ channels, make up a single protein family (Jentsch, T. et al., Nature, 348:510-514, (1990); Jentsch, T., Curr. Opin. Neurobiol., 6:303-310, (1996); Maduke, M. et al., Annu. Rev. Biophys. Biomol. Struct., 29:411-438, (2000); Jetsch, T. et al., Pflugers Arch., 437:783-795, (1999)). ClC-type channels are completely unrelated in sequence to the known cation channels, or to other known anion-conducting channels, including the cystic fibrosis transmembrane conductance regulator (CFTR) chloride channels, porins, and γ-aminobutyric acid (GABA) receptors. The ClC family is evolutionarily ancient, with members described in all living kingdoms. Broad sequence identity among ClC homologs is limited to a few highly conserved stretches of amino acids, i.e., ‘hot spots,’ distributed throughout the protein. Overall sequence identity between family members from different kingdoms is about 15-20%, but in the hot spots, sequence identity is much higher, with nearly identical amino-acid sequences found in widely divergent species. Overall patterns of hydrophobicity are also strongly conserved in all known ClCs.

Currently, the mammalian ClC chloride channel family contains nine known members (Jentsch, T., Curr. Opin. Neurobiol., 6:303-310, (1996)). These nine members are divided into three ClC subfamilies: (1) ClC-0, ClC-1, ClC-2, ClC-Ka (ClCK1), ClC-Kb (ClCK2); (2) ClC-3, ClC-4, ClC-5; and (3) ClC-6, ClC-7. The encoded channel proteins within each subfamily are quite closely related, with protein sequence identities in the range of 50-80%. In contrast, sequence identity between subfamilies is almost as low as that between ClCs from different kingdoms (about 20%), suggesting that the subfamilies diverged early in the history of the animals (Mindell, J. et al., Genome Biol., 2(2):3003.1-3003.6, (2001)).

Structurally, the ClC channels are unique as they are homodimers with a two-fold axis perpendicular to the membrane plane, where each of the subunits within the dimer forms its own ion-conduction pore (Middleton, R. et al., Nature, 383:337-340, (1996); Ludewig, U. et al., Nature, 383:340-343, (1996); Dutzler, R. et al., Nature, 415:287-294, (2002)). In contrast, all other known α-helical ion channel proteins form one-pore oligomers of four-, five- or six-fold symmetry, where the pore occurs at the axis of symmetry of the oligomer. The ClC Cl⁻ channel subunit contains 18 α-helices that are predicted to traverse the membrane 10-12 times. Both the amino- and carboxy-terminal domains are cytoplasmic (Purdy et al., FEBS Lett., 466:26-28, (2000)).

Chloride channels (ClCs) are found in the membranes of almost every cell type, where they play a variety of roles (Franciolini, F et al., Biochim. Biophy. Acta, 1031:247-259, (1990)). Much of the data on the physiological function of ClC channels comes from studies of human polynucleotide diseases. ClC-1, which is largely expressed in skeletal muscle, is mutated in inherited myotonias in humans, goats, and mice (Koch, M. et al., Science, 257:797-800, (1992)). This association was critical in establishing ClC-1 as the major ion channel involved in setting and restoring the resting membrane voltage of skeletal muscle. Identification of ClC-Kb mutations as a cause of Bartter's syndrome, an inherited salt-wasting nephropathy, demonstrated that this channel is a critical component of the urinary concentrating mechanism of the kidney (Simon, D. et al., Nat. Genet., 17:171-178, (1997)). ClC-Ka has also been suggested to play a role in urinary concentration, as knock-out mice have nephrogenic diabetes insipidus (Matsumura, Y. et al., Nat. Genet., 21:95-98, (1999)). In Dent's disease, a pleiomorphic disorder of renal solute re-uptake caused by inherited mutations in ClC-5, patients suffer from defective endocytosis in the renal proximal tubule (Piwon, N. et al., Nature, 408:369-373 (2000)). ClC-5 and ClC-4 are mostly localized to intracellular compartment membranes. These observations support a hypothesis in which ClC-5 serves as a Cl⁻ shunt in endocytic vesicles, allowing acidification without prohibitive charge separation across the vesicular membrane.

Although the physiological function of many other ClCs remains unknown, several do have proposed functions. ClC-2, which is expressed in all mammalian tissues, has been proposed to play a role in the cellular response to volume/osmotic stimuli (Grunder, S. et al., Nature, 360:759-762, (1992)). In addition, this channel has been co-opted in some neurons to modulate their electrical excitability (Staley, K. et al., Neuron, 17:543-551, (1996)). Surprisingly, polynucleotide-knockout studies of ClC-3 had no effect on volume-regulated Cl⁻ currents, and more surprisingly, the knockout mice suffered complete depolynucleotideration of their hippocampi (Stobrawa, S. et al., Neuron, 29:185-196, (2001)). The ClC-3 channel localizes to intracellular vesicles in the hippocampus, and its inactivation impaired acidification of synaptic vesicles. Similarly, mutant mice and humans lacking ClC-7 display defects in pH regulation; this mutation causes severe osteopetrosis, a disease that results in brittle, breakable bones, due to a defect in the Cl⁻ shunt in the bone remodeling osteoclast cells (Kornak, U. et al., Cell, 104:205-215, (2001)). The emerging theme from the knockout studies is that ClCs in the ClC-3/4/5 and ClC-6/7 subfamilies play important roles in regulating pH in intracellular compartments.

All ClCs that have been studied form pores that are selective for chloride ions over large organic anions. The molecular details of the ClC channel pore remains largely unknown. However, the X-ray structures of two prokaryotic ClC channels indicate that important amino acids from four separate regions are brought together near the membrane center to form an ion-binding site (Dutzler, R. et al., Nature, 415:287-294, (2002)). These four regions are highly conserved in ClC channels, the four regions include the sequences GSGIP, G(K/R)EGP, GXFXP and Y445, respectively. It is significant that these sequences occur at the N termini of α-helices, because the resultant arrangement of the helices creates an N-terminal positive end charge, postulated to create a favorable electrostatic environment for anion binding.

The nine mammalian chloride channels described above do not constitute the entire chloride channel family, as novel chloride channels have been reported. For example, a 120 kDa phosphoprotein was previously reported to translocate from the cytosol to the apical membrane of gastric parietal cells in association with stimulation of acid (HCl) secretion (Urushidani, T. et al., J. Membr. Biol., 168:209-220, (1999)). To determine the molecular identity of the protein, its expression pattern in different tissues was studied followed by cloning of the corresponding cDNA (Nishizawa, T. et al., J. Biol. Chem., 275:11164-11173, (2000)). Immunoblot analysis showed that the 120 kDa phosphoprotein was highly enriched in tissues that secrete water, such as parietal cells, choroid plexus, salivary duct, lacrimal gland, kidney, and airway epithelial tissues. This protein was named “Parchorin” based on its highest enrichment in parietal cells and choroid plexus.

The cDNA for Parchorin from rabbit choroid plexus was isolated, and was found to encode for a protein of 637 amino acids with a predicted molecular mass of 65 kDa (Nishizawa, T. et al., J. Biol. Chem., 275(15):11164-11173 (2000)). The discrepancy between the predicted and observed molecular mass is due to Parchorin's highly acidic nature. Parchorin is a novel protein that has significant homlogy to the family of intracellular chloride channels, especially to the chloride p64 channel from bovine kidney (Redhead, C. et al., Proc. Natl. Acad. Sci. USA, 89:3716-3720, (1992)). When Parchorin is expressed as a fusion protein with green fluorescent protein (GFP), GFP-Parchorin, unlike other Chloride Intracellular Channels (CLICs), localized mainly to the cytosol. Thus Parchorin is likely to play a role in water-secreting cells, possibly through the regulation of chloride ion transport (Urushidani, T. et al., J. Membr. Biol., 168:209-220, (1999)).

Further identification of novel chloride channels is important to provide new drug targets and reagents for ion channel-related diseases and disorders.

### SUMMARY OF THE INVENTION

The present invention provides a novel member of the human chloride channel family, HCLI, and an HCLI variant. Based on sequence homology, the protein HCLI has been determined to be related to the intracellular chloride ion channel class of proteins. In particular, HCLI of this invention is most similar to the intracellular chloride channel-related protein Parchorin.

The present invention provides the HCLI polynucleotide, preferably full-length, and its encoded polypeptide. The HCLI polynucleotide and polypeptide, may be involved in a variety of diseases, disorders and conditions associated with chloride ion channel activity, which include, but are not limited to, Myotonia congenita, retinal depolynucleotideration, male infertility, neurodepolynucleotideration, Dent's disease, X-linked nephrolithiasis syndromes, infantile malignant osteopetrosis, nephrogenic diabetes insipidus, and Bartter's syndrome.

More specifically, the present invention is concerned with modulation of the HCLI polynucleotide and encoded products, particularly in providing treatments and therapies for relevant diseases. Antagonizing or inhibiting the action of the HCLI polynucleotide and polypeptide is especially encompassed by the present invention.

It is another aspect of this invention to provide the isolated HCLI polynucleotide as depicted in SEQ ID NO: 1. Also provided is the HCLI polypeptide, encoded by the polynucleotide of SEQ ID NO: 1 and having the encoded amino acid sequence of SEQ ID NO: 2, or a functional or biologically active portion of this sequence.

It is yet another aspect of the invention to provide the isolated HCLI variant polynucleotide, HCLI.v1, as depicted in SEQ ID NO: 3. Also provided is the HCLI variant polypeptide, encoded by the polynucleotide of SEQ ID NO: 3 and having the encoded amino acid sequence of SEQ ID NO: 4, or a functional or biologically active portion of this sequence.

It is yet another aspect of the invention to provide the isolated HCLI variant polynucleotide, HCLI.v2, as depicted in SEQ ID NO: 16. Also provided is the HCLI variant polypeptide, encoded by the polynucleotide of SEQ ID NO: 16 and having the encoded amino acid sequence of SEQ ID NO: 17, or a functional or biologically active portion of this sequence.

Another aspect of the invention to provide compositions comprising the HCLI polynucleotide sequence, or fragments or portions thereof, or the encoded HCLI polypeptide, or fragments or portions thereof.

Yet another aspect of the invention is to provide compositions comprising N-terminal, C-terminal or internal deletion polypeptides of the encoded HCLI polypeptide. Polynucleotides encoding these deletion polypeptides are also provided. The present invention also provides the use of these polypeptides as an immunogenic and/or antigenic epitope as described elsewhere herein.

A further aspect of this invention is to provide the polynucleotide sequence comprising the complement of SEQ ID NO: 1, or variants thereof. In addition, an aspect of the invention encompasses variations or modifications of the HCLI sequence which are the result of depolynucleotideracy of the polynucleotide code, where the polynucleotide sequences can hybridize under moderate or high stringency conditions to the polynucleotide sequence of SEQ ID NO: 1.

Another aspect of the invention is to provide the polynucleotide sequence of HCLI (SEQ ID NO: 1) lacking the initiating codon as well as the resulting encoded polypeptide. Specifically, the present invention provides the polynucleotide corresponding to nucleotides 4 through 1887 of SEQ ID NO: 1, and the polypeptide corresponding to amino acids 2 through 629 of SEQ ID NO: 2. Also provided by the present invention are recombinant vectors comprising said encoding sequence, and host cells comprising said vector.

It is another aspect of the invention to provide an antisense of the HCLI nucleic acid sequence, as well as oligonucleotides, fragments, or portions of the nucleic acid molecules or antisense molecules. Also provided are expression vectors and host cells comprising polynucleotides that encode the HCLI polypeptide.

In yet another of its aspects, the present invention provides pharmaceutical compositions comprising the HCLI polynucleotide sequence, or fragments thereof, or the encoded HCLI polypeptide sequence, or fragments or portions thereof. Also provided are pharmaceutical compositions comprising the HCLI polypeptide sequence, homologues, or one or more functional portions thereof, wherein the compositions further comprise a pharmaceutically- and/or physiologically-acceptable carrier, excipient, or diluent. All fragments or portions of the HCLI polynucleotide and polypeptide are preferably functional or active.

Another aspect of the invention is to provide methods for producing a polypeptide comprising the amino acid sequence of SEQ ID NO: 2, or a fragment thereof, preferably, a functional fragment or portion thereof, comprising the steps of a) cultivating a host cell containing an expression vector containing at least a functional fragment of the polynucleotide sequence encoding the HCLI protein according to this invention, under conditions suitable for the expression of the polypeptide; and b) recovering the polypeptide from the host cell or lysate thereof.

Another aspect of this invention is to provide a substantially purified modulator, preferably an antagonist or inhibitor, of the HCLI polypeptide having SEQ ID NO: 2. In this regard, and by way of example, a purified antibody, or binding portion thereof that binds to a polypeptide comprising the amino acid sequence of SEQ ID NO: 2 or antigenic epitope thereof, or homologue encoded by a polynucleotide having homology to the nucleic acid sequence, or depolynucleotiderate thereof, as set forth in SEQ ID NO: 1 is provided.

It is another aspect of the present invention to provide modulators of the HCLI protein and HCLI peptide targets which can affect the function or activity of HCLI in a cell in which HCLI function or activity is to be modulated or affected. In addition, modulators of HCLI can affect downstream systems and molecules that are regulated by, or which interact with, HCLI in the cell. Modulators of HCLI include compounds, materials, agents, drugs, and the like, that antagonize, inhibit, reduce, block, suppress, diminish, decrease, or eliminate HCLI function and/or activity. Such compounds, materials, agents, drugs and the like can be collectively termed “antagonists”. Alternatively, modulators of HCLI include compounds, materials, agents, drugs, and the like, that agonize, enhance, increase, augment, or amplify HCLI function in a cell. Such compounds, materials, agents, drugs and the like can be collectively termed “agonists”.

It is yet another aspect of the present invention to provide HCLI nucleic acid sequences, polypeptides, peptides and antibodies for use in the diagnosis and/or screening of disorders or diseases associated with expression of the HCLI polynucleotide and its encoded polypeptide product as described herein.

Another aspect of this invention is to provide diagnostic probes or primers for detecting HCLI-related diseases and/or for monitoring a patient's response to therapy. The probe or primer sequences comprise nucleic acid or amino acid sequences of HCLI described herein.

It is another aspect of the present invention to provide a method for detecting a polynucleotide that encodes the HCLI polypeptide in a biological sample comprising the steps of: a) hybridizing the complement of the polynucleotide sequence encoding SEQ ID NO: 1 or a hybridizable portion thereof, to the nucleic acid material of a biological sample, thereby forming a hybridization complex; and b) detecting the hybridization complex, wherein the presence of the complex correlates with the presence of a polynucleotide encoding an HCLI polypeptide in the biological sample. The nucleic acid material may be further amplified by the polymerase chain reaction (PCR) prior to hybridization, as known and practiced in the art.

Another aspect of this invention is to provide methods for screening for agents which modulate the HCLI polypeptide, e.g., agonists (or enhancers or activators) and antagonists (or blockers or inhibitors), particularly those that are obtained from the screening methods as described.

As yet a further aspect, the present invention provides methods for detecting polynucleotide predisposition, susceptibility and/or response to therapy of various HCLI-related diseases, disorders, or conditions.

It is another aspect of the present invention to provide methods for the treatment or prevention of several HCLI-associated diseases or disorders including, but not limited to, Myotonia congenita, retinal depolynucleotideration, male infertility, neurodepolynucleotideration, Dent's disease, X-linked nephrolithiasis syndromes, infantile malignant osteopetrosis, nephrogenic diabetes insipidus, Bartter's syndrome, renal system disorders, neurological disorders, muscular disorders and synaptic-related disorders. The methods involve administering to an individual in need of such treatment or prevention an effective amount of a modulator of the HCLI polypeptide. Preferred are HCLI antagonists. As a result of its high similarity to Parchorin and chloride channels, the HCLI molecule may be involved in ion channel-related disorders, requiring antagonism of its activity.

It is yet another aspect of this invention to provide diagnostic kits for the determination of the nucleotide sequences of human HCLI alleles. The kits comprise reagents and instructions for amplification-based assays, nucleic acid probe assays, protein nucleic acid probe assays, antibody assays or any combination thereof. Such kits are suitable for screening and for the diagnosis of disorders associated with aberrant or uncontrolled cellular proliferation or development, and with the expression of HCLI polynucleotide and encoded HCLI polypeptide in a sample, as described herein.

The above-mentioned aspects of the invention are also provided for the HCLI variant polynucleotide, HCLI.v1 (SEQ ID NO: 3), and its encoded polypeptide (SEQ ID NO: 4).

The above-mentioned aspects of the invention are also provided for the HCLI variant polynucleotide, HCLI.v2 (SEQ ID NO: 16), and its encoded polypeptide (SEQ ID NO: 17).

The invention further relates to a method for preventing, treating, or ameliorating a medical condition with the polypeptide provided as SEQ ID NO: 2, 4, or 17 in addition to, its encoding nucleic acid, or a modulator thereof, wherein the medical condition is a disorder related to altered chloride/ion homeostasis, particularly in the choroid plexus such as hyponatremia, and hypernatremia, in the lung such as cystic fibrosis, the liver such as cirrhosis, and the gall bladder such as cholecystitis.

The invention further relates to a method of diagnosing a pathological condition or a susceptibility to a pathological condition in a subject comprising the steps of (a) determining the presence or amount of expression of the polypeptide of SEQ ID NO: 2, 4, or 17 in a biological sample; (b) and diagnosing a pathological condition or a susceptibility to a pathological condition based on the presence or amount of expression of the polypeptide relative to a control, wherein said condition is a member of the group consisting of a disorder related to altered chloride/ion homeostasis, particularly in the choroid plexus such as hyponatremia, and hypernatremia, in the lung such as cystic fibrosis, the liver such as cirrhosis, and the gall bladder such as cholecystitis.

Further aspects, objects, features, and advantages of the present invention will be better understood upon a reading of the detailed description of the invention when considered in connection with the accompanying figures or drawings.

### BRIEF DESCRIPTION OF THE FIGURES

FIGS. 1A-D present the nucleic acid sequence (SEQ ID NO: 1) of the novel human intracellular chloride channel-related polynucleotide, called HCLI herein, and its encoded polypeptide sequence (SEQ ID NO: 2). Analysis of the HCLI polypeptide sequence led to the identification of a putative transmembrane domain located from about amino acid 418 to about amino acid 436 of SEQ ID NO: 2 represented by underlining, as predicted by the program TmPred. The predicted coding sequence (CDS) of HCLI comprises nucleotides 1 to 1887 of SEQ ID NO: 1.

FIGS. 2A-B present the nucleic acid sequence (SEQ ID NO: 3) of the partial novel human intracellular chloride channel-related polynucleotide variant, HCLI.v1, and its encoded polypeptide sequence (SEQ ID NO: 4). The coding sequence (CDS) of HCLI.v1 comprises nucleotides 2 to 1153.

FIGS. 3A-C present the nucleic acid sequence (SEQ ID NO: 16) of the novel human intracellular chloride channel-related polynucleotide variant, HCLI.v2, and its encoded polypeptide sequence (SEQ ID NO: 17). The coding sequence (CDS) of HCLI.v1 comprises nucleotides 1 to 2058.

FIGS. 4A-B presents the amino acid sequence alignment between HCLI (SEQ ID NO: 2), and its variants, HCLI.v1 (SEQ ID NO: 4), and HCLI.v2 (SEQ ID NO: 17). The alignment was performed using the CLUSTALW algorithm described elsewhere herein, as available within the Vector NTI AlignX program (CLUSTALW parameters: gap opening penalty: 10; gap extension penalty: 0.5; gap separation penalty range: 8; percent identity for alignment delay: 40%; and transition weighting: 0).

FIGS. 5A-5B illustrate the amino acid sequence alignment between HCLI (SEQ ID NO: 2) and its top-matching hit (Parchorin, SEQ ID NO: 5) by amino acid sequence similarity/identity using the GAP alignment program. The amino acids listed on the top lines of the alignment are amino acids of HCLI, and amino acids listed on the bottom lines of the alignment are amino acids of Parchorin. The vertical dashes between the top and bottom sequences indicate that the residues are identical, the vertical two dots between the top and bottom sequences indicate that the residues are similar, and single dots in either the top or bottom sequence lines indicate areas of non-alignment (gaps) (Example 1).

FIGS. 6A-6D illustrate a multiple sequence alignment of the amino acid sequence of HCLI (SEQ ID NO: 2), and its variants, HCLI.v1 (SEQ ID NO: 4), and HCLI.v2 (SEQ ID NO: 17), with the amino acid sequences of other Chloride Channel proteins: CICP_BOVIN (SEQ ID NO: 6), CLI4_HUMAN (SEQ ID NO: 7), CLI2_HUMAN (SEQ ID NO: 8), CLI1_HUMAN (SEQ ID NO: 9), CLI4_RAT (SEQ ID NO: 10), Parchorin (SEQ ID NO: 5), and CLI3_HUMAN (SEQ ID NO: 11). The alignment was performed using the CLUSTALW algorithm described elsewhere herein, as available within the Vector NTI AlignX program (CLUSTALW parameters: gap opening penalty: 10; gap extension penalty: 0.5; gap separation penalty range: 8; percent identity for alignment delay: 40%; and transition weighting: 0). The darkly shaded amino acids represent regions of matching identity. The lightly shaded amino acids represent regions of matching similarity. Lines between residues indicate gapped regions for the aligned polypeptides. (Example 1).

FIG. 7 presents a dendrogram summary of the amino acid alignments of FIGS. 6A-D, as polynucleotiderated by the CLUSTALW algorithm described elsewhere herein, as available within the Vector NTI AlignX program (CLUSTALW parameters: gap opening penalty: 10; gap extension penalty: 0.5; gap separation penalty range: 8; percent identity for alignment delay: 40%; and transition weighting: 0). In this dendrogram, vertical distance indicates amino acid sequence similarity, for example, HCLI and Parchorin are most similar to each other, and CLI2_HUMAN is the most similar to HCLI and Parchorin relative to the other sequences. It must be noted that similarity values are not proportional to phylopolynucleotide distances, and therefore the dendrogram of FIG. 7 is not a phylopolynucleotide tree.

FIG. 8 presents the tissue expression profile of HCLI. PCR primers (SEQ ID NO: 13 and 14) were designed from SEQ ID NO: 1 and were used to measure the steady state levels of mRNA by quantitative PCR. Transcripts corresponding to HCLI are highly expressed in heart, lung and stomach, as shown (Example 6).

FIG. 9 shows an expanded expression profile of the novel intracellular chloride ion channel, HCLI. The figure illustrates the relative expression level of HCLI amongst various mRNA tissue sources. As shown, the HCLI polypeptide was expressed predominately in the choroid-plexus (100000 to 500000 times greater than other tissues tested). Expression of HCLI was also significantly expressed in the stomach, primary and tertiary bronchus of the lung, liver, and to a lesser extent in the gallbladder. Expression data was obtained by measuring the steady state HCLI mRNA levels by quantitative PCR using the PCR primer pair provided as SEQ ID NO: 18, and 19, and Taqman probe (SEQ ID NO: 20) as described in Example 7 herein.

FIG. 10 shows an expanded expression profile of the novel intracellular chloride ion channel, HCLI, of the present invention. The figure illustrates the relative expression level of HCLI amongst various mRNA tissue sources isolated from normal and tumor tissues. As shown, the HCLI polypeptide was differentially expressed in alcoholic liver cirrhosis, and gall bladder cholecystitis tissue compared to each respective normal tissue. Expression data was obtained by measuring the steady state HCLI mRNA levels by quantitative PCR using the PCR primer pair provided as SEQ ID NO: 18, and 19, and Taqman probe (SEQ ID NO: 20) as described in Example 7 herein.

### DETAILED DESCRIPTION OF THE INVENTION

The present invention provides a novel human intracellular chloride channel-related (HCLI) polynucleotide (i.e., polynucleotide or nucleic acid sequence), (SEQ ID NO: 1) which encodes an HCLI protein (polypeptide), (SEQ ID NO: 2), preferably the full-length HCLI polypeptide. Based on percent sequence identity analysis, HCLI has been determined to be a novel intracellular chloride channel-related protein.

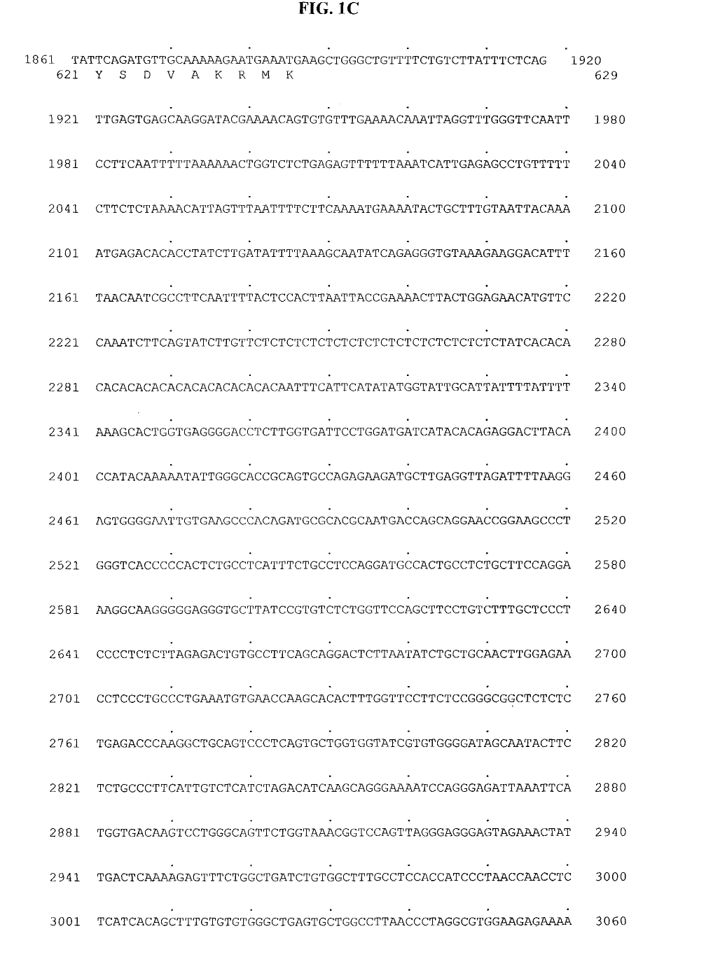

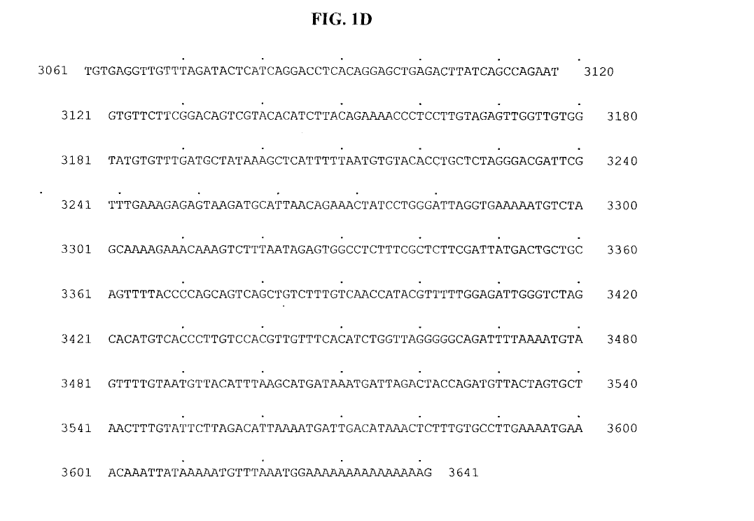

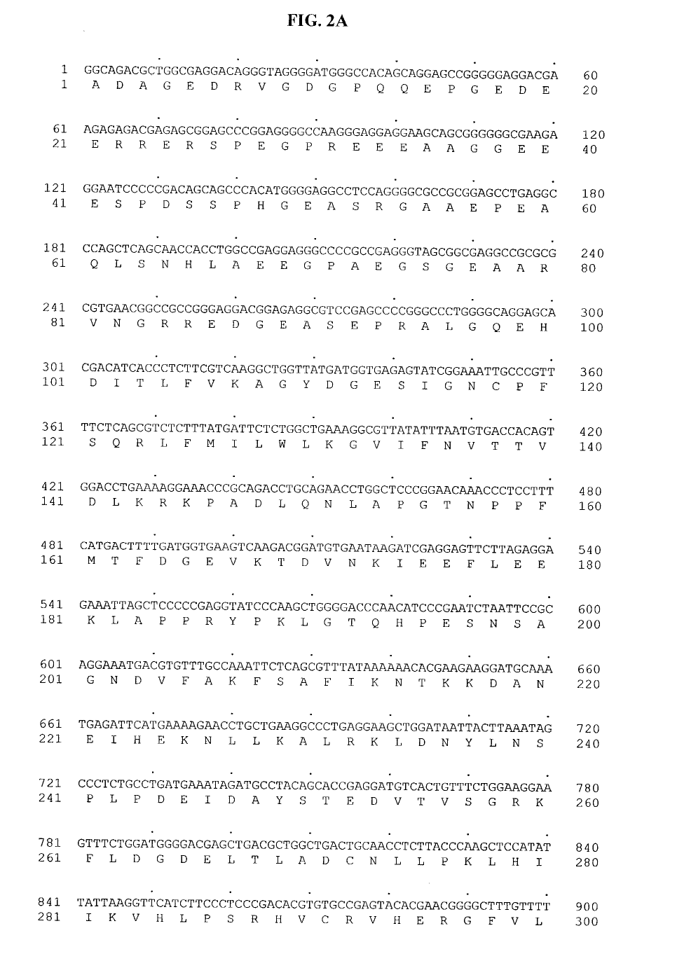

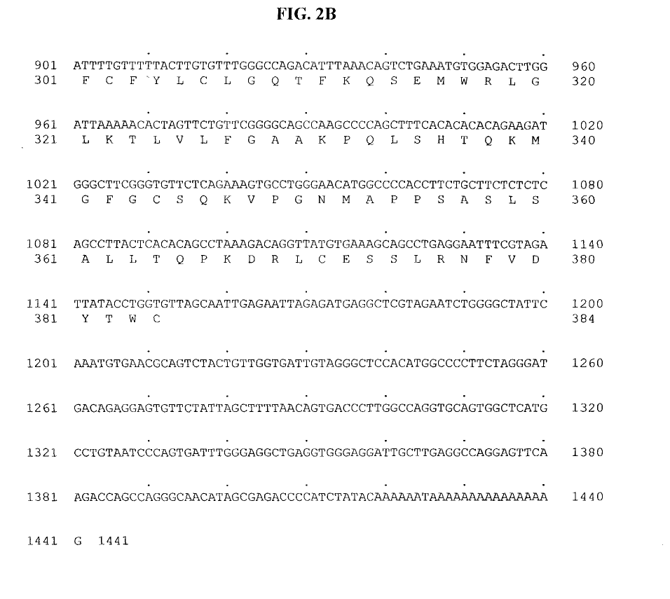

The present invention also provides an HCLI variant polynucleotide, referred to as HCLI.v1, (SEQ ID NO: 3) which encodes an HCLI variant protein (SEQ ID NO: 4). The HCLI variant protein or polypeptide contains an alternate C-terminus as compared to HCLI (see FIGS. 2A-B and Example 1).

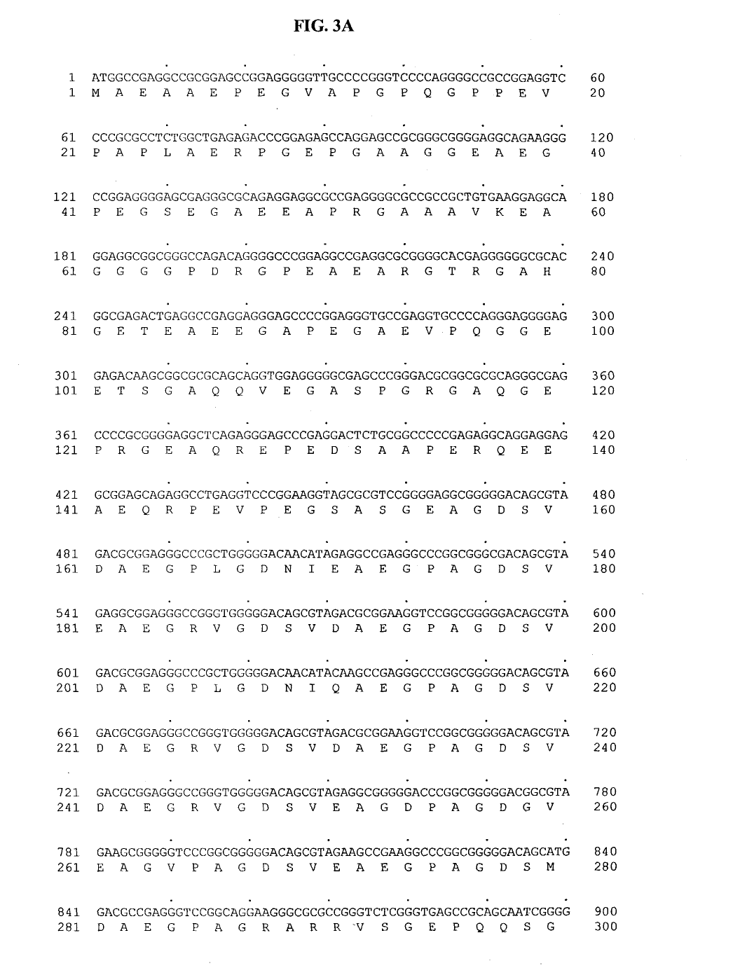

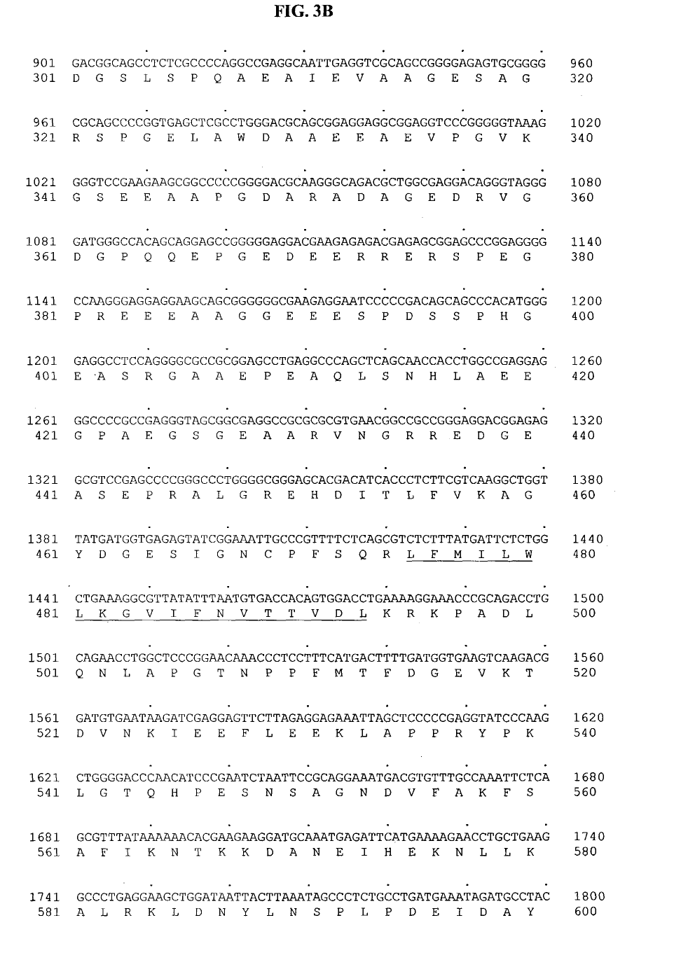

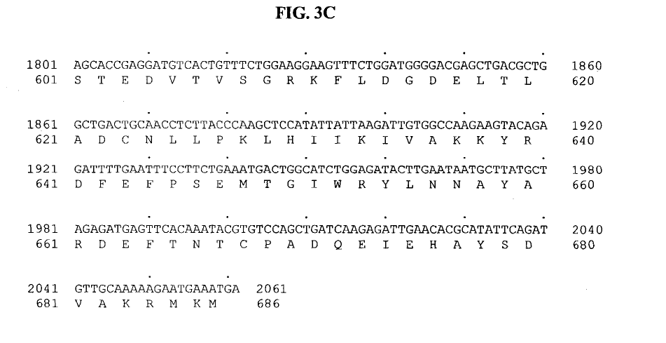

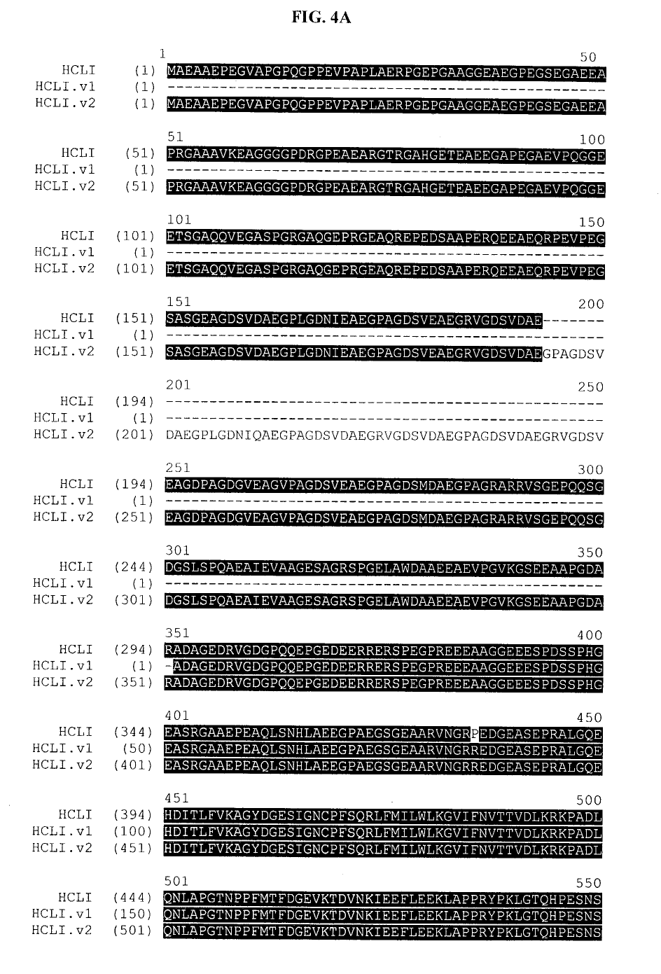

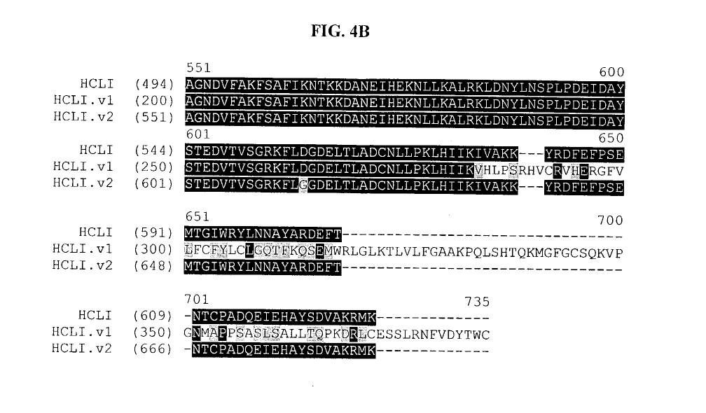

The present invention also provides an HCLI variant polynucleotide, referred to as HCLI.v2, (SEQ ID NO: 16) which encodes an HCLI variant protein (SEQ ID NO: 17). The HCLI variant protein or polypeptide contains an extra exon compared to HCLI (see FIGS. 3A-D, FIG. 4, and Example 1).

All references to “HCLI”, “SEQ ID NO: 1”, and “SEQ ID NO: 2” shall be construed to apply to HCLI, HCLIv1, HCLIv2, the polypeptide provided as SEQ ID NO: 2, the polypeptide provided as SEQ ID NO: 4, the polypeptide provided as SEQ ID NO: 17, the polynucleotide provided as SEQ ID NO: 1, the polynucleotide provided as SEQ ID NO: 3, and/or the polynucleotide provided as SEQ ID NO: 16, unless otherwise specified herein.

The invention further relates to fragments and portions of the novel HCLI nucleic acid sequence and its encoded amino acid sequence (peptides and polypeptides). Preferably, the fragments and portions of the HCLI polypeptide are functional or active. The HCLI peptides and polypeptides are useful for screening for compounds that effect the activity of HCLI. HCLI peptides and polypeptides are also useful for the polynucleotideration of specific antibodies and as bait in yeast two hybrid screens (and other protein-protein interaction screens) to identify proteins that specifically interact with HCLI.

The invention also provides methods of using the novel HCLI polynucleotide sequence and the encoded HCLI polypeptide for diagnosis, polynucleotide screening and treatment of diseases, disorders, conditions, or syndromes associated with HCLI and HCLI activity and function. The HCLI polynucleotide and polypeptide may be involved in a variety of diseases, disorders and conditions associated with HCLI activity, which include, but are not limited to, Myotonia congenita, retinal depolynucleotideration, male infertility, neurodepolynucleotideration, Dent's disease, X-linked nephrolithiasis syndromes, infantile malignant osteopetrosis, nephrogenic diabetes insipidus, Bartter's syndrome, renal system disorders, neurological disorders, muscular disorders, synaptic-related disorders and other disorders related to the dysfunction of the selective regulation of chloride transport and downstream functions such as membrane voltage, cell volume and acid secretion.

### DEFINITIONS

The following definitions are provided to more fully describe the present invention in its various aspects. The definitions are intended to be useful for guidance and elucidation, and are not intended to limit the disclosed invention or its embodiments.

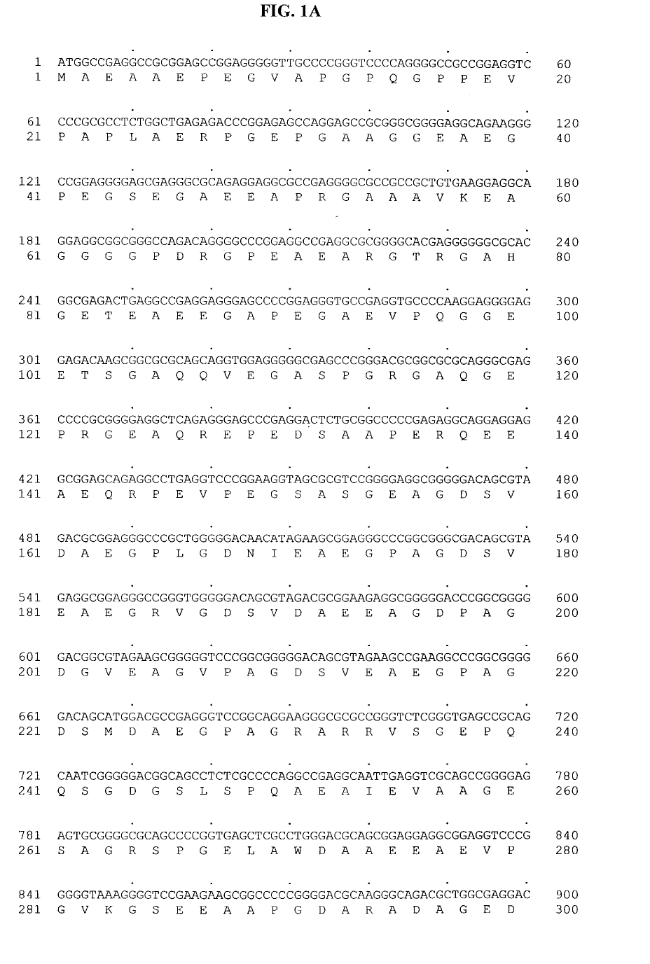

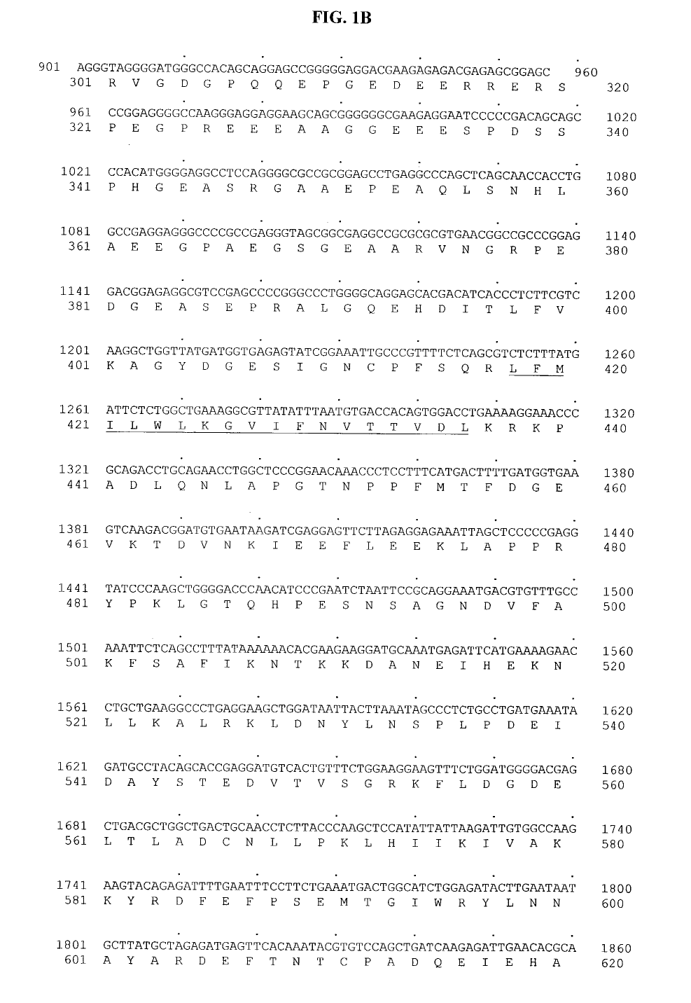

“Amino acid sequence” as used herein can refer to an oligopeptide, peptide, polypeptide, or protein sequence, and fragments or portions thereof, as well as to naturally occurring or synthetic molecules, preferably isolated polypeptides of HCLI. Amino acid sequence fragments are typically from about 4 to about 30, preferably from about 5 to about 15, more preferably from about 5 to about 15 amino acids in length and preferably retain the biological activity or function of an HCLI polypeptide. As will be appreciated by the skilled practitioner, should the amino acid fragment comprise an antigenic epitope, for example, biological function per se need not be maintained. The HCLI amino acid sequence of this invention is set forth in SEQ ID NO: 2 and as illustrated in FIGS. 1A-D. The terms HCLI polypeptide and HCLI protein are used interchangeably herein to refer to the encoded product of the HCLI nucleic acid sequence according to the present invention.

Isolated HCLI polypeptide refers to the amino acid sequence of substantially purified HCLI, which may be obtained from any species, preferably mammalian, and more preferably, human, and from a variety of sources, including natural, synthetic, semi-synthetic, or recombinant. More particularly, the HCLI polypeptide of this invention is identified in SEQ ID NO: 2. Fragments, preferably functional fragments, of the HCLI polypeptide are also embraced by the present invention.

“Similar” amino acids are those which have the same or similar physical properties and in many cases, the function is conserved with similar residues. For example, amino acids lysine and arginine are similar; while residues such as proline and cysteine do not share any physical property and are not considered to be similar.

The term “consensus” refers to a sequence that reflects the most common choice of base or amino acid at each position among a series of related DNA, RNA or protein sequences. Areas of particularly good agreement often represent conserved functional domains.

A “variant” of the HCLI polypeptide refers to an amino acid sequence that is altered by one or more amino acids. The variant may have “conservative” changes, in which a substituted amino acid has similar structural or chemical properties, e.g., replacement of leucine with isoleucine. More rarely, a variant may have “non-conservative” changes, for example, replacement of a glycine with a tryptophan. The encoded protein may also contain deletions, insertions, or substitutions of amino acid residues, which produce a silent change and result in a functionally equivalent HCLI protein. Deliberate amino acid substitutions may be made on the basis of similarity in polarity, charge, solubility, hydrophobicity, hydrophilicity, and/or the amphipathic nature of the residues, as long as the biological activity of HCLI protein is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; and phenylalanine and tyrosine. Guidance in determining which amino acid residues may be substituted, inserted, or deleted without abolishing functional biological or immunological activity may be found using computer programs well known in the art, for example, DNASTAR, Inc. software (Madison, Wis.).

The term “mimetic”, as used herein, refers to a molecule, having a structure which is developed from knowledge of the structure of the HCLI protein, or portions thereof, and as such, is able to affect some or all of the actions of the HCLI protein. A mimetic may comprise a synthetic peptide or an organic molecule.

“Nucleic acid or polynucleotide sequence”, as used herein, refers to an isolated oligonucleotide (“oligo”), nucleotide, or polynucleotide, and fragments thereof, and to DNA or RNA of genomic or synthetic origin which may be single- or double-stranded, and represent the sense or anti-sense strand, preferably of HCLI. By way of non-limiting example, fragments include nucleic acid sequences that are 20-60 nucleotides in length, or greater, and preferably include fragments that are at least 50-100 nucleotides, or which are at least 1000 nucleotides or greater in length. The HCLI nucleic acid sequence of this invention is specifically identified in SEQ ID NO: 1, and is illustrated in FIGS. 1A-D.

An “allele” or “allelic sequence” is an alternative form of the HCLI nucleic acid sequence. Alleles may result from at least one mutation in the HCLI nucleic acid sequence and may yield altered mRNAs or polypeptides whose structure or function may or may not be altered. Any given polynucleotide, whether natural or recombinant, may have none, one, or many allelic forms. Common mutational changes, which give rise to alleles, are polynucleotidely ascribed to natural deletions, additions, or substitutions of nucleotides. Each of these types of changes may occur alone, or in combination with the others, one or more times in a given sequence.

“Peptide nucleic acid” (PNA) refers to an antisense molecule or anti-polynucleotide agent which comprises an oligonucleotide (“oligo”) linked via an amide bond, similar to the peptide backbone of amino acid residues. PNAs typically comprise oligos of at least 5 nucleotides linked via amide bonds. PNAs may or may not terminate in positively charged amino acid residues to enhance binding affinities to DNA. Such amino acids include, for example, lysine and arginine, among others. These small molecules stop transcript elongation by binding to their complementary strand of nucleic acid (P. E. Nielsen et al., 1993, Anticancer Drug Des., 8:53-63). PNA may be pegylated to extend their lifespan in the cell where they preferentially bind to complementary single stranded DNA and RNA.

“Oligonucleotides” or “oligomers”, as defined herein, refer to an HCLI nucleic acid sequence comprising contiguous nucleotides of at least about 5 nucleotides to about 60 nucleotides, preferably at least about 8 to 10 nucleotides in length, more preferably at least about 12 nucleotides in length, for example, about 15 to 35 nucleotides, or about 15 to 25 nucleotides, or about 20 to 35 nucleotides, which can be typically used as probes or primers, for example, in PCR amplification assays, hybridization assays, or in microarrays. It will be understood that the term oligonucleotide is substantially equivalent to the terms primer, probe, or amplimer, as commonly defined in the art. Examples of HCLI primers of this invention are set forth SEQ ID NOS: 12-14.

The term “antisense” refers to nucleotide sequences, and compositions containing nucleic acid sequences, which are complementary to a specific DNA or RNA sequence. The term “antisense strand” is used in reference to a nucleic acid strand that is complementary to the “sense” strand. Antisense (i.e., complementary) nucleic acid molecules include PNAs and may be produced by any method, including synthesis or transcription. Once introduced into a cell, the complementary nucleotides combine with natural sequences produced by the cell to form duplexes, which block either transcription or translation. The designation “negative” is sometimes used in reference to the antisense strand, and “positive” is sometimes used in reference to the sense strand.

“Altered” nucleic acid sequences encoding an HCLI polypeptide include nucleic acid sequences containing deletions, insertions and/or substitutions of different nucleotides resulting in a polynucleotide that encodes the same or a functionally equivalent HCLI polypeptide. Altered nucleic acid sequences may further include polymorphisms of the polynucleotide encoding the HCLI polypeptide; such polymorphisms may or may not be readily detectable using a particular oligonucleotide probe.

The terms “Expressed Sequence Tag” or “EST” refers to the partial sequence of a cDNA insert which has been made by reverse transcription of mRNA extracted from a tissue, followed by insertion into a vector as known in the art (Adams, M. D., et al. Science (1991) 252:1651-1656; Adams, M. D. et al., Nature, (1992) 355:632-634; Adams, M. D., et al., Nature (1995) 377 Supp:3-174).

The term “biologically active”, i.e., functional, refers to a protein or polypeptide or fragment thereof, having structural, regulatory, or biochemical functions of a naturally occurring molecule. Likewise, “immunologically active” refers to the capability of a natural, recombinant, or synthetic HCLI polypeptide, or an oligopeptide thereof, to induce a specific immune response in appropriate animals or cells, for example, to polynucleotiderate antibodies, to bind with specific antibodies, and/or to elicit a cellular immune response.

An “agonist” refers to a molecule which, when bound to, or associated with, an HCLI polypeptide, or a functional fragment thereof, increases or prolongs the duration of the effect of the HCLI polypeptide. Agonists may include proteins, nucleic acids, carbohydrates, or any other molecules that bind to and modulate the effect of the HCLI polypeptide. Agonists typically enhance, increase, or augment,the function or activity of an HCLI molecule.

An “antagonist” refers to a molecule which, when bound to, or associated with, an HCLI polypeptide, or a functional fragment thereof, decreases or inhibits the amount or duration of the biological or immunological activity of HCLI polypeptide. Antagonists may include proteins, nucleic acids, carbohydrates, antibodies, or any other molecules that decrease or reduce the effect of an HCLI polypeptide. Antagonists typically, diminish, inhibit, or reduce the function or activity of an HCLI molecule.

As used herein the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein.

The terms “complementary” or “complementarity” refer to the natural binding of polynucleotides under permissive salt and temperature conditions by base pairing. For example, the sequence “A-G-T” binds to the complementary sequence “T-C-A”. Complementarity between two single-stranded molecules may be “partial”, in which only some of the nucleic acids bind, or it may be “complete” when total complementarity exists between single stranded molecules. The degree of complementarity between nucleic acid strands has significant effects on the efficiency and strength of hybridization between nucleic acid strands. This is of particular importance in amplification reactions, which depend upon binding between nucleic acids strands, as well as in the design and use of PNA molecules.

The term “homology” refers to a degree of complementarity. There may be partial homology or complete homology, wherein complete homology is equivalent to identity. A partially complementary sequence that at least partially inhibits an identical sequence from hybridizing to a target nucleic acid is referred to as the functional term “substantially homologous”. The inhibition of hybridization of the completely complementary sequence to the target sequence may be examined using a hybridization assay (for example, Southern or Northern blot, solution hybridization, and the like) under conditions of low stringency. A substantially homologous sequence or probe will compete for and inhibit the binding (i.e., the hybridization) of a completely homologous sequence or probe to the target sequence under conditions of low stringency. Nonetheless, conditions of low stringency do not permit non-specific binding; low stringency conditions require that the binding of two sequences to one another be a specific (i.e., selective) interaction. The absence of non-specific binding may be tested by the use of a second target sequence which lacks even a partial degree of complementarity (for example, less than about 30% identity). In the absence of non-specific binding, the probe will not hybridize to the second non-complementary target sequence.

Those having skill in the art will know how to determine percent identity between/among sequences using, for example, algorithms such as those used in the GAP computer program (S. B. Needleman and C. D. Wunsch. A polynucleotide method applicable to the search for similarities in the amino acid sequence of two proteins. J. Mol. Biol. 48(3):443-53, 1970) or based on the CLUSTALW computer program (J. D. Thompson et al., 1994, Nuc. Acids Res., 2(22):4673-4680), or FASTDB, (Brutlag et al., 1990, Comp. App. Biosci., 6:237-245), as known in the art. Although the FASTDB algorithm typically does not consider internal non-matching deletions or additions in sequences, i.e., gaps, in its calculation, this can be corrected manually to avoid an overestimation of the % identity. GAP and CLUSTALW, however, do take sequence gaps into account in their identity calculations.

The present invention is also directed to polynucleotide sequences which comprise, or alternatively consist of, a polynucleotide sequence which is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, any of the nucleotide sequences in (a), (b), (c), (d), (e), (f), (g), or (h), above. Polynucleotides encoded by these nucleic acid molecules are also encompassed by the invention. In another embodiment, the invention encompasses nucleic acid molecules which comprise, or alternatively, consist of a polynucleotide which hybridizes under stringent conditions, or alternatively, under lower stringency conditions, to a polynucleotide in (a), (b), (c), (d), (e), (f), (g), or (h), above. Polynucleotides which hybridize to the complement of these nucleic acid molecules under stringent hybridization conditions or alternatively, under lower stringency conditions, are also encompassed by the invention, as are polypeptides encoded by these polypeptides.

Another aspect of the invention provides an isolated nucleic acid molecule comprising, or alternatively, consisting of, a polynucleotide having a nucleotide sequence selected from the group consisting of: (a) a nucleotide sequence encoding a HCLI related polypeptide having an amino acid sequence as shown in the sequence listing and described herein; (b) a nucleotide sequence encoding a mature HCLI related polypeptide having the amino acid sequence as shown in the sequence listing and described herein; (c) a nucleotide sequence encoding a biologically active fragment of a HCLI related polypeptide having an amino acid sequence as shown in the sequence listing and described herein; (d) a nucleotide sequence encoding an antigenic fragment of a HCLI related polypeptide having an amino acid sequence as shown in the sequence listing and described herein; (e) a nucleotide sequence encoding a HCLI related polypeptide comprising the complete amino acid sequence encoded by a human cDNA in a cDNA plasmid contained in the ATCC Deposit and described herein; (f) a nucleotide sequence encoding a mature HCLI related polypeptide having an amino acid sequence encoded by a human cDNA in a cDNA plasmid contained in the ATCC Deposit and described herein: (g) a nucleotide sequence encoding a biologically active fragment of a HCLI related polypeptide having an amino acid sequence encoded by a human cDNA in a cDNA plasmid contained in the ATCC Deposit and described herein; (h) a nucleotide sequence encoding an antigenic fragment of a HCLI related polypeptide having an amino acid sequence encoded by a human cDNA in a cDNA plasmid contained in the ATCC deposit and described herein; (i) a nucleotide sequence complimentary to any of the nucleotide sequences in (a), (b), (c), (d), (e), (f), (g), or (h) above.

The present invention is also directed to nucleic acid molecules which comprise, or alternatively, consist of, a nucleotide sequence which is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, any of the nucleotide sequences in (a), (b), (c), (d), (e), (f), (g), or (h), above.

The present invention encompasses polypeptide sequences which comprise, or alternatively consist of, an amino acid sequence which is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, the following non-limited examples, the polypeptide sequence identified as SEQ ID NO: 2, 4, or 17, the polypeptide sequence encoded by a cDNA provided in the deposited clone, and/or polypeptide fragments of any of the polypeptides provided herein. Polynucleotides encoded by these nucleic acid molecules are also encompassed by the invention. In another embodiment, the invention encompasses nucleic acid molecules which comprise, or alternatively, consist of a polynucleotide which hybridizes under stringent conditions, or alternatively, under lower stringency conditions, to a polynucleotide in (a), (b), (c), (d), (e), (f), (g), or (h), above. Polynucleotides which hybridize to the complement of these nucleic acid molecules under stringent hybridization conditions or alternatively, under lower stringency conditions, are also encompassed by the invention, as are polypeptides encoded by these polypeptides.

The present invention is also directed to polypeptides which comprise, or alternatively consist of, an amino acid sequence which is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, the polypeptide sequence shown in SEQ ID NO: 2, 4, or 17, a polypeptide sequence encoded by the nucleotide sequence in SEQ ID NO: 1, 3, or 16, a polypeptide sequence encoded by the cDNA in cDNA plasmid: Z, and/or polypeptide fragments of any of these polypeptides (e.g., those fragments described herein). Polynucleotides which hybridize to the complement of the nucleic acid molecules encoding these polypeptides under stringent hybridization conditions or alternatively, under lower stringency conditions, are also encompasses by the present invention, as are the polypeptides encoded by these polynucleotides.

By a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence may include up to five point mutations per each 100 nucleotides of the reference nucleotide sequence encoding the polypeptide. In other words, to obtain a nucleic acid having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence referenced in herein, the ORF (open reading frame), or any fragment specified as described herein.

As a practical matter, whether any particular nucleic acid molecule or polypeptide is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both DNA sequences. An RNA sequence can be compared by converting U's to T's. However, the CLUSTALW algorithm automatically converts U's to T's when comparing RNA sequences to DNA sequences. The result of said global sequence alignment is in percent identity. Preferred parameters used in a CLUSTALW alignment of DNA sequences to calculate percent identity via pairwise alignments are: Matrix=IUB, k-tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%; Residue Specific Gaps: Off; Hydrophilic Residue Gap=Off; and Transition Weighting=0. The pairwise and multple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of 5′ or 3′ deletions, not because of internal deletions. If only the local pairwise percent identity is required, no manual correction is needed. However, a manual correction may be applied to determine the global percent identity from a global polynucleotide alignment. Percent identity calculations based upon global polynucleotide alignments are often preferred since they reflect the percent identity between the polynucleotide molecules as a whole (i.e., including any polynucleotide overhangs, not just overlapping regions), as opposed to, only local matching polynucleotides. Manual corrections for global percent identity determinations are required since the CLUSTALW program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the CLUSTALW sequence alignment. This percentage is then subtracted from the percent identity, calculated by the above CLUSTALW program using the specified parameters, to arrive at a final percent identity score. This corrected score may be used for the purposes of the present invention. Only bases outside the 5′ and 3′ bases of the subject sequence, as displayed by the CLUSTALW alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the CLUSTALW alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the CLUSTALW program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by CLUSTALW is not manually corrected. Once again, only bases 5′ and 3′ of the subject sequence which are not matched/aligned with the query sequence are manually corrected for. No other manual corrections are required for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for instance, an amino acid sequence provided in SEQ ID NO: 2, or to the amino acid sequence encoded by cDNA contained in a deposited clone, can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both amino acid sequences. The result of said global sequence alignment is in percent identity. Preferred parameters used in a CLUSTALW alignment of polypeptide sequences to calculate percent identity via pairwise alignments are: Matrix=BLOSUM, k-tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%; Residue Specific Gaps: Off; Hydrophilic Residue Gap=Off; and Transition Weighting=0. The pairwise and multple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of N- or C-terminal deletions, not because of internal deletions. If only the local pairwise percent identity is required, no manual correction is needed. However, a manual correction may be applied to determine the global percent identity from a global polypeptide alignment. Percent identity calculations based upon global polypeptide alignments are often preferred since they reflect the percent identity between the polypeptide molecules as a whole (i.e., including any polypeptide overhangs, not just overlapping regions), as opposed to, only local matching polypeptides. Manual corrections for global percent identity determinations are required since the CLUSTALW program does not account for N- and C-terminal truncations of the subject sequence when calculating percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the CLUSTALW sequence alignment. This percentage is then subtracted from the percent identity, calculated by the above CLUSTALW program using the specified parameters, to arrive at a final percent identity score. This final percent identity score is what may be used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the CLUSTALW alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the CLUSTALW program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence, which are not matched/aligned with the query. In this case the percent identity calculated by CLUSTALW is not manually corrected. Once again, only residue positions outside the N- and C-terminal ends of the subject sequence, as displayed in the CLUSTALW alignment, which are not matched/aligned with the query sequence are manually corrected for. No other manual corrections are required for the purposes of the present invention.

In addition to the above method of aligning two or more polynucleotide or polypeptide sequences to arrive at a percent identity value for the aligned sequences, it may be desirable in some circumstances to use a modified version of the CLUSTALW algorithm which takes into account known structural features of the sequences to be aligned, such as for example, the SWISS-PROT designations for each sequence. The result of such a modifed CLUSTALW algorithm may provide a more accurate value of the percent identity for two polynucleotide or polypeptide sequences. Support for such a modified version of CLUSTALW is provided within the CLUSTALW algorithm and would be readily appreciated to one of skill in the art of bioinformatics.

Also available to those having skill in this art are the BLAST and BLAST 2.0 algorithms (Altschul et al., 1977, Nuc. Acids Res., 25:3389-3402 and Altschul et al., 1990, J. Mol. Biol., 215:403-410). The BLASTN program for nucleic acid sequences uses as defaults a wordlength (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff & Henikoff, 1989, Proc. Natl. Acad. Sci., USA, 89:10915) uses alignments (B) of 50, expectation (E) of 10, M=5, N=4, and a comparison of both strands.

The term “hybridization” refers to any process by which a strand of nucleic acids binds with a complementary strand through base pairing. The term “hybridization complex” refers to a complex formed between two nucleic acid sequences by virtue of the formation of hydrogen bonds between complementary G and C bases and between complementary A and T bases. The hydrogen bonds may be further stabilized by base stacking interactions. The two complementary nucleic acid sequences hydrogen bond in an anti-parallel configuration. A hybridization complex may be formed in solution (for example, Cₒt or Rₒt analysis), or between one nucleic acid sequence present in solution and another nucleic acid sequence immobilized on a solid phase or support (for example, membranes, filters, chips, pins, or glass slides, or any other appropriate substrate to which cells or their nucleic acids have been affixed).

The terms “stringency” or “stringent conditions” refer to the conditions for hybridization as defined by nucleic acid composition, salt, and temperature. These conditions are well known in the art and may be altered to identify and/or detect identical or related polynucleotide sequences in a sample. A variety of equivalent conditions comprising either low, moderate, or high stringency depend on factors such as the length and nature of the sequence (DNA, RNA, base composition), reaction milieu (in solution or immobilized on a solid substrate), nature of the target nucleic acid (DNA, RNA, base composition), concentration of salts and the presence or absence of other reaction components (for example, formamide, dextran sulfate and/or polyethylene glycol) and reaction temperature (within a range of from about 5° C. below the melting temperature of the probe to about 20° C. to 25° C. below the melting temperature). One or more factors may be varied to polynucleotiderate conditions, either low or high stringency that is different from but equivalent to the aforementioned conditions.

As will be understood by those of skill in the art, the stringency of hybridization may be altered in order to identify or detect identical or related polynucleotide sequences. As will be further appreciated by the skilled practitioner, the melting temperature, Tₘ, can be approximated by the formulas as well known in the art, depending on a number of parameters, such as the length of the hybrid or probe in number of nucleotides, or hybridization buffer ingredients and conditions (see, for example, T. Maniatis et al., Molecular Cloning: A Laboratory Manual, Cold Spring Harbor Laboratory, Cold Spring Harbor, N.Y., 1982 and J. Sambrook et al., Molecular Cloning: A Laboratory Manual, Cold Spring Harbor Laboratory, Cold Spring Harbor, N.Y., 1989; Current Protocols in Molecular Biology, Eds. F. M. Ausubel et al., Vol. 1, “Preparation and Analysis of DNA”, John Wiley and Sons, Inc., 1994-1995, Suppls. 26, 29, 35 and 42; pp. 2.10.7-2.10.16; G. M. Wahl and S. L. Berger (1987; Methods Enzymol. 152:399-407); and A. R. Kimmel, 1987; Methods of Enzymol. 152:507-511).

As a polynucleotide guide, Tₘ decreases approximately 1° C.-1.5° C. with every 1% decrease in sequence homology. Also, in polynucleotide, the stability of a hybrid is a function of sodium ion concentration and temperature. Typically, the hybridization reaction is initially performed under conditions of low stringency, followed by washes of varying, but higher stringency. Reference to hybridization stringency, for example, high, moderate, or low stringency, typically relates to such washing conditions. It is to be understood that the low, moderate and high stringency hybridization or washing conditions can be varied using a variety of ingredients, buffers and temperatures well known to and practiced by the skilled artisan.

A “composition”, as defined herein, refers broadly to any composition containing an HCLI polynucleotide, polypeptide, derivative, or mimetic thereof, or antibodies thereto. The composition may comprise a dry formulation or an aqueous solution. Compositions comprising the HCLI polynucleotide sequence (SEQ ID NO: 1) encoding HCLI polypeptide (SEQ ID NO: 2), or fragments thereof, may be employed as hybridization probes. The probes may be stored in a freeze-dried form and may be in association with a stabilizing agent such as a carbohydrate. In hybridizations, the probe may be employed in an aqueous solution containing salts (for example, NaCl), detergents or surfactants (for example, SDS) and other components (for example, Denhardt's solution, dry milk, salmon sperm DNA, and the like).

The term “substantially purified” refers to nucleic acid sequences or amino acid sequences that are removed from their natural environment, isolated or separated, and are at least 60% free, preferably 75% to 85% free, and most preferably 90% to 95%, or greater, free from other components with which they are naturally associated.

The term “sample”, or “biological sample”, is meant to be interpreted in its broadest sense. A non-limiting example of a biological sample suspected of containing an HCLI nucleic acid encoding HCLI protein, or fragments thereof, or an HCLI protein itself, may comprise, but is not limited to, a body fluid, an extract from cells or tissue, chromosomes isolated from a cell (for example, a spread of metaphase chromosomes), organelle, or membrane isolated from a cell, a cell, nucleic acid such as genomic HCLI DNA (in solution or bound to a solid support such as, for example, for Southern analysis), HCLI RNA (in solution or bound to a solid support such as for Northern analysis), HCLI cDNA (in solution or bound to a solid support), a tissue, a tissue print, and the like.

“Transformation” or transfection refers to a process by which exogenous DNA, preferably HCLI DNA, enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and partial bombardment. Such “transformed” cells include stably transformed cells in which the inserted DNA is capable of replication either as an autonomously replicating plasmid or as part of the host chromosome. Transformed cells also include those cells, which transiently express the inserted DNA or RNA for limited periods of time.

The term “correlates with expression of a polynucleotide” indicates that the detection of the presence of ribonucleic acid that is similar to the nucleic acid sequence of HCLI by Northern analysis is indicative of the presence of mRNA encoding HCLI polypeptide (SEQ ID NO: 2) in a sample and thereby correlates with expression of the transcript from the polynucleotide encoding the protein.

An alteration in the polynucleotide of SEQ ID NO: 1 comprises any alteration in the sequence of the polynucleotide encoding HCLI polypeptide, including deletions, insertions, and point mutations that may be detected using hybridization assays. Included within this definition is the detection of alterations to the genomic DNA sequence which encodes the HCLI polypeptide (e.g., by alterations in the pattern of restriction fragment length polymorphisms capable of hybridizing to nucleic acid sequences of SEQ ID NO: 1), the inability of a selected fragment of SEQ ID NO: 1 to hybridize to a sample of genomic DNA (e.g., using allele-specific oligonucleotide probes), and improper or unexpected hybridization, such as hybridization to a locus other than the normal chromosomal locus for the polynucleotide sequence encoding the HCLI polypeptide (e.g., using fluorescent in situ hybridization (FISH) to metaphase chromosome spreads).

The term “antibody” refers to intact molecules as well as fragments thereof, such as Fab, F(ab′)₂, Fv, which are capable of binding an epitopic or antigenic determinant. Antibodies that bind to an HCLI polypeptide can be prepared using intact polypeptides or fragments containing small peptides of interest or prepared recombinantly for use as the immunizing antigen. The polypeptide or oligopeptide used to immunize an animal can be derived from the transition of RNA or synthesized chemically, and can be conjugated to a carrier protein, if desired. Commonly used carriers that are chemically coupled to peptides include, but are not limited to, bovine serum albumin (BSA), keyhole limpet hemocyanin (KLH), and thyroglobulin. The coupled peptide is then used to immunize the animal (for example, a mouse, a rat, or a rabbit).

The term “humanized” antibody refers to antibody molecules in which amino acids have been replaced in the non-antigen binding regions (i.e., framework regions) of the immunoglobulin in order to more closely resemble a human antibody, while still retaining the original binding capability, for example, as described in U.S. Pat. No. 5,585,089 to C. L. Queen et al. In the present instance, humanized antibodies are preferably anti-HCLI specific antibodies.

The term “antigenic determinant” refers to that portion of a molecule that makes contact with a particular antibody (i.e., an epitope). When a protein or fragment of a protein, preferably an HCLI protein, is used to immunize a host animal, numerous regions of the protein may induce the production of antibodies which bind specifically to a given region or three-dimensional structure on the protein; these regions or structures are referred to an antigenic determinants. An antigenic determinant may compete with the intact antigen (i.e., the immunogen used to elicit the immune response) for binding to an antibody.

The terms “specific binding” or “specifically binding” refer to the interaction between a protein or peptide, preferably an HCLI protein, and a binding molecule, such as an agonist, an antagonist, or an antibody. The interaction is dependent upon the presence of a particular structure (i.e., an antigenic determinant or epitope) of the protein that is recognized by the binding molecule.

### DESCRIPTION OF THE INVENTION

The present invention provides a novel HCLI polynucleotide (SEQ ID NO: 1) and its encoded HCLI polypeptide (SEQ ID NO: 2). The HCLI according to this invention is preferably a full-length molecule. The HCLI according to the invention is a member of the ion channel superfamily and the chloride ion channel family. More specifically, HCLI is an intracellular chloride ion channel-related protein.

The HCLI polynucleotide and/or polypeptide of this invention are useful for diagnosing diseases related to over- or under-expression of the HCLI protein. For example, such HCLI-associated diseases can be assessed by identifying mutations in the HCLI polynucleotide using HCLI probes or primers, or by determining HCLI protein or mRNA expression levels. An HCLI polypeptide is also useful for screening compounds which affect activity of the protein. The invention further encompasses the polynucleotide encoding the HCLI polypeptide and the use of the HCLI polynucleotide or polypeptide, or compositions thereof, in the screening, diagnosis, treatment, or prevention of disorders associated with aberrant or uncontrolled regulation of membrane potential and chloride anion transport. HCLI probes or primers can be used, for example, to screen for diseases associated with HCLI expression.

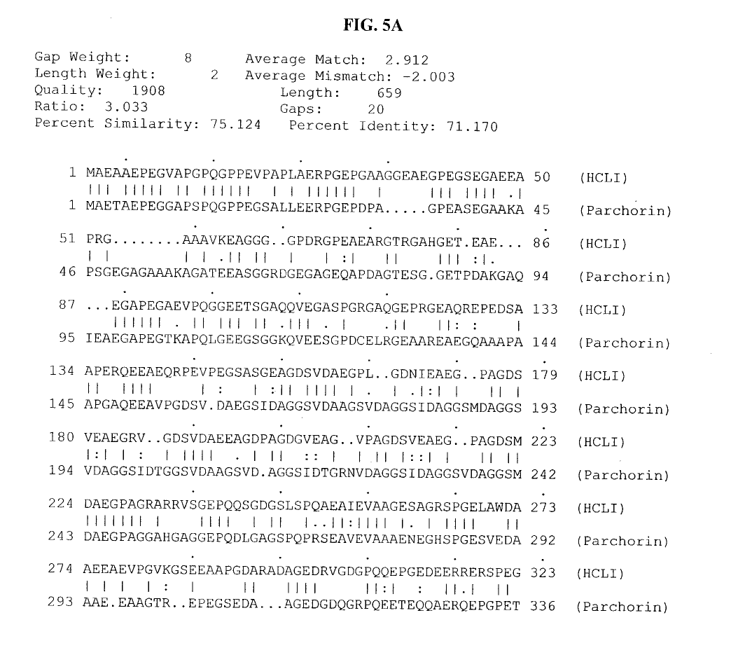

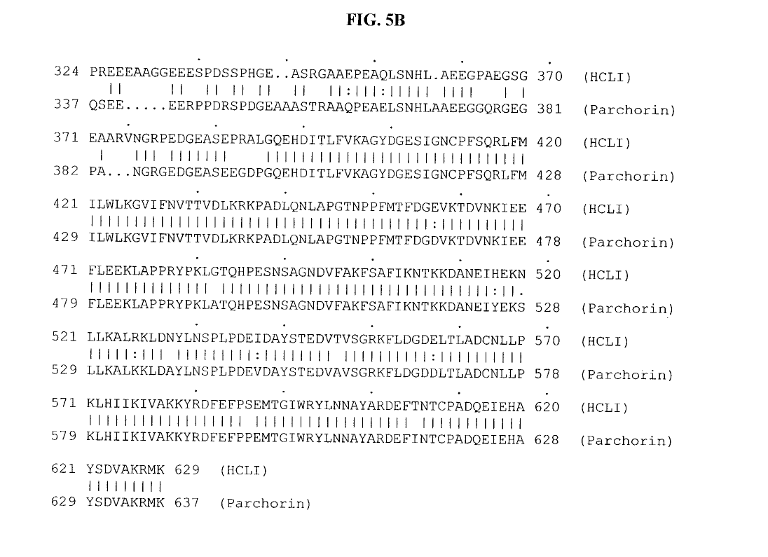

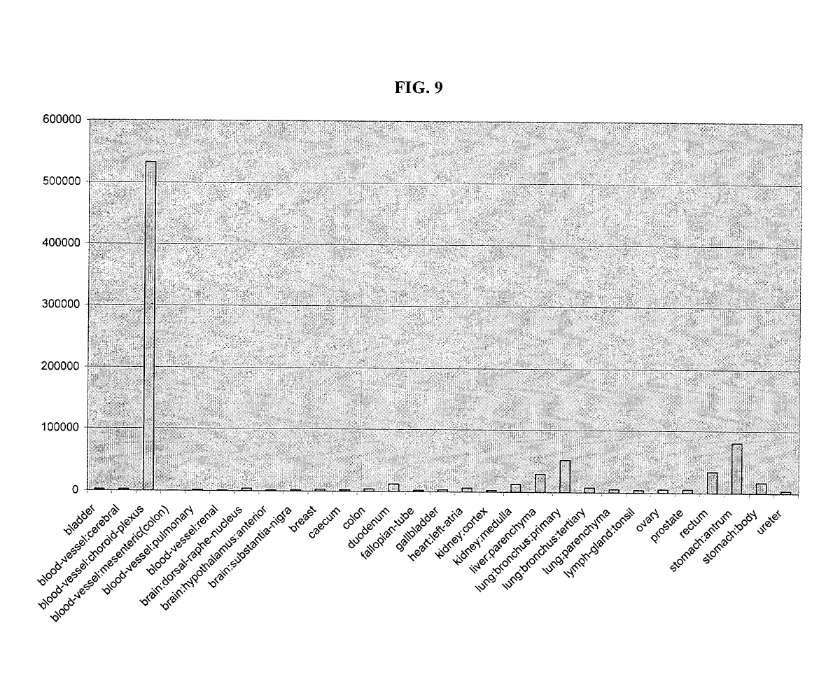

One embodiment of the present invention encompasses a novel HCLI polypeptide comprising the amino acid sequence of SEQ ID NO: 2 as shown in FIGS. 1A-D. More specifically, the HCLI polypeptide of SEQ ID NO: 2 is 629 amino acids in length with a predicted molecular weight of 65.6 kilodaltons and has 71% local amino acid sequence identity and 75% local amino acid sequence similarity, (FIGS. 5A-5B), with the rabbit intracellular chloride channel related protein, Parchorin (SEQ ID NO: 5, FIG. 9). HCLI also shows significant homology to other chloride ion channels (FIGS. 7A-7C). The Parchorin protein mainly localizes to the cytosol, but translocates to the plasma membrane to function in the regulation of chloride transport (Nishizawa, T. et al., J. Biol. Chem., 275:11164-11173, (2000)). Like the Parchorin protein, the amino terminus of HCLI is acidic. The predicted isoelectric point of HCLI is 4.11, suggesting that even though there is a putative transmembrane region in HCLI, the protein is most likely intracellular in distribution. HCLI may play a role in motility by setting the membrane potential, and therefore determining excitability in smooth muscle cells, interstitial cells of Cajal, and enteric neurons located within the GI tract. As HCLI is highly expressed in stomach, lung and heart, conditions associated with the dysfunction of chloride transport and excitability in these tissues are particularly relevant.

Another embodiment of the present invention encompasses a novel HCLI variant polypeptide. HCLI.v1, comprising the amino acid sequence of SEQ ID NO: 4 as shown in FIGS. 2A-B, and encoded by the nucleotide sequence of SEQ ID NO: 3. More specifically, the HCLI variant polypeptide of SEQ ID NO: 4 is 384 amino acids in length and has 80% local amino acid sequence identity with HCLI (see FIG. 4).

Another embodiment of the present invention encompasses a novel HCLI variant polypeptide, HCLI.v2, comprising the amino acid sequence of SEQ ID NO: 17 as shown in FIGS. 3A-D, and encoded by the nucleotide sequence of SEQ ID NO: 16. More specifically, the HCLI variant polypeptide of SEQ ID NO: 17 is 686 amino acids in length and has 91% local amino acid sequence identity with HCLI (see FIG. 4).

Other variants of HCLI polypeptide are also encompassed by the present invention, such as an HCLI variant polypeptide comprising the amino acid sequence of SEQ ID NO: 4 as shown in FIGS. 2A-B and the HCLI variant nucleic acid (SEQ ID NO: 3; FIG. 3) which encodes SEQ ID NO: 4, in addition to the HCLI variant polypeptide comprising the amino acid sequence of SEQ ID NO: 17 as shown in FIGS. 3A-D and the HCLI variant nucleic acid (SEQ ID NO: 16; FIGS. 3A-D) which encodes SEQ ID NO: 17. Preferably, an HCLI variant has at least 75 to 80%, more preferably at least 85 to 90%, and even more preferably at least 90% amino acid sequence identity to the HCLI amino acid sequence disclosed herein, and more preferably, retains at least one biological, immunological, or other functional characteristic or activity of the non-variant HCLI polypeptide. Most preferred are HCLI variants or substantially purified fragments thereof having at least 95% amino acid sequence identity to that of SEQ ID NO:2. Variants of the HCLI polypeptide, or substantially purified fragments of the polypeptide, can also include amino acid sequences that differ from the SEQ ID NO: 2, SEQ ID NO: 4, or SEQ ID NO: 17 amino acid sequence only by conservative substitutions. The invention also encompasses polypeptide homologues of the amino acid sequence as set forth in SEQ ID NO: 2, SEQ ID NO: 4, or SEQ ID NO: 17.

In another embodiment, the present invention encompasses polynucleotides which encode HCLI polypeptides. Accordingly, any nucleic acid sequence that encodes the amino acid sequence of an HCLI polypeptide of the invention can be used to produce recombinant molecules that express an HCLI protein. More particularly, the invention encompasses the HCLI polynucleotide having the nucleic acid sequence of SEQ ID NO: 1. The present invention also provides a clone containing HCLI cDNA, deposited at the American Type Culture Collection (ATCC), 10801 University Boulevard, Manassas, Va. 20110-2209 on Nov. 14, 2002, and under ATCC Accession No(s). PTA-4803 according to the terms of the Budapest Treaty.

As will be appreciated by the skilled practitioner in the art, the depolynucleotideracy of the polynucleotide code results in many nucleotide sequences that can encode the described HCLI polypeptide. Some of the sequences bear minimal or no homology to the nucleotide sequences of any known and naturally occurring polynucleotide. Accordingly, the present invention contemplates each and every possible variation of nucleotide sequence that could be made by selecting combinations based on possible codon choices. These combinations are made in accordance with the standard triplet polynucleotide code as applied to the nucleotide sequence of naturally occurring HCLI, and all such variations are to be considered as being specifically disclosed and able to be understood by the skilled practitioner.

In preferred embodiments, the following N-terminal HCLI deletion polypeptides are encompassed by the present invention: M1-K629, A2-K629, E3-K629, A4-K629, A5-K629, E6-K629, P7-K629, E8-K629, G9-K629, V10-K629, A11-K629, P12-K629, G13-K629, P14-K629, Q15-K629, G16-K629, P17-K629, P18-K629, E19-K629, V20-K629, P21-K629, A22-K629, P23-K629, L24-K629, A25-K629, E26-K629, R27-K629, P28-K629, G29-K629, E30-K629, P31-K629, G32-K629, A33-K629, A34-K629, G35-K629, G36-K629, E37-K629, A38-K629, E39-K629, G40-K629, P41-K629, E42-K629, G43-K629, S44-K629, E45-K629, G46-K629, A47-K629, E48-K629, E49-K629, A50-K629, P51-K629, R52-K629, G53-K629, A54-K629, A55-K629, A56-K629, V57-K629, K58-K629, E59-K629, A60-K629, G61-K629, G62-K629, G63-K629, G64-K629, P65-K629, D66-K629, R67-K629, G68-K629, P69-K629, E70-K629, A71-K629, E72-K629, A73-K629, R74-K629, G75-K629, T76-K629, R77-K629, G78-K629, A79-K629, H80-K629, G81-K629, E82-K629, T83-K629, E84-K629, A85-K629, E86-K629, E87-K629, G88-K629, A89-K629, P90-K629, E91-K629, G92-K629, A93-K629, E94-K629, V95-K629, P96-K629, Q97-K629, G98-K629, G99-K629, E100-K629, E101-K629, T102-K629, S103-K629, G104-K629, A105-K629, Q106-K629, Q107-K629, V108-K629, E109-K629, G110-K629, A111-K629, S112-K629, P113-K629, G 114-K629, R115-K629, G116-K629, A117-K629, Q118-K629, G119-K629, E120-K629, P121-K629, R122-K629, G123-K629, E124-K629, A125-K629, Q126-K629, R127-K629, E128-K629, P129-K629, E130-K629, D131-K629, S132-K629, A133-K629, A134-K629, P135-K629, E136-K629, R137-K629, Q138-K629, E139-K629, E140-K629, A141-K629, E142-K629, Q143-K629, R144-K629, P145-K629, E146-K629, V147-K629, P148-K629, E149-K629, G150-K629, S151-K629, A152-K629, S153-K629, G154-K629, E155-K629, A156-K629, G157-K629, D158-K629, S159-K629, V160-K629, D161-K629, A162-K629, E163-K629, G164-K629, P165-K629, L166-K629, G167-K629, D168-K629, N169-K629, I170-K629, E171-K629, A172-K629, E173-K629, G174-K629, P175-K629, A176-K629, G177-K629, D178-K629, S179-K629, V180-K629, E181-K629, A182-K629, E183-K629, G184-K629, R185-K629, V186-K629, G187-K629, D188-K629, S189-K629, V190-K629, D191-K629, A192-K629, E193-K629, E194-K629, A195-K629, G196-K629, D197-K629, P198-K629, A199-K629, G200-K629, D201-K629, G202-K629, V203-K629, E204-K629, A205-K629, G206-K629, V207-K629, P208-K629, A209-K629, G210-K629, D211-K629, S212-K629, V213-K629, E214-K629, A215-K629, E216-K629, G217-K629, P218-K629, A219-K629, G220-K629, D221-K629, S222-K629, M223-K629, D224-K629, A225-K629, E226-K629, G227-K629, P228-K629, A229-K629, G230-K629, R231-K629, A232-K629, R233-K629, R234-K629, V235-K629, S236-K629, G237-K629, E238-K629, P239-K629, Q240-K629, Q241-K629, S242-K629, G243-K629, D244-K629, G245-K629, S246-K629, L247-K629, S248-K629, P249-K629, Q250-K629, A251-K629, E252-K629, A253-K629, I254-K629, E255-K629, V256-K629, A257-K629, A258-K629, G259-K629, E260-K629, S261-K629, A262-K629, G263-K629, R264-K629, S265-K629, P266-K629, G267-K629, E268-K629, L269-K629, A270-K629, W271-K629, D272-K629, A273-K629, A274-K629, E275-K629, E276-K629, A277-K629, E278-K629, V279-K629, P280-K629, G281-K629, V282-K629, K283-K629, G284-K629, S285-K629, E286-K629, E287-K629, A288-K629, A289-K629, P290-K629, G291-K629, D292-K629, A293-K629, R294-K629, A295-K629, D296-K629, A297-K629, G298-K629, E299-K629, D300-K629, R301-K629, V302-K629, G303-K629, D304-K629, G305-K629, P306-K629, Q307-K629, Q308-K629, E309-K629, P310-K629, G311-K629, E312-K629, D313-K629, E314-K629, E315-K629, R316-K629, R317-K629, E318-K629, R319-K629, S320-K629, P321-K629, E322-K629, G323-K629, P324-K629, R325-K629, E326-K629, E327-K629, E328-K629, A329-K629, A330-K629, G331-K629, G332-K629, E333-K629, E334-K629, E335-K629, S336-K629, P337-K629, D338-K629, S339-K629, S340-K629, P341-K629, H342-K629, G343-K629, E344-K629, A345-K629, S346-K629, R347-K629, G348-K629, A349-K629, A350-K629, E351-K629, P352-K629, E353-K629, A354-K629, Q355-K629, L356-K629, S357-K629, N358-K629, H359-K629, L360-K629, A361-K629, E362-K629, E363-K629, G364-K629, P365-K629, A366-K629, E367-K629, G368-K629, S369-K629, G370-K629, E371-K629, A372-K629, A373-K629, R374-K629, V375-K629, N376-K629, G377-K629, R378-K629, P379-K629, E380-K629, D381-K629, G382-K629, E383-K629, A384-K629, S385-K629, E386-K629, P387-K629, R388-K629, A389-K629, L390-K629, G391-K629, Q392-K629, E393-K629, H394-K629, D395-K629, I396-K629, T397-K629, L398-K629, F399-K629, V400-K629, K401-K629, A402-K629, G403-K629, Y404-K629, D405-K629, G406-K629, E407-K629, S408-K629, I409-K629, G410-K629, N411-K629, C412-K629, P413-K629, F414-K629, S415-K629, Q416-K629, R417-K629, L418-K629, F419-K629, M420-K629, I421-K629, L422-K629, W423-K629, L424-K629, K425-K629, G426-K629, V427-K629, I428-K629, F429-K629, N430-K629, V431-K629, T432-K629, T433-K629, V434-K629, D435-K629, L436-K629, K437-K629, R438-K629, K439-K629, P440-K629, A441-K629, D442-K629, L443-K629, Q444-K629, N445-K629, L446-K629, A447-K629, P448-K629, G449-K629, T450-K629, N451-K629, P452-K629, P453-K629, F454-K629, M455-K629, T456-K629, F457-K629, D458-K629, G459-K629, E460-K629, V461-K629, K462-K629, T463-K629, D464-K629, V465-K629, N466-K629, K467-K629, I468-K629, E469-K629, E470-K629, F471-K629, L472-K629, E473-K629, E474-K629, K475-K629, L476-K629, A477-K629, P478-K629, P479-K629, R480-K629, Y481-K629, P482-K629, K483-K629, L484-K629, G448-K629, T486-K629, Q487-K629, H488-K629, P489-K629, E490-K629, S491-K629, N492-K629, S493-K629, A494-K629, G495-K629, N496-K629, D497-K629, V498-K629, F499-K629, A500-K629, K501-K629, F502-K629, S503-K629, A504-K629, F505-K629, I506-K629, K507-K629, N508-K629, T509-K629, K510-K629, K511-K629, D512-K629, A513-K629, N514-K629, E515-K629, I516-K629, H517-K629, E518-K629, K519-K629, N520-K629, L521-K629, L522-K629, K523-K629, A524-K629, L525-K629, R526-K629, K527-K629, L528-K629, D529-K629, N530-K629, Y531-K629, L532-K629, N533-K629, S534-K629, P535-K629, L536-K629, P537-K629, D538-K629, E539-K629, I540-K629, D541-K629, A542-K629, Y543-K629, S544-K629, T545-K629, E546-K629, D547-K629, V548-K629, T549-K629, V550-K629, S551-K629, G552-K629, R553-K629, K554-K629, F555-K629, L556-K629, D557-K629, G558-K629, D559-K629, E560-K629, L561-K629, T562-K629, L563-K629, A564-K629, D565-K629, C566-K629, N567-K629, L568-K629, L569-K629, P570-K629, K571-K629, L572-K629, H573-K629, I574-K629, I575-K629, K576-K629, I577-K629, V578-K629, A579-K629, K580-K629, K581-K629, Y582-K629, R583-K629, D584-K629, F585-K629, E586-K629, F587-K629, P588-K629, S589-K629, E590-K629, M591-K629, T592-K629, G593-K629, I594-K629, W595-K629, R596-K629, Y597-K629, L598-K629, N599-K629, N600-K629, A601-K629, Y602-K629, A603-K629, R604-K629, D605-K629, E606-K629, F607-K629, T608-K629, N609-K629, T610-K629, C611-K629, P612-K629, A613-K629, D614-K629, Q615-K629, E616-K629, I617-K629, E618-K629, H619-K629, A620-K629, Y621-K629, S622-K629, and/or D623-K629 of SEQ ID NO: 2. Polynucleotide sequences encoding these polypeptides are also provided. The present invention also encompasses the use of these N-terminal HCLI deletion polypeptides as immunogenic and/or antigenic epitopes as described elsewhere herein.

In preferred embodiments, the following C-terminal HCLI deletion polypeptides are encompassed by the present invention: M1-K629, M1-M628, M1-R627, M1-K626, M1-A625, M1-V624, M1-D623, M1-S622, M1-Y621, M1-A620, M1-H619, M1-E618, M1-I617, M1-E616, M1-Q615, M1-D614, M1-A613, M1-P612, M1-C611, M1-T610, M1-N609, M1-T608, M1-F607, M1-E606, M1-D605, M1-R604, M1-A603, M1-Y602, M1-A601, M1-N600, M1-N599, M1-L598, M1-Y597, M1-R596, M1-W595, M1-I594, M1-G593, M1-T592, M1-M591, M1-E590, M1-S589, M1-P588, M1-F587, M1-E586, M1-F585, M1-D584, M1-R583, M1-Y582, M1-K581, M1-K580, M1-A579, M1-V578, M1-I577, M1-K576, M1-I575, M1-I574, M1-H573, M1-L572, M1-K571, M1-P570, M1-L569, M1-L568, M1-N567, M1-C566, M1-D565, M1-A564, M1-L563, M1-T562, M1-L561, M1-E560, M1-D559, M1-G558, M1-D557, M1-L556, M1-F555, M1-K554, M1-R553, M1-G522, M1-S551, M1-V550, M1-T549, M1-V548, M1-D547, M1-E546, M1-T545, M1-S544, M1-Y543, M1-A542, M1-D541, M1-I540, M1-E539, M1-D538, M1-P537, M1-L536, M1-P535, M1-S534, M1-N533, M1-L532, M1-Y531, M1-N530, M1-D529, M1-L528, M1-K527, M1-R526, M1-L525, M1-A524, M1-K523, M1-L522, M1-L521, M1-N520, M1-K519, M1-E518, M1-H517, M1-I516, M1-E515, M1-N514, M1-A513, M1-D512, M1-K511, M1-K510, M1-T509, M1-N508, M1-K507, M1-I506, M1-F505, M1-A504, M1-S503, M1-F502, M1-K501, M1-A500, M1-F499, M1-V498, M1-D497, M1-N496, M1-G495, M1-A494, M1-S493, M1-N492, M1-S491, M1-E490, M1-P489, M1-H488, M1-Q487, M1-T486, M1-G485, M1-L484, M1-K483, M1-P482, M1-Y481, M1-R480, M1-P479, M1-P478, M1-A477, M1-L476, M1-K475, M1-E474, M1-E473, M1-L472, M1-F471, M1-E470, M1-E469, M1-I468, M1-K467, M1-N466, M1-V465, M1-D464, M1-T463, M1-K462, M1-V461, M1-E460, M1-G459, M1-D458, M1-F457, M1-T456, M1-M455, M1-F454, M1-P453, M1-P452, M1-N451, M1-T450, M1-G449, M1-P448, M1-A447, M1-L446, M1-N445, M1-Q444, M1-L443, M1-D442, M1-A441, M1-P440, M1-K439, M1-R438, M1-K437, M1-L436, M1-D435, M1-V434, M1-T433, M1-T432, M1-V431, M1-N430, M1-F429, M1-I428, M1-V427, M1-G426, M1-K425, M1-L424, M1-W423, M1-L422, M1-I421, M1-M420, M1-F419, M1-L418, M1-R417, M1-Q416, M1-S415, M1-F414, M1-P413, M1-C412, M1-N411, M1-G410, M1-I409, M1-S408, M1-E407, M1-G406, M1-D405, M1-Y404, M1-G403, M1-A402, M1-K401, M1-V400, M1-F399, M1-L398, M1-T397, M1-I396, M1-D395, M1-H394, M1-E393, M1-Q392, M1-G391, M1-L390, M1-A389, M1-R388, M1-P387, M1-E386, M1-S385, M1-A384, M1-E383, M1-G382, M1-D381, M1-E380, M1-P379, M1-R378, M1-G377, M1-N376, M1-V375, M1-R374, M1-A373, M1-A372, M1-E371, M1-G370, M1-S369, M1-G368, M1-E367, M1-A366, M1-P365, M1-G364, M1-E363, M1-E362, M1-A361, M1-L360, M1-H359, M1-N358, M1-S357, M1-L356, M1-Q355, M1-A354, M1-E353, M1-P352, M1-E351, M1-A350, M1-A349, M1-G348, M1-R347, M1-S346, M1-A345, M1-E344, M1-G343, M1-H342, M1-P341, M1-S340, M1-S339, M1-D338, M1-P337, M1-S336, M1-E335, M1-E334, M1-E333, M1-G332, M1-G331, M1-A330, M1-A329, M1-E328, M1-E327, M1-E326, M1-R325, M1-P324, M1-G323, M1-E322, M1-P321, M1-S320, M1-R319, M1-E318, M1-R317, M1-R316, M1-E315, M1-E314, M1-D313, M1-E312, M1-G311, M1-P310, M1-E309, M1-Q308, M1-Q307, M1-P306, M1-G305, M1-D304, M1-G303, M1-V302, M1-R301, M1-D300, M1-E299, M1-G298, M1-A297, M1-D296, M1-A295, M1-R294, M1-A293, M1-D292, M1-G291, M1-P290, M1-A289, M1-A288, M1-E287, M1-E286, M1-S285, M1-G284, M1-K283, M1-V282, M1-G281, M1-P280, M1-V279, M1-E278, M1-A277, M1-E276, M1-E275, M1-A274, M1-A273, M1-D272, M1-W271, M1-A270, M1-L269, M1-E268, M1-G267, M1-P266, M1-S265, M1-R264, M1-G263, M1-A262, M1-S261, M1-E260, M1-G259, M1-A258, M1-A257, M1-V256, M1-E255, M1-I254, M1-A253, M1-E252, M1-A251, M1-Q250, M1-P249, M1-S248, M1-L247, M1-S246, M1-G245, M1-D244, M1-G243, M1-S242, M1-Q241, M1-Q240, M1-P239, M1-E238, M1-G237, M1-S236, M1-V235, M1-R234, M1-R233, M1-A232, M1-R231, M1-G230, M1-A229, M1-P228, M1-G227, M1-E226, M1-A225, M1-D224, M1-M223, M1-S222, M1-D221, M1-G220, M1-A219, M1-P218, M1-G217, M1-E216, M1-A215, M1-E214, M1-V213, M1-S212, M1-D211, M1-G210, M1-A209, M1-P208, M1-V207, M1-G206, M1-A205, M1-E204, M1-V203, M1-G202, M1-D201, M1-G200, M1-A199, M1-P198, M1-D197, M1-G196, M1-A195, M1-E194, M1-E193, M1-A192, M1-D191, M1-V190, M1-S189, M1-D188, M1-G187, M1-V186, M1-R185, M1-G184, M1-E183, M1-A182, M1-E181, M1-V180, M1-S179, M1-D178, M1-G177, M1-A176, M1-P175, M1-G174, M1-E173, M1-A172, M1-E171, M1-I170, M1-N169, M1-D168, M1-G167, M1-L166, M1-P165, M1-G164, M1-E163, M1-A162, M1-D161, M1-V160, M1-S159, M1-D158, M1-G157, M1-A156, M1-E155, M1-G154, M1-S153, M1-A152, M1-S151, M1-G150, M1-E149, M1-P148, M1-V147, M1-E146, M1-P145, M1-R144, M1-Q143, M1-E142, M1-A141, M1-E140, M1-E139, M1-Q138, M1-R137, M1-E136, M1-P135, M1-A134, M1-A133, M1-S132, M1-D131, M1-E130, M1-P129, M1-E128, M1-R127, M1-Q126, M1-A125, M1-E124, M1-G123, M1-R122, M1-P121, M1-E120, M1-G119, M1-Q118, M1-A117, M1-G116, M1-R115, M1-G114, M1-P113, M1-S112, M1-A111, M1-G110, M1-E109, M1-V108, M1-Q107, M1-Q106, M1-A105, M1-G104, M1-S103, M1-T102, M1-E101, M1-E100, M1-G99, M1-G98, M1-Q97, M1-P96, M1-V95, M1-E94, M1-A93, M1-G92, M1-E91, M1-P90, M1-A89, M1-G88, M1-E87, M1-E86, M1-A85, M1-E84, M1-T83, M1-E82, M1-G81, M1-H80, M1-A79, M1-G78, M1-R77, M1-T76, M1-G75, M1-R74, M1-A73, M1-E72, M1-A71, M1-E70, M1-P69, M1-G68, M1-R67, M1-D66, M1-P65, M1-G64, M1-G63, M1-G62, M1-G61, M1-A60, M1-E59, M1-K58, M1-V57, M1-A56, M1-A55, M1-A54, M1-G53, M1-R52, M1-P51, M1-A50, M1-E49, M1-E48, M1-A47, M1-G46, M1-E45, M1-S44, M1-G43, M1-E42, M1-P41, M1-G40, M1-E39, M1-A38, M1-E37, M1-G36, M1-G35, M1-A34, M1-A33, M1-G32, M1-P31, M1-E30, M1-G29, M1-P28, M1-R27, M1-E26, M1-A25, M1-L24, M1-P23, M1-A22, M1-P21, M1-V20, M1-E19, M1-P18, M1-P17, M1-G16, M1-Q15, M1-P14, M1-G13, M1-P12, M1-A11, M1-V10, M1-G9, M1-E8, and/or M1-P7 of SEQ ID NO: 2. Polynucleotide sequences encoding these polypeptides are also provided. The present invention also encompasses the use of these C-terminal HCLI deletion polypeptides as immunogenic and/or antigenic epitopes as described elsewhere herein.

Alternatively, preferred polypeptides of the present invention encompass polypeptide sequences corresponding to, for example, internal regions of the HCLI polypeptide (e.g., any combination of both N- and C-terminal HCLI polypeptide deletions) of SEQ ID NO: 2. For example, internal regions could be defined by the equation: amino acid NX to amino acid CX, wherein NX refers to any N-terminal deletion polypeptide amino acid of HCLI (SEQ ID NO: 2), and where CX refers to any C-terminal deletion polypeptide amino acid of HCLI (SEQ ID NO: 2). Polynucleotides encoding these polypeptides are also provided. The present invention also encompasses the use of these polypeptides as an immunogenic and/or antigenic epitope as describes elsewhere herein.

In preferred embodiments, the following N-terminal HCLI.v2 deletion polypeptides are encompassed by the present invention: M1-K686, A2-K686, E3-K686, A4-K686, A5-K686, E6-K686, P7-K686, E8-K686, G9-K686, V10-K686, A11-K686, P12-K686, G13-K686, P14-K686, Q15-K686, G16-K686, P17-K686, P18-K686, E19-K686, V20-K686, P21-K686, A22-K686, P23-K686, L24-K686, A25-K686, E26-K686, R27-K686, P28-K686, G29-K686, E30-K686, P31-K686, G32-K686, A33-K686, A34-K686, G35-K686, G36-K686, E37-K686, A38-K686, E39-K686, G40-K686, P41-K686, E42-K686, G43-K686, S44-K686, E45-K686, G46-K686, A47-K686, E48-K686, E49-K686, A50-K686, P51-K686, R52-K686, G53-K686, A54-K686, A55-K686, A56-K686, V57-K686, K58-K686, E59-K686, A60-K686, G61-K686, G62-K686, G63-K686, G64-K686, P65-K686, D66-K686, R67-K686, G68-K686, P69-K686, E70-K686, A71-K686, E72-K686, A73-K686, R74-K686, G75-K686, T76-K686, R77-K686, G78-K686, A79-K686, H80-K686, G81-K686, E82-K686, T83-K686, E84-K686, A85-K686, E86-K686, E87-K686, G88-K686, A89-K686, P90-K686, E91-K686, G92-K686, A93-K686, E94-K686, V95-K686, P96-K686, Q97-K686, G98-K686, G99-K686, E100-K686, E101-K686, T102-K686, S103-K686, G104-K686, A105-K686, Q106-K686, Q107-K686, V108-K686, E109-K686, G110-K686, A111-K686, S112-K686, P113-K686, G114-K686, R115-K686, G116-K686, A117-K686, Q118-K686, G119-K686, E120-K686, P121-K686, R122-K686, G123-K686, E124-K686, A125-K686, Q126-K686, R127-K686, E128-K686, P129-K686, E130-K686, D131-K686, S132-K686, A133-K686, A134-K686, P135-K686, E136-K686, R137-K686, Q138-K686, E139-K686, E140-K686, A141-K686, E142-K686, Q143-K686, R144-K686, P145-K686, E146-K686, V147-K686, P148-K686, E149-K686, G150-K686, S151-K686, A152-K686, S153-K686, G154-K686, E155-K686, A156-K686, G157-K686, D158-K686, S159-K686, V160-K686, D161-K686, A162-K686, E163-K686, G164-K686, P165-K686, L166-K686, G167-K686, D168-K686, N169-K686, I170-K686, E171-K686, A172-K686, E173-K686, G174-K686, P175-K686, A176-K686, G177-K686, D178-K686, S179-K686, V180-K686, E181-K686, A182-K686, E183-K686, G184-K686, R185-K686, V186-K686, G187-K686, D188-K686, S189-K686, V190-K686, D191-K686, A192-K686, E193-K686, G194-K686, P195-K686, A196-K686, G197-K686, D198-K686, S199-K686, V200-K686, D201-K686, A202-K686, E203-K686, G204-K686, P205-K686, L206-K686, G207-K686, D208-K686, N209-K686, I210-K686, Q211-K686, A212-K686, E213-K686, G214-K686, P215-K686, A216-K686, G217-K686, D218-K686, S219-K686, V220-K686, D221-K686, A222-K686, E223-K686, G224-K686, R225-K686, V226-K686, G227-K686, D228-K686, S229-K686, V230-K686, D231-K686, A232-K686, E233-K686, G234-K686, P235-K686, A236-K686, G237-K686, D238-K686, S239-K686, V240-K686, D241-K686, A242-K686, E243-K686, G244-K686, R245-K686, V246-K686, G247-K686, D248-K686, S249-K686, V250-K686, E251-K686, A252-K686, G253-K686, D254-K686, P255-K686, A256-K686, G257-K686, D258-K686, G259-K686, V260-K686, E261-K686, A262-K686, G263-K686, V264-K686, P265-K686, A266-K686, G267-K686, D268-K686, S269-K686, V270-K686, E271-K686, A272-K686, E273-K686, G274-K686, P275-K686, A276-K686, G277-K686, D278-K686, S279-K686, M280-K686, D281-K686, A282-K686, E283-K686, G284-K686, P285-K686, A286-K686, G287-K686, R288-K686, A289-K686, R290-K686, R291-K686, V292-K686, S293-K686, G294-K686, E295-K686, P296-K686, Q297-K686, Q298-K686, S299-K686, G300-K686, D301-K686, G302-K686, S303-K686, L304-K686, S305-K686, P306-K686, Q307-K686, A308-K686, E309-K686, A310-K686, I311-K686, E312-K686, V313-K686, A314-K686, A315-K686, G316-K686, E317-K686, S318-K686, A319-K686, G320-K686, R321-K686, S322-K686, P323-K686, G324-K686, E325-K686, L326-K686, A327-K686, W328-K686, D329-K686, A330-K686, A331-K686, E332-K686, E333-K686, A334-K686, E335-K686, V336-K686, P337-K686, G338-K686, V339-K686, K340-K686, G341-K686, S342-K686, E343-K686, E344-K686, A345-K686, A346-K686, P347-K686, G348-K686, D349-K686, A350-K686, R351-K686, A352-K686, D353-K686, A354-K686, G355-K686, E356-K686, D357-K686, R358-K686, V359-K686, G360-K686, D361-K686, G362-K686, P363-K686, Q364-K686, Q365-K686, E366-K686, P367-K686, G368-K686, E369-K686, D370-K686, E371-K686, E372-K686, R373-K686, R374-K686, E375-K686, R376-K686, S377-K686, P378-K686, E379-K686, G380-K686, P381-K686, R382-K686, E383-K686, E384-K686, E385-K686, A386-K686, A387-K686, G388-K686, G389-K686, E390-K686, E391-K686, E392-K686, S393-K686, P394-K686, D395-K686, S396-K686, S397-K686, P398-K686, H399-K686, G400-K686, E401-K686, A402-K686, S403-K686, R404-K686, G405-K686, A406-K686, A407-K686, E408-K686, P409-K686, E410-K686, A411-K686, Q412-K686, L413-K686, S414-K686, N415-K686, H416-K686, L417-K686, A418-K686, E419-K686, E420-K686, G421-K686, P422-K686, A423-K686, E424-K686, G425-K686, S426-K686, G427-K686, E428-K686, A429-K686, A430-K686, R431-K686, V432-K686, N433-K686, G434-K686, R435-K686, R436-K686, E437-K686, D438-K686, G439-K686, E440-K686, A441-K686, S442-K686, E443-K686, P444-K686, R445-K686, A446-K686, L447-K686, G448-K686, Q449-K686, E450-K686, H451-K686, D452-K686, I453-K686, T454-K686, L455-K686, F456-K686, V457-K686, K458-K686, A459-K686, G460-K686, Y461-K686, D462-K686, G463-K686, E464-K686, S465-K686, I466-K686, G467-K686, N468-K686, C469-K686, P470-K686, F471-K686, S472-K686, Q473-K686, R474-K686, L475-K686, F476-K686, M477-K686, I478-K686, L479-K686, W480-K686, L481-K686, K482-K686, G483-K686, V484-K686, I485-K686, F486-K686, N487-K686, V488-K686, T489-K686, T490-K686, V491-K686, D492-K686, L493-K686, K494-K686, R495-K686, K496-K686, P497-K686, A498-K686, D499-K686, L500-K686, Q501-K686, N502-K686, L503-K686, A504-K686, P505-K686, G506-K686, T507-K686, N508-K686, P509-K686, P510-K686, F511-K686, M512-K686, T513-K686, F514-K686, D515-K686, G516-K686, E517-K686, V518-K686, K519-K686, T520-K686, D521-K686, V522-K686, N523-K686, K524-K686, I525-K686, E526-K686, E527-K686, F528-K686, L529-K686, E530-K686, E531-K686, K532-K686, L533-K686, A534-K686, P535-K686, P536-K686, R537-K686, Y538-K686, P539-K686, K540-K686, L541-K686, G542-K686, T543-K686, Q544-K686, H545-K686, P546-K686, E547-K686, S548-K686, N549-K686, S550-K686, A551-K686, G552-K686, N553-K686, D554-K686, V555-K686, F556-K686, A557-K686, K558-K686, F559-K686, S560-K686, A561-K686, F562-K686, I563-K686, K564-K686, N565-K686, T566-K686, K567-K686, K568-K686, D569-K686, A570-K686, N571-K686, E572-K686, I573-K686, H574-K686, E575-K686, K576-K686, N577-K686, L578-K686, L579-K686, K580-K686, A581-K686, L582-K686, R583-K686, K584-K686, L585-K686, D586-K686, N587-K686, Y588-K686, L589-K686, N590-K686, S591-K686, P592-K686, L593-K686, P594-K686, D595-K686, E596-K686, I597-K686, D598-K686, A599-K686, Y600-K686, S601-K686, T602-K686, E603-K686, D604-K686, V605-K686, T606-K686, V607-K686, S608-K686, G609-K686, R610-K686, K611-K686, F612-K686, L613-K686, G614-K686, G615-K686, D616-K686, E617-K686, L618-K686, T619-K686, L620-K686, A621-K686, D622-K686, C623-K686, N624-K686, L625-K686, L626-K686, P627-K686, K6 628-K686, L629-K686, H630-K686, I631-K686, I632-K686, K633-K686, I634-K686, V635-K686, A636-K686, K637-K686, K638-K686, Y639-K686, R640-K686, D641-K686, F642-K686, E643-K686, F644-K686, P645-K686, S646-K686, E647-K686, M648-K686, T649-K686, G650-K686, I651-K686, W652-K686, R653-K686, Y654-K686, L655-K686, N656-K686, N657-K686, A658-K686, Y659-K686, A660-K686, R661-K686, D662-K686, E663-K686, F664-K686, T665-K686, N666-K686, T667-K686, C668-K686, P669-K686, A670-K686, D671-K686, Q672-K686, E673-K686, I674-K686, E675-K686, H676-K686, A677-K686, Y678-K686, S679-K686, and/or D680-K686 of SEQ ID NO: 17. Polynucleotide sequences encoding these polypeptides are also provided. The present invention also encompasses the use of these N-terminal HCLI.v2 deletion polypeptides as immunogenic and/or antigenic epitopes as described elsewhere herein.

In preferred embodiments, the following C-terminal HCLI.v2 deletion polypeptides are encompassed by the present invention: M1-K686, M1-M685, M1-R684, M1-K683, M1-A682, M1-V681, M1-D680, M1-S679, M1-Y678, M1-A677, M1-H676, M1-E675, M1-I674, M1-E673, M1-Q672, M1-D671, M1-A670, M1-P669, M1-C668, M1-T667, M1-N666, M1-T665, M1-F664, M1-E663, M1-D662, M1-R661, M1-A660, M1-Y659, M1-A658, M1-N657, M1-N656, M1-L655, M1-Y654, M1-R653, M1-W652, M1-I651, M1-G650, M1-T649, M1-M648, M1-E647, M1-S646, M1-P645, M1-F644, M1-E643, M1-F642, M1-D641, M1-R640, M1-Y639, M1-K638, M1-K637, M1-A636, M1-V635, M1-I634, M1-K633, M1-I632, M1-I631, M1-H630, M1-L629, M1-K628, M1-P627, M1-L626, M1-L625, M1-N624, M1-C623, M1-D622, M1-A621, M1-L620, M1-T619, M1-L618, M1-E617, M1-D616, M1-G615, M1-G614, M1-L613, M1-F612, M1-K611, M1-R610, M1-G609, M1-S608, M1-V607, M1-T606, M1-V605, M1-D604, M1-E603, M1-T602, M1-S601, M1-Y600, M1-A599, M1-D598, M1-I597, M1-E596, M1-D595, M1-P594, M1-L593, M1-P592, M1-S591, M1-N590, M1-L589, M1-Y588, M1-N587, M1-D586, M1-L585, M1-K584, M1-R583, M1-L582, M1-A581, M1-K580, M1-L579, M1-L578, M1-N577, M1-K576, M1-E575, M1-H574, M1-I573, M1-E572, M1-N571, M1-A570, M1-D569, M1-K568, M1-K567, M1-T566, M1-N565, M1-K564, M1-I563, M1-F562, M1-A561, M1-S560, M1-F559, M1-K558, M1-A557, M1-F556, M1-V555, M1-D554, M1-N553, M1-G552, M1-A551, M1-S550, M1-N549, M1-S548, M1-E547, M1-P546, M1-H545, M1-Q544, M1-T543, M1-G542, M1-L541, M1-K540, M1-P539, M1-Y538, M1-R537, M1-P536, M1-P535, M1-A534, M1-L533, M1-K532, M1-E531, M1-E530, M1-L529, M1-F528, M1-E527, M1-E526, M1-I525, M1-K524, M1-N523, M1-V522, M1-D521, M1-T520, M1-K519, M1-V518, M1-E517, M1-G516, M1-D515, M1-F514, M1-T513, M1-M512, M1-F511, M1-P510, M1-P509, M1-N508, M1-T507, M1-G506, M1-P505, M1-A504, M1L503, M1-N502, M1-Q501, M1-L500, M1-D499, M1-A498, M1-P497, M1-K496, M1-R495, M1-K494, M1-L493, M1-D492, M1-V491, M1-T490, M1-T489, M1-V488, M1-N487, M1-F486, M1-I485, M1-V484, M1-G483, M1-K482, M1-L481, M1-W480, M1-L479, M1-I478, M1-M477, M1-F476, M1-L475, M1-R474, M1-Q473, M1-S472, M1-F471, M1-P470, M1-C469, M1-N468, M1-G467, M1-I466, M1-S465, M1-E464, M1-G463, M1-D462, M1-Y461, M1-G460, M1-A459, M1-K458, M1-V457, M1-F456, M1-L455, M1-T454, M1-I453, M1-D452, M1-H451, M1-E450, M1-Q449, M1-G448, M1-L447, M1-A446, M1-R445, M1-P444, M1-E443, M1-S442, M1-A441, M1-E440, M1-G439, M1-D438, M1-E437, M1-R436, M1-R435, M1-G434, M1-N433, M1-V432, M1-R431, M1-A430, M1-A429, M1-E428, M1-G427, M1-S426, M1-G425, M1-E424, M1-A423, M1-P422, M1-G421, M1-E420, M1-E419, M1-A418, M1-L417, M1-H416, M1-N415, M1-S414, M1-L413, M1-Q412, M1-A411, M1-E410, M1-P409, M1-E408, M1-A407, M1-A406, M1-G405, M1-R404, M1-S403, M1-A402, M1-E401, M1-G400, M1-H399, M1-P398, M1-S397, M1-S396, M1-D395, M1-P394, M1-S393, M1-E392, M1-E391, M1-E390, M1-G389, M1-G388, M1-A387, M1-A386, M1-E385, M1-E384, M1-E383, M1-R382, M1-P381, M1-G380, M1-E379, M1-P378, M1-S377, M1-R376, M1-E375, M1-R374, M1-R373, M1-E372, M1-E371, M1-D370, M1-E369, M1-G368, M1-P367, M1-E366, M1-Q365, M1-Q364, M1-P363, M1-G362, M1-D361, M1-G360, M1-V359, M1-R358, M1-D357, M1-E356, M1-G355, M1-A354, M1-D353, M1-A352, M1-R351, M1-A350, M1-D349, M1-G348, M1-P347, M1-A346, M1-A345, M1-E344, M1-E343, M1-S342, M1-G341, M1-K340, M1-V339, M1-G338, M1-P337, M1-V336, M1-E335, M1-A334, M1-E333, M1-E332, M1-A331, M1-A330, M1-D329, M1-W328, M1-A327, M1-L326, M1-E325, M1-G324, M1-P323, M1-S322, M1-R321, M1-G320, M1-A319, M1-S318, M1-E317, M1-G316, M1-A315, M1-A314, M1-V313, M1-E312, M1-I311, M1-A310, M1-E309, M1-A308, M1-Q307, M1-P306, M1-S305, M1-L304, M1-S303, M1-G302, M1-D301, M1-G300, M1-S299, M1-Q298, M1-Q297, M1-P296, M1-E295, M1-G294, M1-S293, M1-V292, M1-R291, M1-R290, M1-A289, M1-R288, M1-G287, M1-A286, M1-P285, M1-G284, M1-E283, M1-A282, M1-D281, M1-M280, M1-S279, M1-D278, M1-G277, M1-A276, M1-P275, M1-G274, M1-E273, M1-A272, M1-E271, M1-V270, M1-S269, M1-D268, M1-G267, M1-A266, M1-P265, M1-V264, M1-G263, M1-A262, M1-E261, M1-V260, M1-G259, M1-D258, M1-G257, M1-A256, M1-P255, M1-D254, M1-G253, M1-A252, M1-E251, M1-V250, M1-S249, M1-D248, M1-G247, M1-V246, M1-R245, M1-G244, M1-E243, M1-A242, M1-D241, M1-V240, M1-S239, M1-D238, M1-G237, M1-A236, M1-P235, M1-G234, M1-E233, M1-A232, M1-D231, M1-V230, M1-S229, M1-D228, M1-G227, M1-V226, M1-R225, M1-G224, M1-E223, M1-A222, M1-D221, M1-V220, M1-S219, M1-D218, M1-G217, M1-A216, M1-P215, M1-G214, M1-E213, M1-A212, M1-Q211, M1-I210, M1-N209, M1-D208, M1-G207, M1-L206, M1-P205, M1-G204, M1-E203, M1-A202, M1-D201, M1-V200, M1-S199, M1-D198, M1-G197, M1-A196, M1-P195, M1-G194, M1-E193, M1-A192, M1-D191, M1-V190, M1-S189, M1-D188, M1-G187, M1-V186, M1-R185, M1-G184, M1-E183, M1-A182, M1-E181, M1-V180, M1-S179, M1-D178, M1-G177, M1-A176, M1-P175, M1-G174, M1-E173, M1-A172, M1-E171, M1-I170, M1-N169, M1-D168, M1-G167, M1-L166, M1-P165, M1-G164, M1-E163, M1-A162, M1-D161, M1-V160, M1-S159, M1-D158, M1-G157, M1-A156, M1-E155, M1-G154, M1-S153, M1-A152, M1-S151, M1-G150, M1-E149, M1-P148, M1-V147, M1-E146, M1-P145, M1-R144, M1-Q143, M1-E142, M1-A141, M1-E140, M1-E139, M1-Q138, M1-R137, M1-E136, M1-P135, M1-A134, M1-A133, M1-S132, M1-D131, M1-E130, M1-P129, M1-E128, M1-R127, M1-Q126, M1-A125, M1-E124, M1-G123, M1-R122, M1-P121, M1-E120, M1-G119, M1-Q118, M1-A117, M1-G116, M1-R115, M1-G114, M1-P113, M1-S112, M1-A111, M1-G110, M1-E109, M1-V108, M1-Q107, M1-Q106, M1-A105, M1-G104, M1-S103, M1-T102, M1-E101, M1-E100, M1-G99, M1-G98, M1-Q97, M1-P96, M1-V95, M1-E94, M1-A93, M1-G92, M1-E91, M1-P90, M1-A89, M1-G88, M1-E87, M1-E86, M1-A85, M1-E84, M1-T83, M1-E82, M1-G81, M1-H80, M1-A79, M1-G78, M1-R77, M1-T76, M1-G75, M1-R74, M1-A73, M1-E72, M1-A71, M1-E70, M1-P69, M1-G68, M1-R67, M1-D66, M1-P65, M1-G64, M1-G63, M1-G62, M1-G61, M1-A60, M1-E59, M1-K58, M1-V57, M1-A56, M1-A55, M1-A54, M1-G53, M1-R52, M1-P51, M1-A50, M1-E49, M1-E48, M1-A47, M1-G46, M1-E45, M1-S44, M1-G43, M1-E42, M1-P41, M1-G40, M1-E39, M1-A38, M1-E37, M1-G36, M1-G35, M1-A34, M1-A33, M1-G32, M1-P31, M1-E30, M1-G29, M1-P28, M1-R27, M1-E26, M1-A25, M1-L24, M1-P23, M1-A22, M1-P21, M1-V20, M1-E19, M1-P18, M1-P17, M1-G16, M1-Q15, M1-P14, M1-G13, M1-P12, M1-A11, M1-V10, M1-G9, M1-E8, and/or M1-P7 of SEQ ID NO: 17. Polynucleotide sequences encoding these polypeptides are also provided. The present invention also encompasses the use of these C-terminal HCLI.v2 deletion polypeptides as immunogenic and/or antigenic epitopes as described elsewhere herein.

Alternatively, preferred polypeptides of the present invention encompass polypeptide sequences corresponding to, for example, internal regions of the HCLI.v2 polypeptide (e.g., any combination of both N- and C-terminal HCLI.v2 polypeptide deletions) of SEQ ID NO: 17. For example, internal regions could be defined by the equation: amino acid NX to amino acid CX, wherein NX refers to any N-terminal deletion polypeptide amino acid of HCLI.v2 (SEQ ID NO: 17), and where CX refers to any C-terminal deletion polypeptide amino acid of HCLI.v2 (SEQ ID NO: 17). Polynucleotides encoding these polypeptides are also provided. The present invention also encompasses the use of these polypeptides as an immunogenic and/or antigenic epitope as describes elsewhere herein.

In another embodiment, the present invention encompasses a polynucleotide lacking the initiating start codon, in addition to, the resulting encoded polypeptide of HCLI. Specifically, the present invention encompasses the polynucleotide corresponding to nucleotides 4 through 1887 of SEQ ID NO: 1, and the polypeptide corresponding to amino acids 2 through 629 of SEQ ID NO: 2. Also encompassed are recombinant vectors comprising the encoding sequence, and host cells comprising the vector.

In another embodiment, the present invention encompasses a polynucleotide lacking the initiating start codon, in addition to, the resulting encoded polypeptide of HCLI.v2. Specifically, the present invention encompasses the polynucleotide corresponding to nucleotides 4 through 2058 of SEQ ID NO: 16, and the polypeptide corresponding to amino acids 2 through 686 of SEQ ID NO: 17. Also encompassed are recombinant vectors comprising the encoding sequence, and host cells comprising the vector.

Although nucleic acid sequences which encode the HCLI polypeptide and variants thereof are preferably capable of hybridizing to the nucleotide sequence of the naturally occurring HCLI polypeptide under appropriately selected conditions of stringency, it may be advantageous to produce nucleotide sequences encoding HCLI polypeptides, or derivatives thereof, which possess a substantially different codon usage. For example, codons may be selected to increase the rate at which expression of the peptide/polypeptide occurs in a particular prokaryotic or eukaryotic host in accordance with the frequency with which particular codons are utilized by the host. Another reason for substantially altering the nucleotide sequence encoding an HCLI polypeptide, or its derivatives, without altering the encoded amino acid sequences, includes the production of RNA transcripts having more desirable properties, such as a greater half-life, than transcripts produced from the naturally occurring sequence.

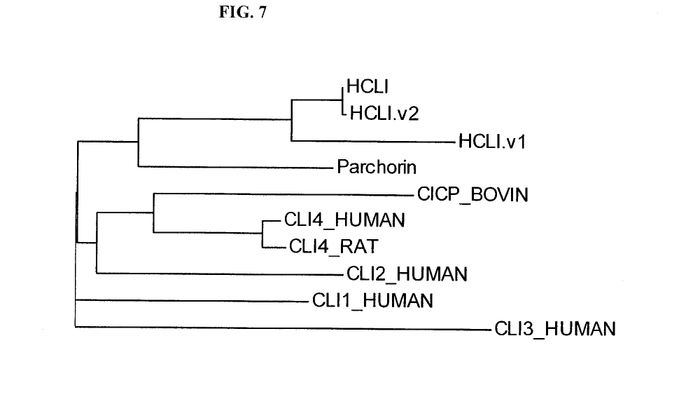

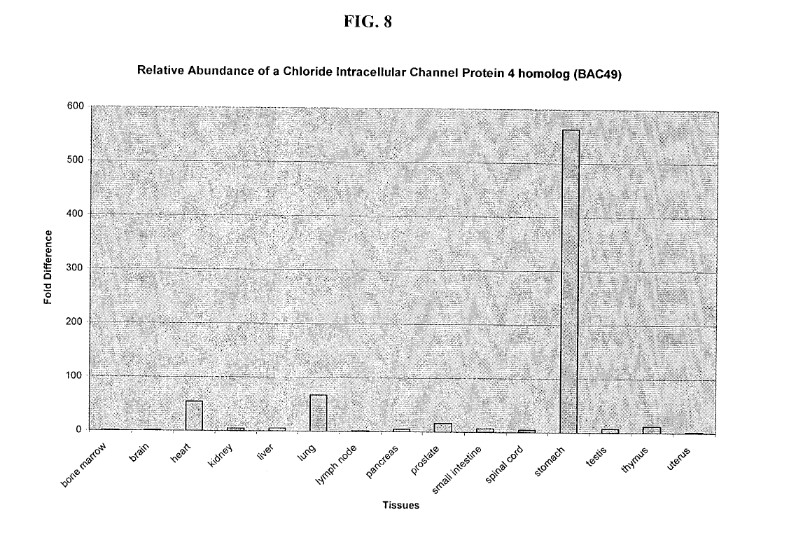

Expression profiling designed to measure the steady state mRNA levels encoding the HCLI polypeptide showed predominately high expression levels in stomach; significantly in heart, lung, and to a lesser extent, in other tissues as shown in FIG. 8.

Expanded analysis of HCLI expression levels by TaqMan™ quantitative PCR (see FIG. 9) confirmed that the HCLI polypeptide is expressed in vascular tissues (FIG. 8). HCLI mRNA was expressed predominately in the blood vessel choroid plexus. Significant expression was observed in stomach, primary and tertiary bronchus of the lung, the liver, and to a lesser extent in the gallbladder.

The predominate expression in choroid plexus is consistent with HCLI representing a novel chloride channel protein. The closest homolog of HCLI, the parchorin polypeptide, is a chloride channel protein that is highly enriched in tissues that secrete water, such as parietal cells, choroid plexus, salivary duct, lacrimal gland, kidney, and airway epithelial tissues (Nishizawa, T. et al., J. Biol. Chem., 275:11164-11173, (2000)). As a result, it is possible that HCLI and parchorin at least share some biological activity, and likely work coordinately in controlling normal chloride homeostasis, particularly in water secreting tissues, amongst others.

The choroid plexus region of the brain is thought to play integral roles in neuroprotection, and control the influx and efflux of drugs and metabolites (J Drug Target. June 2002;10(4):353-7); Microsc Res Tech. Jan. 1, 2001;52(1):83-8). Additionally, the choroid plexus is also the site of several disorders, particularly choroid plexus tumors, papillomas (Microsc Res Tech. Jan. 1, 2001;52(1):104-11), and is thought to be involved in central nervous system inflammation (Microsc Res Tech. Jan. 1, 2001;52(1):112-29). The choroid plexus also plays role in the transport of cerebral spinal fluid, and also controls the concentration of glucose and amino acids in cerebral spinal fluid (Microsc Res Tech Jan. 1, 2001;52(1):38-48). In addition, the choroid plexus is also the site of synthesis for the transthyretin protein, which is involved in the transport of thyroid hormones form the blood to the brain tissues (Microsc Res Tech Jan. 1, 2001;52(1):21-30). Thyroid hormones are key regulators of brain differentiation and function.

Based on the above, HCLI polynucleotides and polypeptides may be useful in treating, diagnosing, prognosing, and/or preventing disorders associated with aberrant cerebral spinal fluid synthesis, aberrant control of cerebral spinal fluid volume, aberrant composition of cerebral spinal fluid, aberrant neuroprotection, choroid plexus tumors, choroid plexus papillomas, disorders associated with aberrant central nervous system inflammation, aberrant transthyretin synthesis, aberrant transthyretin expression, aberrant transthyretin activity, aberrant brain differentiation, and/or aberrant brain function.

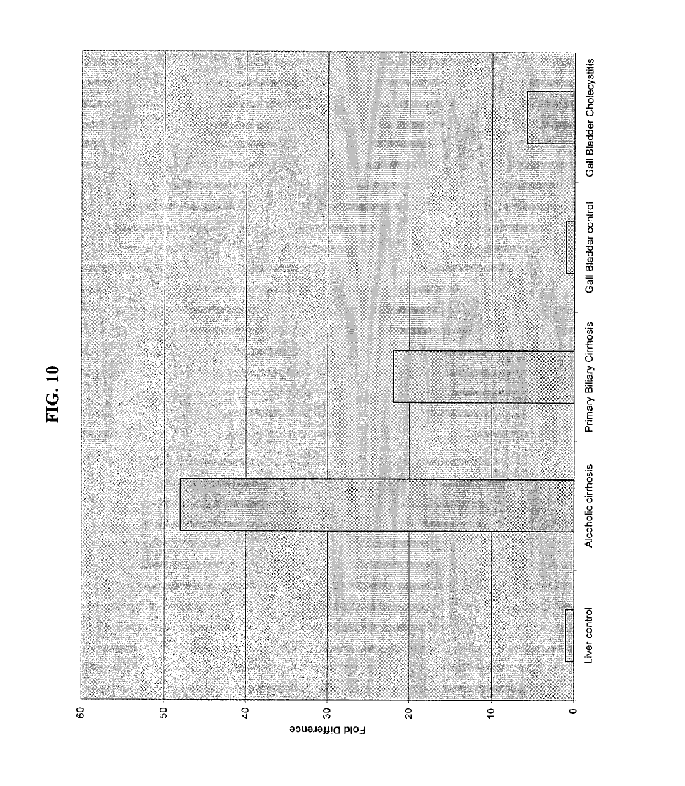

Morever, an additional analysis of HCLI expression levels by TaqMan™ quantitative PCR (see FIG. 10) in disease cells and tissues indicated that the HCLI polypeptide is differentially expressed in alcoholic cirrhosis, and gall bladder cholecystitis diseased tissues. In the alcoholic cirrhosis disease tissue results, one sample showed an approximately 48-fold induction in HCLI steady state RNA over that observed in one normal sample. HCLI may participate in the inflammatory process in the etiology of liver cirrhosis and small molecule modulators of HCLI function may represent a novel therapeutic option in the treatment of chronic liver diseases, particularly liver cirrhosis.

In the gall bladder cholecystitis tissue results, differential expression of HCLI in gall bladder cholecystitis tissue was observed with an approximately 5-fold higher level of expression as compared to one control. Therefore, HCLI modulators, which include, for example, small molecule and biological antagonists of HCLI, may provide a novel and specific treatment for metabolic disorders of the gall bladder, particularly gall bladder cholecystitis.

The strong homology to intracellular chloride ion channels, combined with the differential expression in alcoholic cirrhosis and gall bladder cholecystitis tissue suggests the HCLI polynucleotides and polypeptides may be useful in treating, diagnosing, prognosing, and/or preventing cardiovascular diseases and/or disorders, which include, but are not limited to: altered chloride/ion homeostasis, particularly in the choroid plexus such as hyponatremia, and hypernatremia, in the lung such as cystic fibrosis, the liver such as cirrhosis and the gall bladder such as cholecystitis.

The strong homology to intracellular chloride ion channels, combined with the differential expression in alcoholic liver cirrhosis tissue also suggests the potential utility for HCLI polynucleotides and polypeptides in treating, diagnosing, prognosing, and/or preventing hepatic disorders. Briefly, the protein can be used for the detection, treatment, amelioration, and/or prevention of hepatoblastoma, jaundice, hepatitis, liver metabolic diseases and conditions that are attributable to the differentiation of hepatocyte progenitor cells, cirrhosis, hepatic cysts, pyrogenic abscess, amebic abcess, hydatid cyst, cystadenocarcinoma, adenoma, focal nodular hyperplasia, hemangioma, hepatocellulae carcinoma, cholangiocarcinoma, and angiosarcoma, granulomatous liver disease, liver transplantation, hyperbilirubinemia, jaundice, parenchymal liver disease, portal hypertension, hepatobiliary disease, hepatic parenchyma, hepatic fibrosis, anemia, gallstones, cholestasis, carbon tetrachloride toxicity, beryllium toxicity, vinyl chloride toxicity, choledocholithiasis, hepatocellular necrosis, aberrant metabolism of amino acids, aberrant metabolism of carbohydrates, aberrant synthesis proteins, aberrant synthesis of glycoproteins, aberrant degradation of proteins, aberrant degradation of glycoproteins, aberrant metabolism of drugs, aberrant metabolism of hormones, aberrant degradation of drugs, aberrant degradation of drugs, aberrant regulation of lipid metabolism, aberrant regulation of cholesterol metabolism, aberrant glycogenesis, aberrant glycogenolysis, aberrant glycolysis, aberrant gluconeogenesis, hyperglycemia, glucose intolerance, hyperglycemia, decreased hepatic glucose uptake, decreased hepatic glycogen synthesis, hepatic resistance to insulin, portal-systemic glucose shunting, peripheral insulin resistance, hormonal abnormalities, increased levels of systemic glucagon, decreased levels of systemic cortisol, increased levels of systemic insulin, hypoglycemia, decreased gluconeogenesis, decreased hepatic glycogen content, hepatic resistance to glucagon, elevated levels of systemic aromatic amino acids, decreased levels of systemic branched-chain amino acids, hepatic encephalopathy, aberrant hepatic amino acid transamination, aberrant hepatic amino acid oxidative deamination, aberrant ammonia synthesis, aberant albumin secretion, hypoalbuminemia, aberrant cytochromes b5 function, aberrant P450 function, aberrant glutathione S-acyltransferase function, aberrant cholesterol synthesis, and aberrant bile acid synthesis.

Moreover, polynucleotides and polypeptides, including fragments and/or antagonists thereof, have uses which include, directly or indirectly, treating, preventing, diagnosing, and/or prognosing the following, non-limiting, hepatic infections: liver disease caused by sepsis infection, liver disease caused by bacteremia, liver disease caused by Pneomococcal pneumonia infection, liver disease caused by Toxic shock syndrome, liver disease caused by Listeriosis, liver disease caused by Legionnaries' disease, liver disease caused by Brucellosis infection, liver disease caused by Neisseria gonorrhoeae infection, liver disease caused by Yersinia infection, liver disease caused by Salmonellosis, liver disease caused by Nocardiosis, liver disease caused by Spirochete infection, liver disease caused by Treponema pallidum infection, liver disease caused by Brrelia burgdorferi infection, liver disease caused by Leptospirosis, liver disease caused by Coxiella burnetii infection, liver disease caused by Rickettsia richettsii infection, liver disease caused by Chlamydia trachomatis infection, liver disease caused by Chlamydia psittaci infection, liver disease caused by hepatitis virus infection, liver disease caused by Epstein-Barr virus infection in addition to any other hepatic disease and/or disorder implicated by the causative agents listed above or elsewhere herein.

The strong homology to intracellular chloride ion channels, combined with the predominate localized expression in heart tissue suggests the HCLI polynucleotides and polypeptides may be useful in treating, diagnosing, prognosing, and/or preventing cardiovascular diseases and/or disorders, which include, but are not limited to: myocardio infarction, congestive heart failure, arrthymias, cardiomyopathy, atherosclerosis, arterialsclerosis, microvascular disease, embolism, thromobosis, pulmonary edema, palpitation, dyspnea, angina, hypotension, syncope, heart murmer, aberrant ECG, hypertrophic cardiomyopathy, the Marfan syndrome, sudden death, prolonged QT syndrome, congenital defects, cardiac viral infections, valvular heart disease, and hypertension.

Similarly, HCLI polynucleotides and polypeptides may be useful for ameliorating cardiovascular diseases and symptoms which result indirectly from various non-cardiavascular effects, which include, but are not limited to, the following, obesity, smoking, Down syndrome (associated with endocardial cushion defect); bony abnormalities of the upper extremities (associated with atrial septal defect in the Holt-Oram syndrome); muscular dystrophies (associated with cardiomyopathy); hemochromatosis and glycogen storage disease (associated with myocardial infiltration and restrictive cardiomyopathy); congenital deafness (associated with prolonged QT interval and serious cardiac arrhythmias); Raynaud's disease (associated with primary pulmonary hypertension and coronary vasospasm); connective tissue disorders, i.e., the Marfan syndrome, Ehlers-Danlos and Hurler syndromes, and related disorders of mucopolysaccharide metabolism (aortic dilatation, prolapsed mitral valve, a variety of arterial abnormalities); acromegaly (hypertension, accelerated coronary atherosclerosis, conduction defects, cardiomyopathy); hyperthyroidism (heart failure, atrial fibrillation); hypothyroidism (pericardial effusion, coronary artery disease); rheumatoid arthritis (pericarditis, aortic valve disease); scleroderma (cor pulmonale, myocardial fibrosis, pericarditis); systemic lupus erythematosus (valvulitis, myocarditis, pericarditis); sarcoidosis (arrhythmias, cardiomyopathy); postmenopausal effects, Chlamydial infections, polycystic ovary disease, thyroid disease, alcoholism, diet, and exfoliative dermatitis (high-output heart failure), for example.

Moreover, polynucleotides and polypeptides, including fragments and/or antagonists thereof, have uses which include, directly or indirectly, treating, preventing, diagnosing, and/or prognosing the following, non-limiting, cardiovascular infections: blood stream invasion, bacteremia, sepsis, Streptococcus pneumoniae infection, group a streptococci infection, group b streptococci infection, Enterococcus infection, nonenterococcal group D streptococci infection, nonenterococcal group C streptococci infection, nonenterococcal group G streptococci infection, Streptoccus viridans infection, Staphylococcus aureus infection, coagulase-negative staphylococci infection, gram-negative Bacilli infection, Enterobacteriaceae infection, Psudomonas spp. Infection, Acinobacter spp. Infection, Flavobacterium meningosepticum infection, Aeromonas spp. Infection, Stenotrophomonas maltophilia infection, gram-negative coccobacilli infection, Haemophilus influenza infection, Branhamella catarrhalis infection, anaerobe infection, Bacteriodes fragilis infection, Clostridium infection, fungal infection, Candida spp. Infection, non-albicans Candida spp. Infection, Hansenula anomala infection, Malassezia furfur infection, nontuberculous Mycobacteria infection, Mycobacterium avium infection, Mycobacterium chelonae infection, Mycobacterium fortuitum infection, spirochetal infection, Borrelia burgdorferi infection, in addition to any other cardiovascular disease and/or disorder (e.g., non-sepsis) implicated by the causative agents listed above or elsewhere herein.

Moreover, the HCL polynucleotide, polypeptides, variants thereof, fragments thereof, and/or modulators thereof, are useful for the treatment, amelioration, and/or detection of vascular disorders and conditions, which include, but are not limited to miscrovascular disease, vascular leak syndrome, aneurysm, stroke, embolism, thrombosis, coronary artery disease, arteriosclerosis, and/or atherosclerosis. Furthermore, the protein may also be used to determine biological activity, raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

The present invention also encompasses the production of DNA sequences, or portions thereof, which encode the HCLI polypeptide, or derivatives thereof, entirely by synthetic chemistry. After production, the synthetic sequence may be inserted into any of the many available expression vectors and cell systems using reagents that are well known and practiced by those in the art. Moreover, synthetic chemistry may be used to introduce mutations into a sequence encoding an HCLI polypeptide, or any fragment thereof.

In an embodiment of the present invention, a polynucleotide delivery vector containing the polynucleotide, or functional fragment thereof is provided. Preferably, the polynucleotide delivery vector contains the polynucleotide, or functional fragment thereof comprising an isolated and purified polynucleotide encoding a human HCLI having the sequence as set forth in SEQ ID NO: 1.

It will also be appreciated by those skilled in the pertinent art that in addition to the primers disclosed in SEQ ID NOS: 12-14, a longer oligonucleotide probe, or mixtures of probes, for example, depolynucleotiderate probes, can be used to detect longer, or more complex, nucleic acid sequences, such as, for example, genomic or full length DNA. In such cases, the probe may comprise at least 20-300 nucleotides, preferably, at least 30-100 nucleotides, and more preferably, 50-100 nucleotides.

The present invention also provides methods used to obtain the sequence of the HCLI polynucleotide and thus polypeptide as described herein. In one instance, the method of multiplex cloning was devised as a means of extending large numbers of bioinformatic polynucleotide predictions into full length sequences by multiplexing probes and cDNA libraries in an effort to minimize the overall effort typically required for cDNA cloning. The method relies on the conversion of plasmid-based, directionally cloned cDNA libraries into a population of pure, covalently-closed, circular, single-stranded molecules and long biotinylated DNA oligonucleotide probes designed from predicted polynucleotide sequences.

For such a multiplex cloning method, (see, for example, Example 3 herein), probes and libraries were subjected to solution hybridization in a formamide buffer which has been found to be superior to aqueous buffers typically used in other biotin/streptavidin cDNA capture methods (e.g., GeneTrapper). The hybridization was performed without prior knowledge of the clones represented in the libraries. Hybridization was performed two times. After the first selection, the isolated sequences were screened with PCR primers specific for the targeted clones. The second hybridization was carried out with only those oligo probes whose polynucleotide-specific PCR assays gave positive results.

The secondary hybridization serves to ‘normalize’ the selected library, thereby decreasing the amount of screening needed to identify particular clones. The method is robust and sensitive. Typically, dozens of cDNAs are isolated for any one particular polynucleotide, thereby increasing the chances of obtaining a full length cDNA. The entire complexity of any cDNA library is screened in the solution hybridization process, which is advantageous for finding rare sequences. The procedure is scalable, with 50 oligonucleotide probes per experiment currently being used, although this is not to be considered a limiting number.

Using bioinformatic predicted polynucleotide sequence, the following types of PCR primers and cloning oligos can be designed: A) PCR primer pairs that reside within a single predicted exon; B) PCR primer pairs that cross putative exon/intron boundaries; and C) 80 mer antisense and sense oligos containing a biotin moiety on the 5′ end. The primer pairs of the A type above are optimized on human genomic DNA; the B type primer pairs are optimized on a mixture of first strand cDNAs made with and without reverse transcriptase. Primers are optimized using mRNA derived from appropriate tissues sources, in this case, brain and testis poly A+ RNA was used.

The information obtained with the B type primers is used to assess those putative expressed sequences which can be experimentally observed to have reverse transcriptase-dependent expression. The primer pairs of the A type are less stringent in terms of identifying expressed sequences. However, because they amplify genomic DNA as well as cDNA, their ability to amplify genomic DNA provides for the necessary positive control for the primer pair. Negative results with the B type are subject to the caveat that the sequence(s) may not be expressed in the tissue first strand that is under examination.

The biotinylated 80-mer oligonucleotides are added en mass to pools of single strand cDNA libraries. Up to 50 probes have been successfully used on pools for 15 different libraries. After the primary selection is performed, all of the captured DNA is repaired to double strand form using the T7 primer for the commercial libraries in pCMVSPORT, and the SP6 primer for other constructed libraries in pSPORT. The resulting DNA is electroporated into E. coli DH12S and plated onto 150 mm plates with nylon filters. The cells are scraped and a frozen stock is made, thereby comprising the primary selected library.

One-fifth of the library is polynucleotidely converted into single strand form and the DNA is assayed with polynucleotide specific primer pairs (GSPs). The next round of solution hybridization capture is carried out with 80 mer oligos for only those sequences that are positive with the polynucleotide-specific-primers. After the second round, the captured single strand DNAs are repaired with a pool of GSPs, where only the primer complementary to polarity of the single-stranded circular DNA is used (i.e., the antisense primer for pCMVSPORT and pSPORT1 and the sense primer for pSPORT2).

The resulting colonies are screened by PCR using the GSPs. Typically, greater than 80% of the clones are positive for any given GSP. The entire 96 well block of clones is subjected to “mini-prep”, as known in the art, and each of clones is sized by either PCR or restriction enzyme digestion. A selection of different sized clones for each targeted sequence is chosen for transposon-hopping and DNA sequencing.

Preferably, as for established cDNA cloning methods used by the skilled practitioner, the libraries employed are of high quality. High complexity and large average insert size are optimal. High Pressure Liquid Chromatography (HPLC) may be employed as a means of fractionating cDNA for the purpose of constructing libraries.

Another embodiment of the present invention provides a method of identifying full-length polynucleotides encoding the disclosed polypeptide. The HCLI polynucleotide of the present invention, the polynucleotide encoding the HCLI polypeptide of the present invention, or the polypeptide encoded by the deposited clone(s) preferably represent the complete coding region (i.e., full-length polynucleotide).

Several methods are known in the art for the identification of the 5′ or 3′ non-coding and/or coding portions of a given polynucleotide. The methods described herein are exemplary and should not be construed as limiting the scope of the invention. These methods include, but are not limited to, filter probing, clone enrichment using specific probes, and protocols similar or identical to 5′ and 3′ “RACE” protocols that are well known in the art. For instance, a method similar to 5′ RACE is available for polynucleotiderating the missing 5′ end of a desired full-length transcript. (Fromont-Racine et al., Nucleic Acids Res. 21(7):1683-1684 (1993)).

Briefly, in the RACE method, a specific RNA oligonucleotide is ligated to the 5′ ends of a population of RNA presumably containing full-length polynucleotide RNA transcripts. A primer set containing a primer specific to the ligated RNA oligonucleotide and a primer specific to a known sequence of the polynucleotide of interest is used to PCR amplify the 5′ portion of the desired full-length polynucleotide. This amplified product may then be sequenced and used to polynucleotiderate the full-length polynucleotide.

The above method utilizes total RNA isolated from the desired source, although poly-A+ RNA can be used. The RNA preparation is treated with phosphatase, if necessary, to eliminate 5′ phosphate groups on degraded or damaged RNA that may interfere with the later RNA ligase step. The phosphatase is preferably inactivated and the RNA is treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

The above-described modified RNA preparation is used as a template for first strand cDNA synthesis employing a polynucleotide specific oligonucleotide. The first strand synthesis reaction is used as a template for PCR amplification of the desired 5′ end using a primer specific to the ligated RNA oligonucleotide and a primer specific to the known sequence of the polynucleotide of interest. The resultant product is then sequenced and analyzed to confirm that the 5′ end sequence belongs to the desired polynucleotide. It may also be advantageous to optimize the RACE protocol to increase the probability of isolating additional 5′ or 3′ coding or non-coding sequences. Various methods of optimizing a RACE protocol are known in the art; for example, a detailed description summarizing these methods can be found in B. C. Schaefer, Anal. Biochem., 227:255-273, (1995).

An alternative method for carrying out 5′ or 3′ RACE for the identification of coding or non-coding nucleic acid sequences is provided by Frohman, M. A., et al., Proc. Nat'l. Acad. Sci. USA, 85:8998-9002 (1988). Briefly, a cDNA clone missing either the 5′ or 3′ end can be reconstructed to include the absent base pairs extending to the translational start or stop codon, respectively. In some cases, cDNAs are missing the start of translation for an encoded product. A brief description of a modification of the original 5′ RACE procedure is as follows. Poly A+ or total RNA is reverse transcribed with Superscript II (Gibco/BRL) and an antisense or an I complementary primer specific to any one of the cDNA sequences provided as SEQ ID NOS: 1 and 3. The primer is removed from the reaction with a Microcon Concentrator (Amicon). The first-strand cDNA is then tailed with dATP and terminal deoxynucleotide transferase (Gibco/BRL). Thus, an anchor sequence is produced which is needed for PCR amplification. The second strand is synthesized from the dA-tail in PCR buffer, Taq DNA polymerase (Perkin-Elmer Cetus), an oligo-dT primer containing three adjacent restriction sites (XhoI SalI and ClaI) at the 5′ end and a primer containing just these restriction sites. This double-stranded cDNA is PCR amplified for 40 cycles with the same primers, as well as a nested cDNA-specific antisense primer. The PCR products are size-separated on an ethidium bromide-agarose gel and the region of gel containing cDNA products having the predicted size of missing protein-coding DNA is removed.

cDNA is purified from the agarose with the Magic PCR Prep kit (Promega), restriction digested with XhoI or SalI, and ligated to a plasmid such as pBluescript SKII (Stratapolynucleotide) at XhoI and EcoRV sites. This DNA is transformed into bacteria and the plasmid clones sequenced to identify the correct protein-coding inserts. Correct 5′ ends are confirmed by comparing this sequence with the putatively identified homologue and overlap with the partial cDNA clone. Similar methods known in the art and/or commercial kits are used to amplify and recover 3′ ends.

Several quality-controlled kits are commercially available for purchase. Similar reagents and methods to those above are supplied in kit form from Gibco/BRL for both 5′ and 3′ RACE for recovery of full length polynucleotides. A second kit is available from Clontech which is a modification of a related technique, called single-stranded ligation to single-stranded cDNA, (SLIC), developed by Dumas et al., Nucleic Acids Res., 19:5227-32(1991). The major difference in the latter procedure is that the RNA is alkaline hydrolyzed after reverse transcription and RNA ligase is used to join a restriction site-containing anchor primer to the first-strand cDNA. This obviates the necessity for the dA-tailing reaction which results in a polyT stretch that can impede sequencing.

An alternative to polynucleotiderating 5′ or 3′ cDNA from RNA is to use cDNA library double-stranded DNA. An asymmetric PCR-amplified antisense cDNA strand is synthesized with an antisense cDNA-specific primer and a plasmid-anchored primer. These primers are removed and a symmetric PCR reaction is performed with a nested cDNA-specific antisense primer and the plasmid-anchored primer.

Also encompassed by the present invention are polynucleotide sequences that are capable of hybridizing to the novel HCLI nucleic acid sequence as set forth in SEQ ID NO: 1 under various conditions of stringency. Hybridization conditions are typically based on the melting temperature (Tₘ) of the nucleic acid binding complex or probe (see, G. M. Wahl and S. L. Berger, 1987; Methods Enzymol., 152:399-407 and A. R. Kimmel, 1987; Methods of Enzymol., 152:507-511), and may be used at a defined stringency. For example, included in the present invention are sequences capable of hybridizing under moderately stringent conditions to the HCLI sequence of SEQ ID NO: 1 and other sequences which are depolynucleotiderate to those which encode the novel HCLI polypeptide. For example, a non-limiting example of moderate stringency conditions include prewashing solution of 2×SSC, 0.5% SDS, 1.0 mM EDTA, pH 8.0, and hybridization conditions of 50° C., 5×SSC, overnight.

The nucleic acid sequence encoding the HCLI protein of the present invention may be extended by utilizing a partial nucleotide sequence and employing various methods known in the art to detect upstream sequences such as promoters and regulatory elements. For example, one method that can be employed is restriction-site PCR, which utilizes universal primers to retrieve unknown sequence adjacent to a known locus (See, e.g., G. Sarkar, 1993, PCR Methods Applic., 2:318-322). In particular, genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region or sequence (T. Triglia et al., 1988, Nucleic Acids Res., 16:8186). The primers may be designed using OLIGO 4.06 Primer Analysis software (National Biosciences, Inc., Plymouth, Minn.), or another appropriate program, to be 22-30 nucleotides in length, to have a GC content of 50% or more, and to anneal to the target sequence at temperatures about 68° C.-72° C. The method uses several restriction enzymes to polynucleotiderate a suitable fragment in the known region of a polynucleotide. The fragment is then circularized by intramolecular ligation and used as a PCR template.

Another method which may be used to amplify or extend sequences is capture PCR which involves PCR amplification of DNA fragments adjacent to a known sequence in human and yeast artificial chromosome (YAC) DNA (M. Lagerstrom et al., 1991, PCR Methods Applic., 1:111-119). In this method, multiple restriction enzyme digestions and ligations may also be used to place an engineered double-stranded sequence into an unknown portion of the DNA molecule before performing PCR. J. D. Parker et al. (1991; Nucleic Acids Res., 19:3055-3060) provide another method which may be used to retrieve unknown sequences. Bacterial artificial chromosomes (BACs) are also used for such applications. In addition, PCR, nested primers, and PROMOTERFINDER libraries can be used to “walk” genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are also preferable, since such libraries will contain more sequences that comprise the 5′ regions of polynucleotides. The use of a randomly primed library may be especially preferable for situations in which an oligo d(T) library does not yield a full-length cDNA. Genomic libraries may be useful for extension of sequence into the 5′ and 3′ non-transcribed regulatory regions.

The embodiments of the present invention can be practiced using methods for DNA sequencing which are well known and polynucleotidely available in the art. The methods may employ such enzymes as the Klenow fragment of DNA polymerase I, SEQUENASE (U.S. Biochemical Corp. Cleveland, Ohio), Taq polymerase (PE Biosystems), thermostable T7 polymerase (Amersham Pharmacia Biotech, Piscataway, N.J.), or combinations of recombinant polymerases and proofreading exonucleases such as the ELONGASE Amplification System marketed by Life Technologies (Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) and the ABI Catalyst and 373 and 377 DNA sequencers (PE Biosystems). Commercially available capillary electrophoresis systems may be used to analyze the size or confirm the nucleotide sequence of sequencing or PCR products. Capillary electrophoresis is especially preferable for the sequencing of small pieces of DNA, which might be present in limited amounts in a particular sample.

In another embodiment of the present invention, polynucleotide sequences or portions thereof which encode an HCLI polypeptide or peptides can comprise recombinant DNA molecules to direct the expression of HCLI polypeptide products, peptide fragments, or functional equivalents thereof, in appropriate host cells. The HCLI polypeptides and peptides can be used for the polynucleotideration of specific antibodies, as described herein, and as bait in yeast two hybrid screens (and in other protein-protein interaction screens) to identify proteins that specifically interact with HCLI. Because of the inherent depolynucleotideracy of the polynucleotide code, other DNA sequences, which encode substantially the same or a functionally equivalent amino acid sequence, may be produced and these sequences may be used to clone and express the HCLI proteins as described.

As will be appreciated by those having skill in the art, it may be advantageous to produce HCLI polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript polynucleotiderated from the naturally occurring sequence.

The nucleotide sequences of the present invention can be engineered using methods polynucleotidely known in the art in order to alter the HCLI polypeptide-encoding sequences for a variety of reasons, including, but not limited to, alterations which modify the cloning, processing, and/or expression of the polynucleotide product. DNA shuffling by random fragmentation, PCR reassembly of polynucleotide fragments and synthetic oligonucleotides may be used to engineer the nucleotide sequences. For example, site-directed mutapolynucleotidesis may be used to insert new restriction sites, alter glycosylation patterns, change codon preference, produce splice variants, or introduce mutations, and the like.

In another embodiment of the present invention, natural, modified, or recombinant nucleic acid sequences encoding the HCLI polypeptide may be ligated to a heterologous sequence to encode a fusion (or chimeric or hybrid) protein. For example, a fusion protein can comprise all or part of the amino acid sequence as set forth in SEQ ID NO: 2 and an amino acid sequence of an Fc portion (or constant region) of a human immunoglobulin protein. The fusion protein may further comprise an amino acid sequence that differs from SEQ ID NO: 2 only by conservative substitutions. As another example, to screen peptide libraries for inhibitors of HCLI activity, it may be useful to polynucleotiderate a chimeric HCLI protein that can be recognized by a commercially available antibody. A fusion protein may also be engineered to contain a cleavage site located between the HCLI protein-encoding sequence and the heterologous protein sequence, so that the HCLI protein may be cleaved and purified away from the heterologous moiety.

In a further embodiment, sequences encoding the HCLI polypeptide may be synthesized in whole, or in part, using chemical methods well known in the art (see, for example, M. H. Caruthers et al., 1980, Nucl. Acids Res. Symp. Ser., 215-223 and T. Horn et al., 1980, Nucl. Acids Res. Symp. Ser., 225-232). Alternatively, the HCLI protein itself, or a fragment or portion thereof, may be produced using chemical methods to synthesize the amino acid sequence of the HCLI polypeptide, or a fragment or portion thereof. For example, peptide synthesis can be performed using various solid-phase techniques (J. Y. Roberge et al., 1995, Science, 269:202-204) and automated synthesis can be achieved, for example, using the ABI 431A Peptide Synthesizer (PE Biosystems).

The newly synthesized HCLI polypeptide or peptide can be substantially purified by preparative high performance liquid chromatography (e.g., T. Creighton, 1983, Proteins, Structures and Molecular Principles, W. H. Freeman and Co., New York, N.Y.), by reverse-phase high performance liquid chromatography (HPLC), or other purification methods as known and practiced in the art. The composition of the synthetic peptides may be confirmed by amino acid analysis or sequencing (e.g., the Edman degradation procedure; Creighton, supra). In addition, the amino acid sequence of an HCLI polypeptide, or any portion thereof, can be altered during direct synthesis and/or combined using chemical methods with sequences from other proteins, or any part thereof, to produce a variant polypeptide.

To express a biologically active HCLI polypeptide or peptide, the nucleotide sequences encoding the HCLI polypeptide, or functional equivalents, may be inserted into an appropriate expression vector, i.e., a vector, which contains the necessary elements for the transcription and translation of the inserted coding sequence.

In an embodiment of the present invention, an expression vector contains an isolated and purified polynucleotide sequence as set forth in SEQ ID NO: 1 encoding human HCLI, or a functional fragment thereof, in which the human HCLI comprises the amino acid sequence as set forth in SEQ ID NO: 2. Alternatively, an expression vector can contain the complement of the aforementioned HCLI nucleic acid sequence.

Expression vectors derived from retroviruses, adenovirus, herpes or vaccinia viruses, or from various bacterial plasmids can be used for the delivery of nucleotide sequences to a target organ, tissue or cell population. Methods, which are well known to those skilled in the art, may be used to construct expression vectors containing sequences encoding the HCLI polypeptide along with appropriate transcriptional and translational control elements. These methods include in vitro recombinant DNA techniques, synthetic techniques, and in vivo polynucleotide recombination. Such techniques are described in the most recent edition of J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

A variety of expression vector/host systems may be utilized to contain and express sequences encoding the HCLI polypeptide or peptides. Such expression vector/host systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) and tobacco mosaic virus (TMV)), or with bacterial expression vectors (e.g., Ti or pBR322 plasmids); or animal cell systems, including mammalian cell systems. The host cell employed is not limiting to the present invention. Preferably, the host cell of the invention contains an expression vector comprising an isolated and purified polynucleotide having a nucleic acid sequence selected from SEQ ID NO: 1 and encoding the HCLI of this invention, or a functional fragment thereof, comprising an amino acid sequence as set forth in SEQ ID NO: 2.

Bacterial artificial chromosomes (BACs) may be used to deliver larger fragments of DNA than can be contained and expressed in a plasmid vector. BACs are vectors used to clone DNA sequences of 100-300 kb, on average 150 kb, in size in E. coli cells. BACs are constructed and delivered via conventional delivery methods (e.g., liposomes, polycationic amino polymers, or vesicles) for therapeutic purposes.

“Control elements” or “regulatory sequences” are those non-translated regions of the vector, e.g., enhancers, promoters, 5′ and 3′ untranslated regions, which interact with host cellular proteins to carry out transcription and translation. Such elements may vary in their strength and specificity. Depending on the vector system and host utilized, any number of suitable transcription and translation elements, including constitutive and inducible promoters, may be used. Specific initiation signals may also be used to achieve more efficient translation of sequences encoding an HCLI polypeptide. Such signals include the ATG initiation codon and adjacent sequences. In cases where sequences encoding an HCLI polypeptide, its initiation codon, and upstream sequences are inserted into the appropriate expression vector, no additional transcriptional or translational control signals may be needed. However, in cases where only an HCLI coding sequence, or a fragment thereof, is inserted, exogenous translational control signals, including the ATG initiation codon, are optimally provided. Furthermore, the initiation codon should be in the correct reading frame to insure translation of the entire insert. Exogenous translational elements and initiation codons can be of various origins, both natural and synthetic. The efficiency of expression can be enhanced by the inclusion of enhancers which are appropriate for the particular cell system that is used, such as those described in the literature (see, e.g., D. Scharf et al., 1994, Results Probl. Cell Differ., 20:125-162).

In bacterial systems, a number of expression vectors may be selected, depending upon the use intended for the expressed HCLI product. For example, when large quantities of expressed protein are needed for the polynucleotideration of antibodies, vectors that direct high level expression of fusion proteins that can be readily purified may be used. Such vectors include, but are not limited to, the multifunctional E. coli cloning and expression vectors such as BLUESCRIPT (Stratapolynucleotide), in which the sequence encoding the HCLI polypeptide can be ligated into the vector in-frame with sequences for the amino-terminal Met and the subsequent 7 residues of β-galactosidase, so that a hybrid protein is produced; pIN vectors (see, G. Van Heeke and S. M. Schuster, 1989, J. Biol. Chem., 264:5503-5509); and the like. pGEX vectors (Promega, Madison, Wis.) can also be used to express foreign polypeptides as fusion proteins with glutathione S-transferase (GST). In polynucleotide, such fusion proteins are soluble and can be easily purified from lysed cells by adsorption to glutathione-agarose beads followed by elution in the presence of free glutathione. Proteins made in such systems can be designed to include heparin, thrombin, or factor XA protease cleavage sites so that the cloned polypeptide of interest can be released from the GST moiety at will.

In mammalian host cells, a number of viral-based expression systems can be utilized. In cases where an adenovirus is used as an expression vector, sequences encoding the HCLI polypeptide may be ligated into an adenovirus transcription/ translation complex containing the late promoter and tripartite leader sequence. Insertion into a non-essential E1 or E3 region of the viral genome may be used to obtain a viable virus which is capable of expressing an HCLI polypeptide in infected host cells (J. Logan and T. Shenk, 1984, Proc. Natl. Acad. Sci., 81:3655-3659). In addition, transcription enhancers, such as the Rous sarcoma virus (RSV) enhancer, may be used to increase expression in mammalian host cells. Other expression systems can also be used, such as, but not limited to yeast, plant, and insect vectors.

Moreover, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells having specific cellular machinery and characteristic mechanisms for such post-translational activities (e.g., CHO, HeLa, MDCK, HEK293, and W138) are available from the American Type Culture Collection (ATCC), 10801 University Boulevard, Manassas, Va. 20110-2209, and may be chosen to ensure the correct modification and processing of the heterologous protein.

Host cells transformed with vectors containing nucleotide sequences encoding an HCLI protein, or fragments thereof, may be cultured under conditions suitable for the expression and recovery of the protein from cell culture. The protein produced by a recombinant cell may be secreted or contained intracellularly depending on the sequence and/or the vector used. As will be understood by those having skill in the art, expression vectors containing polynucleotides which encode an HCLI protein can be designed to contain signal sequences which direct secretion of the HCLI protein through a prokaryotic or eukaryotic cell membrane. Other constructions can be used to join nucleic acid sequences encoding an HCLI protein to a nucleotide sequence encoding a polypeptide domain, which will facilitate purification of soluble proteins. Such purification facilitating domains include, but are not limited to, metal chelating peptides such as histidine-tryptophan modules that allow purification on immobilized metals; protein A domains that allow purification on immobilized immunoglobulin; and the domain utilized in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the HCLI protein may be used to facilitate purification. One such expression vector provides for expression of a fusion protein containing HCLI and a nucleic acid encoding 6 histidine residues preceding a thioredoxin or an enterokinase cleavage site. The histidine residues facilitate purification on IMAC (immobilized metal ion affinity chromatography) as described by J. Porath et al., 1992, Prot. Exp. Purif., 3:263-281, while the enterokinase cleavage site provides a means for purifying the 6 histidine residue tag from the fusion protein. For a discussion of suitable vectors for fusion protein production, see D. J. Kroll et al., 1993; DNA Cell Biol., 12:441-453.

Any number of selection systems may be used to recover transformed cell lines. These include, but are not limited to, the Herpes Simplex Virus thymidine kinase (HSV TK), (M. Wigler et al., 1977, Cell, 11 :223-32) and adenine phosphoribosyltransferase (I. Lowy et al., 1980, Cell, 22:817-23) polynucleotides which can be employed in tk⁻ or aprt⁻ cells, respectively. Also, anti-metabolite, antibiotic or herbicide resistance can be used as the basis for selection; for example, dhfr, which confers resistance to methotrexate (M. Wigler et al., 1980, Proc. Natl. Acad. Sci., 77:3567-70); npt, which confers resistance to the aminoglycosides neomycin and G-418 (F. Colbere-Garapin et al., 1981, J. Mol. Biol., 150:1-14); and als or pat, which confer resistance to chlorsulfuron and phosphinotricin acetyltransferase, respectively (Murry, supra). Additional selectable polynucleotides have been described, for example, trpB, which allows cells to utilize indole in place of tryptophan, or hisD, which allows cells to utilize histinol in place of histidine (S. C. Hartman and R. C. Mulligan, 1988, Proc. Natl. Acad. Sci., 85:8047-51). Recently, the use of visible markers has gained popularity with such markers as the anthocyanins, β-glucuronidase and its substrate GUS, and luciferase and its substrate luciferin, which are widely used not only to identify transformants, but also to quantify the amount of transient or stable protein expression that is attributable to a specific vector system (C. A. Rhodes et al., 1995, Methods Mol. Biol., 55:121-131).

Although the presence or absence of marker polynucleotide expression suggests that the polynucleotide of interest is also present, the presence and expression of the desired polynucleotide of interest may need to be confirmed. For example, if the nucleic acid sequence encoding the HCLI polypeptide is inserted within a marker polynucleotide sequence, recombinant cells containing a polynucleotide sequence encoding the HCLI polypeptide can be identified by the absence of marker polynucleotide function. Alternatively, a marker polynucleotide can be placed in tandem with a sequence encoding the HCLI polypeptide under the control of a single promoter. Expression of the marker polynucleotide in response to induction or selection typically indicates co-expression of the tandem polynucleotide.

A wide variety of labels and conjugation techniques are known and employed by those skilled in the art and may be used in various nucleic acid and amino acid assays. Means for producing labeled hybridization or PCR probes for detecting sequences related to polynucleotides encoding an HCLI polypeptide include oligo-labeling, nick translation, end-labeling, or PCR amplification using a labeled nucleotide. Alternatively, the sequences encoding an HCLI polypeptide of this invention, or any portion or fragment thereof, can be cloned into a vector for the production of an mRNA probe. Such vectors are known in the art, are commercially available, and may be used to synthesize RNA probes in vitro by addition of an appropriate RNA polymerase, such as T7, T3, or SP(6) and labeled nucleotides. These procedures may be conducted using a variety of commercially available kits (e.g., Amersham Pharmacia Biotech, Promega and U.S. Biochemical Corp.). Suitable reporter molecules or labels which can be used include radionucleotides, enzymes, fluorescent, chemiluminescent, or chromogenic agents, as well as substrates, cofactors, inhibitors, magnetic particles, and the like.

Alternatively, host cells which contain the nucleic acid sequence coding for an HCLI polypeptide of the invention and which express the HCLI polypeptide product may be identified by a variety of procedures known to those having skill in the art. These procedures include, but are not limited to, DNA-DNA or DNA-RNA hybridizations and protein bioassay or immunoassay techniques, including membrane, solution, or chip based technologies, for the detection and/or quantification of nucleic acid or protein.

The presence of polynucleotide sequences encoding HCLI polypeptides can be detected by DNA-DNA or DNA-RNA hybridization, or by amplification using probes, portions, or fragments of polynucleotides encoding an HCLI polypeptide. Nucleic acid amplification based assays involve the use of oligonucleotides or oligomers based on the nucleic acid sequences encoding an HCLI polypeptide to detect transformants containing DNA or RNA encoding an HCLI polypeptide.

In addition to recombinant production, fragments of the HCLI polypeptide may be produced by direct peptide synthesis using solid phase techniques (J. Merrifield, 1963, J. Am. Chem. Soc., 85:2149-2154). Protein synthesis may be performed using manual techniques or by automation. Automated synthesis may be achieved, for example, using ABI 431 A Peptide Synthesizer (PE Biosystems). Various fragments of the HCLI polypeptide can be chemically synthesized separately and then combined using chemical methods to produce the full length molecule.

Diagnostic Assays

In another embodiment of the present invention, antibodies which specifically bind to the HCLI polypeptide may be used for the diagnosis of conditions or diseases characterized by expression (or overexpression) of the HCLI polynucleotide or polypeptide, or in assays to monitor patients being treated with the HCLI polypeptide, or agonists, antagonists, or inhibitors of the novel HCLI. The antibodies useful for diagnostic purposes can be prepared in the same manner as those described herein for use in therapeutic methods. Diagnostic assays for the HCLI polypeptide include methods which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules known to those in the art may be used, several of which are described herein.

Another embodiment of the present invention contemplates a method of detecting an HCLI homologue, or an antibody-reactive fragment thereof, in a sample. The method comprises a) contacting the sample with an antibody specific for an HCLI polypeptide of the present invention, or an antigenic fragment thereof, under conditions in which an antigen-antibody complex can form between the antibody and the polypeptide or antigenic fragment thereof in the sample; and b) detecting the antigen-antibody complex formed in step a), wherein detection of the complex indicates the presence of the HCLI polypeptide, or an antigenic fragment thereof, in the sample.

Several assay protocols including enzyme-linked immunosorbent assay (ELISA), radioimmunoassay (RIA), and fluorescence activated cell sorting (FACS) for measuring an HCLI polypeptide are known in the art and provide a basis for diagnosing altered or abnormal levels of HCLI polypeptide expression. Normal or standard values for HCLI polypeptide expression are established by combining body fluids or cell extracts taken from normal mammalian subjects, preferably human, with antibody to the HCLI polypeptide under conditions suitable for complex formation. The amount of standard complex formation may be quantified by various methods; photometric means are preferred. Quantities of HCLI polypeptide expressed in a subject or test sample, control sample, and disease samples from biopsied tissues are compared with the standard values. Deviation between standard and subject values establishes the parameters for diagnosing disease.

A variety of protocols for detecting and measuring the expression of HCLI polypeptide using either polyclonal or monoclonal antibodies specific for the polypeptide, or epitopic portions thereof, are known and practiced in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive with two non-interfering epitopes on an HCLI polypeptide is preferred, but a competitive binding assay may also be employed. These and other assays are described in the art as represented by the publication of R. Hampton et al., 1990; Serological Methods, a Laboratory Manual, APS Press, St Paul, Minn. and D. E. Maddox et al., 1983; J. Exp. Med., 158:1211-1216).

Another embodiment of the present invention encompasses a method of using an HCLI-encoding polynucleotide sequence to isolate and/or purify a molecule or compound in a sample, wherein the molecule or compound specifically binds to the polynucleotide. The method comprises: a) combining an HCLI-encoding polynucleotide of the invention with a sample undergoing testing to determine if the sample contains the molecule or compound, under conditions to allow specific binding; b) detecting specific binding between the HCLI-encoding polynucleotide and the molecule or compound, if present; c) recovering the bound polynucleotide; and d) separating the polynucleotide from the molecule or compound, thereby obtaining a purified or substantially purified molecule or compound.

This invention also relates to a method of using HCLI polynucleotides as diagnostic reagents. For example, the detection of a mutated form of the HCLI polynucleotide associated with a dysfunction can provide a diagnostic tool that can add to or define diagnosis of a disease or susceptibility to a disease which results from under-expression, over-expression, or altered expression of HCLI. Individuals carrying mutations in the HCLI polynucleotide may be detected at the DNA level by a variety of techniques.

Nucleic acids for diagnosis may be obtained from various sources of a subject, for example, from cells, tissue, blood, urine, saliva, tissue biopsy or autopsy material. Genomic DNA may be used directly for detection or may be amplified by using PCR or other amplification techniques prior to analysis. RNA or cDNA may also be used in similar fashion. Deletions and insertions in an HCLI-encoding polynucleotide can be detected by a change in size of the amplified product compared with that of the normal genotype. Hybridizing amplified DNA to labeled GPCR polynucleotide sequences can identify point mutations. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, for example, Myers et al., Science (1985) 230:1242. Sequence changes at specific locations may also be revealed by nuclease protection assays, such as RNase and S1 protection or the chemical cleavage method. (See Cotton et al., Proc. Natl. Acad. Sci., USA (1985) 85:43297-4401).

In another embodiment, an array of oligonucleotide probes comprising the HCLI nucleotide sequence or fragments thereof can be constructed to conduct efficient screening of, for example, polynucleotide mutations. Array technology methods are well known, have polynucleotide applicability and can be used to address a variety of questions in molecular polynucleotides, including polynucleotide expression, polynucleotide linkage, and polynucleotide variability (see for example: M. Chee et al., Science, 274:610-613, 1996).

Yet another aspect of the present invention involves a method of screening a library of molecules or compounds with an HCLI-encoding polynucleotide to identify at least one molecule or compound therein which specifically binds to the HCLI polynucleotide sequence. Such a method includes a) combining an HCLI-encoding polynucleotide of the present invention with a library of molecules or compounds under conditions to allow specific binding; and b) detecting specific binding, thereby identifying a molecule or compound, which specifically binds to an HCLI-encoding polynucleotide sequence, wherein the library is selected from DNA molecules, RNA molecules, artificial chromosome constructions, PNAs, peptides and proteins.

The present invention provides diagnostic assays for determining or monitoring through detection of a mutation in the HCLI polynucleotide (polynucleotide) described herein susceptibility to the following conditions, diseases, or disorders:

In addition, such diseases, disorder, or conditions, can be diagnosed by methods of determining from a sample derived from a subject having an abnormally decreased or increased level of HCLI polypeptide or HCLI mRNA. Decreased or increased expression can be measured at the RNA level using any of the methods well known in the art for the quantification of polynucleotides, such as, for example, PCR, RT-PCR, RNase protection, Northern blotting and other hybridization methods. Assay techniques that can be used to determine levels of a protein, such as HCLI in a sample derived from a host are well known to those of skill in the art. Such assay methods include, without limitation, radioimmunoassays, competitive-binding assays, Western Blot analysis and ELISA assays.

In another of its aspects, this invention relates to a kit for detecting and diagnosing an HCLI-associated disease or susceptibility to such a disease, which comprises an HCLI or HCLI variant polynucleotide, preferably the nucleotide sequence of SEQ ID NOS: 1, 3, or 16, or a fragment thereof; or a nucleotide sequence complementary to the HCLI polynucleotide of SEQ ID NOS: 1, 3, or 16; or an HCLI or HCLI variant polypeptide, preferably the polypeptide of SEQ ID NOS: 2, 4, or 17, or a fragment thereof; or an antibody to the HCLI or HCLI variant polypeptide, preferably to the polypeptide of SEQ ID NOS: 2, 4, or 17, an epitope-containing portion thereof, or combinations of the foregoing. It will be appreciated that in any such kit, any of the previously mentioned components may comprise a substantial component. Also preferably included are instructions for use.

The HCLI polynucleotides which may be used in the diagnostic assays according to the present invention include oligonucleotide sequences, complementary RNA and DNA molecules, and PNAs. The polynucleotides may be used to detect and quantify HCLI-encoding nucleic acid expression in biopsied tissues in which expression (or under- or over-expression) of the HCLI polynucleotide may be determined, as well as correlated with disease. The diagnostic assays may be used to distinguish between the absence of HCLI, the presence of HCLI, or the excess expression of HCLI, and to monitor the regulation of HCLI polynucleotide levels during therapeutic treatment or intervention.

In a related aspect, hybridization with PCR probes which are capable of detecting polynucleotide sequences, including genomic sequences, encoding an HCLI polypeptide according to the present invention, or closely related molecules, may be used to identify nucleic acid sequences which encode an HCLI polypeptide. The specificity of the probe, whether it is made from a highly specific region, for example, about 8 to 10 contiguous nucleotides in the 5′ regulatory region, or a less specific region, for example, especially in the 3′ coding region, and the stringency of the hybridization or amplification (maximal, high, intermediate, or low) will determine whether the probe identifies only naturally occurring sequences encoding HCLI polypeptide, alleles thereof, or related sequences, as understood by the skilled practitioner.

Probes may also be used for the detection of related sequences, and should preferably contain at least 50% of the nucleotides encoding the HCLI polypeptide. The hybridization probes or primers of this invention may be DNA or RNA and may be derived from the nucleotide sequences of SEQ ID NO: 1, or may be derived from genomic sequence, including promoter, enhancer elements, and introns of the naturally occurring HCLI protein, wherein the probes or primers comprise a polynucleotide sequence capable of hybridizing with a polynucleotide of SEQ ID NO: 1, under low, moderate, or high stringency conditions.

Methods for producing specific hybridization probes for DNA encoding the HCLI polypeptide include the cloning of a nucleic acid sequence that encodes the HCLI polypeptide, or HCLI derivatives, into vectors for the production of mRNA probes. Such vectors are known in the art, or are commercially available, and may be used to synthesize RNA probes in vitro by means of the addition of the appropriate RNA polymerases and the appropriate labeled nucleotides. Hybridization probes may be labeled by a variety of detector/reporter groups, including, but not limited to, radionuclides such as ³²P or ³⁵S, or enzymatic labels, such as alkaline phosphatase coupled to the probe via avidin/biotin coupling systems, and the like.

The polynucleotide sequence encoding the HCLI polypeptide of this invention, or fragments thereof, may be used for the diagnosis of disorders associated with expression of HCLI. The polynucleotide sequence encoding the HCLI polypeptide may be used in Southern or Northern analysis, dot blot, or other membrane-based technologies; in PCR technologies; or in dipstick, pin, ELISA or chip assays utilizing fluids or tissues from patient biopsies to detect the status of, for example, levels of, or overexpression of, HCLI, or to detect altered HCLI expression or levels. Such qualitative or quantitative methods are commonly practiced in the art.

In a particular aspect, a nucleotide sequence encoding HCLI polypeptide as described herein may be useful in assays that detect activation or induction of various neoplasms, cancers, or other HCLI-related diseases, disorders, or conditions. The nucleotide sequence encoding an HCLI polypeptide may be labeled by standard methods, and added to a fluid or tissue sample from a patient, under conditions suitable for the formation of hybridization complexes. After a suitable incubation period, the sample is washed and the signal is quantified and compared with a standard value. If the amount of signal in the biopsied or extracted sample is significantly altered from that of a comparable control sample, the nucleotide sequence has hybridized with nucleotide sequence present in the sample, and the presence of altered levels of nucleotide sequence encoding the HCLI polypeptide in the sample indicates the presence of the associated disease. Such assays may also be used to evaluate the efficacy of a particular therapeutic treatment regimen in animal studies, in clinical trials, or in monitoring the treatment or responsiveness of an individual patient.

Once disease is established and a treatment protocol is initiated, hybridization assays may be repeated on a regular basis to evaluate whether the level of expression in the patient begins to approximate that which is observed in a normal individual. The results obtained from successive assays may be used to show the efficacy of treatment over a period ranging from several days to months.

With respect to ion channel related disorders, the presence of an abnormal amount or level of an HCLI transcript in biopsied tissue from an individual may indicate a predisposition for the development of the disease, or may provide a means for detecting the disease prior to the appearance of actual clinical symptoms. A more definitive diagnosis of this type may allow health practitioners to employ preventative measures or aggressive treatment earlier, thereby preventing the development or further progression of the disorder.

Additional diagnostic uses for oligonucleotides designed from the nucleic acid sequences encoding the novel HCLI polypeptide of this invention can involve the use of PCR. Such oligomers may be chemically synthesized, polynucleotiderated enzymatically, or produced from a recombinant source. Oligomers will preferably comprise two nucleotide sequences: one with sense orientation (5′→3′) and another with antisense orientation (3′→5′), employed under optimized conditions for identification of a specific polynucleotide or condition. The same two oligomers, nested sets of oligomers, or even a depolynucleotiderate pool of oligomers may be employed under less stringent conditions for detection and/or quantification of closely related DNA or RNA sequences.

Methods suitable for quantifying the expression of HCLI include radiolabeling or biotinylating nucleotides, co-amplification of a control nucleic acid, and standard curves onto which the experimental results are interpolated (P. C. Melby et al., 1993, J. Immunol. Methods, 159:235-244; and C. Duplaa et al., 1993, Anal. Biochem., 229-236). The speed of quantifying multiple samples may be accelerated by running the assay in an ELISA format where the oligomer of interest is presented in various dilutions and a spectrophotometric or colorimetric response gives rapid quantification.

In another embodiment of the invention, a compound to be tested can be radioactively, colorimetrically or fluorimetrically labeled using methods well known in the art and incubated with the HCLI polypeptide for testing. After incubation, it is determined whether the test compound is bound to the HCLI polypeptide. If so, the compound is to be considered a potential agonist or antagonist. Functional assays are performed to determine whether the HCLI channel function is activated (or enhanced or increased) or inhibited (or decreased or reduced) with relation to test compounds. These assays include, but are not limited to, ion fluxes, ion transport, HCLI cellular localization (including translocation to the plasma membrane), membrane potential regulation, voltage-dependent chloride gating, electrical excitability assays (for example, voltage-clamp asssays), pH changes, pH regulation, and acid and water secretion. These types of responses can either be present in the host cell or introduced into the host cell along with HCLI.

The present invention further embraces a method of screening for candidate compounds capable of modulating the activity of an HCLI-encoding polynucleotide. Such a method comprises a) contacting a test compound with a cell, cell membrane or tissue expressing an HCLI polypeptide of the invention (e.g., recombinant expression); and b) selecting as candidate modulating compounds those test compounds that modulate activity of the HCLI polypeptide. Those candidate compounds which modulate HCLI activity are preferably agonists or antagonists, more preferably antagonists of HCLI activity.

The cloning and sequencing of the HCLI plynucleotide provides the ability to polynucleotiderate recombinant host cells useful in expressing all or a portion of the HCLI protein allowing for screening of natural products and synthetic compounds that bind to and/or modulate HCLI protein activity. A process for detecting HCLI protein modulators requires transforming a suitable vector into compatible host cells as described within. Transformed cells are then treated with test substances (e.g., synthetic compounds or natural products), and channel activity is measured and/or assessed in the presence and absence of the test substance.

Therapeutic Assays

The HCLI protein according to this invention may play a role in motility by setting the membrane potential, and therefore determining excitability, in smooth muscle cells, interstitial cells of Cajal, enteric neurons located within the GI tract and leukocytes, such as resident macrophage cells. As the HCLI protein is a chloride channel protein, HCLI may also play a role in epithelial transport processes including acid secretion and water secretion. In particular, as HCLI is highly expressed in stomach, lung and heart, HCLI may play a role in chloride ion channel-related functions in these tissues.

In yet another embodiment of the present invention, an antagonist or inhibitory agent of the HCLI polypeptide may be administered therapeutically to an individual to prevent or treat a chloride channel related-disorder. Such disorders may include, but are not limited to, Myotonia congenita, retinal depolynucleotideration, male infertility, neurodepolynucleotideration, Dent's disease, X-linked nephrolithiasis syndromes, infantile malignant osteopetrosis, nephrogenic diabetes insipidus, and Bartter's syndrome.

A preferred method of treating an HCLI associated disease, disorder, syndrome, or condition in a mammal comprises administration of a modulator, preferably an inhibitor or antagonist, of an HCLI polypeptide or homologue of the invention, in an amount effective to treat, reduce, and/or ameliorate the symptoms incurred by the HCLI-associated disease, disorder, syndrome, or condition. In some instances, an agonist or enhancer of an HCLI polypeptide or homologue of the invention is administered in an amount effective to treat and/or ameliorate the symptoms incurred by an HCLI-related disease, disorder, syndrome, or condition. In other instances, the administration of a novel HCLI polypeptide or homologue thereof pursuant to the present invention is envisioned for administration to treat an HCLI associated disease.

In yet another embodiment of the present invention, an expression vector containing the complement of the polynucleotide encoding an HCLI polypeptide is administered to an individual to treat or prevent any one of the types of diseases, disorders, or conditions previously described, in an antisense therapy method.

The HCLI protein, modulators, including antagonists, antibodies, and agonists, complementary sequences, or vectors of the present invention can also be administered in combination with other appropriate therapeutic agents as necessary or desired. Selection of the appropriate agents for use in combination therapy may be made by the skilled practitioner in the art, according to conventional pharmaceutical and clinical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects or adverse events.

Antagonists or inhibitors of the HCLI polypeptide of this invention can be produced using methods which are polynucleotidely known in the art. In particular, purified HCLI protein, or fragments thereof, can be used to produce antibodies, or to screen libraries of pharmaceutical agents, to identify those which specifically bind to the novel HCLI polypeptide as described herein.

Antibodies specific for HCLI polypeptide, or immunogenic peptide fragments thereof, can be polynucleotiderated using methods that have long been known and conventionally practiced in the art. Such antibodies may include, but are not limited to, polyclonal, monoclonal, neutralizing antibodies, (i.e., those which inhibit dimer formation), chimeric, single chain, Fab fragments, and fragments produced by an Fab expression library. A non-limiting example of the HCLI polypeptide or immunogenic fragments thereof that may be used to polynucleotiderate antibodies is provided in SEQ ID NO: 2.

For the production of antibodies, various hosts, including goats, rabbits, sheep, rats, mice, humans, and others, can be immunized by injection with the HCLI polypeptide, or any immunogenic and/or epitope-containing fragment or oligopeptide thereof, which have immunogenic properties. Depending on the host species, various adjuvants may be used to increase the immunological response. Non-limiting examples of suitable adjuvants include Freund's (incomplete), mineral gels such as aluminum hydroxide or silica, and surface active substances such as lysolecithin, pluronic polyols, polyanions, peptides, oil emulsions, KLH, and dinitrophenol. Adjuvants typically used in humans include BCG (bacilli Calmette Guérin) and Corynebacterium parvumn.

Preferably, the HCLI polypeptide, peptides, fragments, or oligopeptides used to induce antibodies to the HCLI polypeptide immunogens have an amino acid sequence of at least five amino acids in length, and more preferably, at least 7-10, or more, amino acids. It is also preferable that the immunogens are identical to a portion of the amino acid sequence of the natural protein; they may also contain the entire amino acid sequence of a small, naturally occurring molecule. The peptides, fragments or oligopeptides may comprise a single epitope or antigenic determinant or multiple epitopes. Short stretches of HCLI amino acids may be fused with another protein as carrier, such as KLH, such that antibodies are produced against the chimeric molecule.

Monoclonal antibodies to the HCLI polypeptide, or immunogenic fragments thereof, may be prepared using any technique which provides for the production of antibody molecules by continuous cell lines in culture. Such techniques are conventionally used in the art. These include, but are not limited to, the hybridoma technique, the human B-cell hybridoma technique, and the EBV-hybridoma technique (G. Kohler et al., 1975, Nature, 256:495-497; D. Kozbor et al., 1985, J. Immunol. Methods, 81:31-42; R. J. Cote et al.,.1983, Proc. Natl. Acad. Sci. USA, 80:2026-2030; and S. P. Cole et al., 1984, Mol. Cell Biol., 62:109-120). The production of monoclonal antibodies to immunogenic proteins and peptides is well known and routinely used in the art.

In addition, techniques developed for the production of “chimeric antibodies,” the splicing of mouse antibody polynucleotides to human antibody polynucleotides to obtain a molecule with appropriate antigen specificity and biological activity can be used (S. L. Morrison et al., 1984, Proc. Natl. Acad. Sci. USA, 81:6851-6855; M. S. Neuberger et al., 1984, Nature, 312:604-608; and S. Takeda et al., 1985, Nature, 314:452-454). Alternatively, techniques described for the production of single chain antibodies may be adapted, using methods known in the art, to produce HCLI polypeptide-specific single chain antibodies. Antibodies with related specificity, but of distinct idiotypic composition, may be polynucleotiderated by chain shuffling from random combinatorial immunoglobulin libraries (D. R. Burton, 1991, Proc. Natl. Acad. Sci. USA, 88:11120-3). Antibodies may also be produced by inducing in vivo production in the lymphocyte population or by screening recombinant immunoglobulin libraries or panels of highly specific binding reagents as disclosed in the literature (R. Orlandi et al., 1989, Proc. Natl. Acad. Sci. USA, 86:3833-3837 and G. Winter et al., 1991, Nature, 349:293-299).

Antibody fragments, which contain specific binding sites for an HCLI polypeptide, may also be polynucleotiderated. For example, such fragments include, but are not limited to, F(ab′)₂ fragments which can be produced by pepsin digestion of the antibody molecule and Fab fragments which can be polynucleotiderated by reducing the disulfide bridges of the F(ab′)₂ fragments. Alternatively, Fab expression libraries may be constructed to allow rapid and easy identification of monoclonal Fab fragments with the desired specificity (e.g., W. D. Huse et al., 1989, Science, 254.1275-1281).

Various immunoassays can be used for screening to identify antibodies having the desired specificity. Numerous protocols for competitive binding or immunoradiometric assays using either polyclonal or monoclonal antibodies with established specificities are well known in the art. Such immunoassays typically involve measuring the formation of complexes between an HCLI polypeptide and its specific antibody. A two-site, monoclonal-based immunoassay utilizing monoclonal antibodies reactive with two non-interfering HCLI polypeptide epitopes is suitable, but a competitive binding assay may also be employed (Maddox, supra).

To induce an immunological response in a mammal, a host animal is inoculated with an HCLI polypeptide, or a fragment thereof, of this invention in an amount adequate to produce an antibody and/or a T cell immune response to protect the animal from a disease or disorder associated with the expression or production of an HCLI polypeptide. Yet another aspect of the invention relates to a method of inducing immunological response in a mammal, if applicable or required. Such a method comprises delivering HCLI polypeptide via a vector directing expression of HCLI polynucleotide in vivo in order to induce such an immunological response to produce antibody to protect said animal from HCLI-related diseases.

A further aspect of the invention relates to an immunological vaccine or immunogen formulation or composition which, when introduced into a mammalian host, induces an immunological response in that mammal to an HCLI polypeptide wherein the composition comprises an HCLI polypeptide or HCLI polynucleotide. The vaccine or immunogen formulation may further comprise a suitable carrier. Since the HCLI polypeptide may be broken down in the stomach, it is preferably administered parenterally (including subcutaneous, intramuscular, intravenous, intradermal, etc., injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents.

The formulations may be presented in unit-dose or multi-dose containers, for example, sealed ampoules and vials, and may be stored in a freeze-dried condition requiring only the addition of the sterile liquid carrier immediately prior to use. A vaccine formulation may also include adjuvant systems for enhancing the immunogenicity of the formulation, such as oil-in-water systems and other systems known in the art. The dosage will depend on the specific activity of the vaccine and can be readily determined by routine experimentation.

In an aspect of the present invention, the polynucleotide encoding an HCLI polypeptide, or any fragment or complement thereof, as described herein may be used for therapeutic purposes. For instance, antisense to an HCLI polynucleotide encoding an HCLI polypeptide, may be used in situations in which it would be desirable to block the transcription of HCLI mRNA. In particular, cells may be transformed, transfected, or injected with sequences complementary to polynucleotides encoding HCLI polypeptide. Thus, complementary molecules may be used to modulate HCLI polynucleotide and polypeptide activity, or to achieve regulation of polynucleotide function. Such technology is well known in the art, and sense or antisense oligomers or oligonucleotides, or larger fragments, can be designed from various locations along the coding or control regions of the HCLI polynucleotide sequence encoding the novel HCLI polypeptide.

Polypeptides used in treatment can also be polynucleotiderated endogenously in the subject, in treatment modalities often referred to as “polynucleotide therapy”. Thus for example, cells from a subject may be engineered with a polynucleotide, such as DNA or RNA, to encode a polypeptide ex vivo, for example, by the use of a retroviral plasmid vector. The cells can then be introduced into the subject's body in which the desired polypeptide is expressed.

A polynucleotide encoding an HCLI polypeptide can be turned off by transforming a cell or tissue with an expression vector that expresses high levels of an HCLI polypeptide-encoding polynucleotide, or a fragment thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and even longer if appropriate replication elements are designed to be part of the vector system.

Modifications of polynucleotide expression can be obtained by designing antisense molecules or complementary nucleic acid sequences (DNA, RNA, or PNA), to the control, 5′, or regulatory regions of an HCLI polynucleotide sequence encoding an HCLI polypeptide, (e.g., a signal sequence, promoters, enhancers, and introns). Oligonucleotides may be derived from the transcription initiation site, for example, between positions −10 and +10 from the start site.

Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described (see, for example, J. E. Gee et al., 1994, In: B. E. Huber and B. I. Carr, Molecular and Immunologic Approaches, Futura Publishing Co., Mt. Kisco, N.Y.). The antisense molecule or complementary sequence may also be designed to block translation of mRNA by preventing the transcript from binding to ribosomes.

Many methods for introducing vectors into cells or tissues are available and are equally suitable for use in vivo, in vitro, and ex vivo. For ex vivo therapy, vectors may be introduced into stem cells or bone marrow cells obtained from the patient and clonally propagated for autologous transplant back into that same patient. Delivery by transfection, direct injection (e.g., microparticle bombardment) and by liposome injections may be achieved using methods which are well known in the art.

Any of the therapeutic methods described above can be applied to any individual in need of such therapy, including, for example, mammals such as dogs, cats, cows, horses, rabbits, monkeys, and most preferably, humans.

Polypeptide or polynucleotides and/or agonist or antagonists of the present invention may also be used to increase the efficacy of a pharmaceutical composition, either directly or indirectly. Such a use may be administered in simultaneous conjunction with said pharmaceutical, or separately through either the same or different route of administration (e.g., intravenous for the polynucleotide or polypeptide of the present invention, and orally for the pharmaceutical, among others described herein.).

Polypeptide or polynucleotides and/or agonist or antagonists of the present invention may also be used to prepare individuals for extraterrestrial travel, low gravity environments, prolonged exposure to extraterrestrial radiation levels, low oxygen levels, reduction of metabolic activity, exposure to extraterrestrial pathogens, etc. Such a use may be administered either prior to an extraterrestrial event, during an extraterrestrial event, or both. Moreover, such a use may result in a number of beneficial changes in the recipient, such as, for example, any one of the following, non-limiting, effects: an increased level of hematopoietic cells, particularly red blood cells which would aid the recipient in coping with low oxygen levels; an increased level of B-cells, T-cells, antigen presenting cells, and/or macrophages, which would aid the recipient in coping with exposure to extraterrestrial pathogens, for example; a temporary (i.e., reversible) inhibition of hematopoietic cell production which would aid the recipient in coping with exposure to extraterrestrial radiation levels; increase and/or stability of bone mass which would aid the recipient in coping with low gravity environments; and/or decreased metabolism which would effectively facilitate the recipients ability to prolong their extraterrestrial travel by any one of the following, non-limiting means: (i) aid the recipient by decreasing their basal daily energy requirements; (ii) effectively lower the level of oxidative and/or metabolic stress in recipient (i.e., to enable recipient to cope with increased extraterrestial radiation levels by decreasing the level of internal oxidative/metabolic damage acquired during normal basal energy requirements; and/or (iii) enabling recipient to subsist at a lower metabolic temperature (i.e., cryogenic, and/or sub-cryogenic environment).

Pharmaceutical Preparations

A further embodiment of the present invention embraces pharmaceutical compositions and the administration thereof, in conjunction with a pharmaceutically acceptable carrier, diluent, or excipient, to achieve any of the above-described therapeutic uses and effects. Depending upon the disease treatment, such pharmaceutical compositions can comprise HCLI nucleic acid, polypeptide, or peptides, antibodies to HCLI polypeptide, mimetics, HCLI modulators, such as agonists, antagonists, or inhibitors of an HCLI polypeptide or polynucleotide. The compositions can comprise the active agent or ingredient alone, or in combination with at least one other agent or reagent, such as a stabilizing compound, which may be administered in any sterile, biocompatible pharmaceutical carrier, including, but not limited to, saline, buffered saline, dextrose, and water. The compositions may be administered to a patient alone, or in combination with other agents, drugs, hormones, or biological response modifiers.

The pharmaceutical compositions for use in the present invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, vaginal, or rectal means.

In addition to the active ingredients (e.g., HCLI nucleic acid or polypeptide, or functional fragments thereof, or an HCLI agonist or antagonist), the pharmaceutical compositions may contain pharmaceutically acceptable/physiologically suitable carriers or excipients comprising auxiliaries which facilitate processing of the active compounds into preparations that can be used pharmaceutically. Further details on techniques for formulation and administration are provided in the latest edition of Remington's Pharmaceutical Sciences (Mack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

In addition, pharmaceutical preparations for oral use can be obtained by the combination of active compounds with a solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents may be added, such as cross-linked polyvinyl pyrrolidone, agar, alginic acid, or a physiologically acceptable salt thereof, such as sodium alginate.

Dragee cores may be used in conjunction with physiologically suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification, or to characterize the quantity of active compound, i.e., dosage.

Pharmaceutical preparations, which can be used orally, further include push-fit capsules made of gelatin, as well as soft, scaled capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances, which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions.

For topical or nasal administration, penetrants or permeation agents (enhancers) that are appropriate to the particular barrier to be permeated are used in the formulation. Such penetrants are polynucleotidely known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes.

A pharmaceutical composition can be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, combined with a buffer prior to use. After the pharmaceutical compositions have been prepared, they can be placed in an appropriate container and labeled for treatment of an indicated condition. For administration of an HCLI product, such labeling would include amount, frequency, and method of administration.

Pharmaceutical compositions suitable for use in the present invention include compositions wherein the active ingredients are contained in an effective amount to achieve the intended purpose. The determination of an effective dose or amount is well within the capability of those skilled in the art. For any compound, the therapeutically effective dose can be estimated initially either in cell culture assays, for example, using neoplastic cells, or in animal models, usually mice, rabbits, dogs, or pigs. The animal model may also be used to determine the appropriate concentration range and route of administration. Such information can then be used and extrapolated to determine useful doses and routes for administration in humans.

A therapeutically effective dose refers to that amount of active ingredient, for example, HCLI polynucleotide, HCLI polypeptide, or fragments thereof, antibodies to HCLI polypeptide, agonists, antagonists or inhibitors of HCLI polypeptide, which ameliorates, reduces, diminishes, or eliminates the symptoms or condition. Therapeutic efficacy and toxicity can be determined by standard pharmaceutical procedures in cell cultures or in experimental animals, e.g., ED₅₀ (the dose therapeutically effective in 50% of the population) and LD₅₀ (the dose lethal to 50% of the population). The dose ratio of toxic to therapeutic effects is the therapeutic index, which can be expressed as the ratio, LD₅₀/ED₅₀. Pharmaceutical compositions which exhibit large therapeutic indices are preferred. The data obtained from cell culture assays and animal studies are used in determining a range of dosages for human use. Preferred dosage contained in a pharmaceutical composition is within a range of circulating concentrations that include the ED₅₀ with little or no toxicity. The dosage varies within this range depending upon the dosage form employed, the sensitivity of the patient, and the route of administration.

The practitioner, who will consider the factors related to an individual requiring treatment, will determine the exact dosage. Dosage and administration are adjusted to provide sufficient levels of the active component, or to maintain the desired effect. Factors which may be taken into account include the severity of the individual's disease state; the polynucleotide health of the patient; the age, weight, and gender of the patient; diet; time and frequency of administration; drug combination(s); reaction sensitivities; and tolerance/response to therapy. As a polynucleotide guide, long-acting pharmaceutical compositions may be administered every 3 to 4 days, every week, or once every two weeks, depending on half-life and clearance rate of the particular formulation. Variations in these dosage levels can be adjusted using standard empirical routines for optimization, as is well understood in the art.

As a guide, normal dosage amounts may vary from 0.1 to 100,000 micrograms (μg), up to a total dose of about 1 gram (g), depending upon the route of administration. Guidance as to particular dosages and methods of delivery is provided in the literature and is polynucleotidely available to practitioners in the art. Those skilled in the art will employ different formulations for nucleotides than for proteins or their inhibitors or activators. Similarly, the delivery of polynucleotides or polypeptides will be specific to particular cells, conditions, locations, and the like.

Antibodies

Further polypeptides of the invention relate to antibodies and T-cell antigen receptors (TCR) which immunospecifically bind a polypeptide, polypeptide fragment, or variant of SEQ ID NO: 2, 4, or 17, and/or an epitope, of the present invention (as determined by immunoassays well known in the art for assaying specific antibody-antigen binding). Antibodies of the invention include, but are not limited to, polyclonal, monoclonal, monovalent, bispecific, heteroconjugate, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule. Moreover, the term “antibody” (Ab) or “monoclonal antibody” (Mab) is meant to include intact molecules, as well as, antibody fragments (such as, for example, Fab and F(ab′)2 fragments) which are capable of specifically binding to protein. Fab and F(ab′)2 fragments lack the Fc fragment of intact antibody, clear more rapidly from the circulation of the animal or plant, and may have less non-specific tissue binding than an intact antibody (Wahl et al., J. Nucl. Med. 24:316-325 (1983)). Thus, these fragments are preferred, as well as the products of a FAB or other immunoglobulin expression library. Moreover, antibodies of the present invention include chimeric, single chain, and humanized antibodies.

Most preferably the antibodies are human antigen-binding antibody fragments of the present invention and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, by size in contiguous amino acid residues, or listed in the Figures. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homologue of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologues of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinity to a polypeptide of the invention. Preferred binding affinities include those with a dissociation constant or Kd less than 5×10-2 M, 10-2 M, 5×10-3 M, 10-3 M, 5×10-4 M, 10-4 M, 5×10-5 M, 10-5 M, 5×10-6 M, 10-6M, 5×10-7 M, 107 M, 5×10-8 M, 10-8 M, 5×10-9 M, 10-9 M, 5×10-10 M, 10-10 M, 5×10-11 M, 10-11 M, 5×10-12M, 10-12M, 5×10-13 M, 10-13 M, 5×10-14 M, 10-14 M, 5×10-15 M, or 10-15 M.

The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85 %, at least 80%, at least 75%, at least 70%, at least 60%, or at least 50%.

Antibodies of the present invention may act as agonists or antagonists of the polypeptides of the present invention. For example, the present invention includes antibodies which disrupt the receptor/ligand interactions with the polypeptides of the invention either partially or fully. Preferably, antibodies of the present invention bind an antigenic epitope disclosed herein, or a portion thereof. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or its substrate by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex, and, preferably, do not specifically recognize the unbound receptor or the unbound ligand. Likewise, included in the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides of the invention disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. 111(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996) (which are all incorporated by reference herein in their entireties).

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988) (incorporated by reference herein in its entirety).

As discussed in more detail below, the antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionucleotides, or toxins. See, e.g., PCT publications WO 92/08495; WO 91/14438; WO 89/12624; U.S. Pat. No. 5,314,995; and EP 396,387.

The antibodies of the invention include derivatives that are modified, i.e., by the covalent attachment of any type of molecule to the antibody such that covalent attachment does not prevent the antibody from polynucleotiderating an anti-idiotypic response. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be polynucleotiderated by any suitable method known in the art.

The antibodies of the present invention may comprise polyclonal antibodies. Methods of preparing polyclonal antibodies are known to the skilled artisan (Harlow, et al., Antibodies: A Laboratory Manual, (Cold spring Harbor Laboratory Press, 2^{nd} ed. (1988); and Current Protocols, Chapter 2; which are hereby incorporated herein by reference in its entirety). In a preferred method, a preparation of the HCLI protein is prepared and purified to render it substantially free of natural contaminants. Such a preparation is then introduced into an animal in order to produce polyclonal antisera of greater specific activity. For example, a polypeptide of the invention can be administered to various host animals including, but not limited to, rabbits, mice, rats, etc. to induce the production of sera containing polyclonal antibodies specific for the antigen. The administration of the polypeptides of the present invention may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art. For the purposes of the invention, “immunizing agent” may be defined as a polypeptide of the invention, including fragments, variants, and/or derivatives thereof, in addition to fusions with heterologous polypeptides and other forms of the polypeptides described herein.

Typically, the immunizing agent and/or adjuvant will be injected in the mammal by multiple subcutaneous or intraperitoneal injections, though they may also be given intramuscularly, and/or through IV). The immunizing agent may include polypeptides of the present invention or a fusion protein or variants thereof. Depending upon the nature of the polypeptides (i.e., percent hydrophobicity, percent hydrophilicity, stability, net charge, isoelectric point etc.), it may be useful to conjugate the immunizing agent to a protein known to be immunogenic in the mammal being immunized. Such conjugation includes either chemical conjugation by derivitizing active chemical functional groups to both the polypeptide of the present invention and the immunogenic protein such that a covalent bond is formed, or through fusion-protein based methodology, or other methods known to the skilled artisan. Examples of such immunogenic proteins include, but are not limited to keyhole limpet hemocyanin, serum albumin, bovine thyroglobulin, and soybean trypsin inhibitor. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Additional examples of adjuvants which may be employed includes the MPL-TDM adjuvant (monophosphoryl lipid A, synthetic trehalose dicorynomycolate). The immunization protocol may be selected by one skilled in the art without undue experimentation.

The antibodies of the present invention may comprise monoclonal antibodies. Monoclonal antibodies may be prepared using hybridoma methods, such as those described by Kohler and Milstein, Nature, 256:495 (1975) and U.S. Pat. No. 4,376,110, by Harlow, et al., Antibodies: A Laboratory Manual, (Cold spring Harbor Laboratory Press, 2^{nd ed. (}1988), by Hammerling, et al., Monoclonal Antibodies and T-Cell Hybridomas (Elsevier, N.Y., pp. 563-681 (1981); Köhler et al., Eur. J. Immunol. 6:511 (1976); Köhler et al., Eur. J. Immunol. 6:292 (1976), or other methods known to the artisan. Other examples of methods which may be employed for producing monoclonal antibodies includes, but are not limited to, the human B-cell hybridoma technique (Kosbor et al., 1983, Immunology Today 4:72; Cole et al., 1983, Proc. Nat]. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In a hybridoma method, a mouse, a humanized mouse, a mouse with a human immune system, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include polypeptides of the present invention or a fusion protein thereof. Preferably, the immunizing agent consists of an HCLI polypeptide or, more preferably, with a HCLI polypeptide-expressing cell. Such cells may be cultured in any suitable tissue culture medium; however, it is preferable to culture cells in Earle's modified Eagle's medium supplemented with 10% fetal bovine serum (inactivated at about 56 degrees C.), and supplemented with about 10 g/l of nonessential amino acids, about 1,000 U/ml of penicillin, and about 100 ug/ml of streptomycin. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986), pp. 59-103). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. More preferred are the parent myeloma cell line (SP20) as provided by the ATCC. As inferred throughout the specification, human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the polypeptides of the present invention. Preferably, the binding specificity of monoclonal antibodies produced by the hybridoma cells is determined by immunoprecipitation or by an in vitro binding assay, such as radioimmunoassay (RIA) or enzyme-linked immunoabsorbant assay (ELISA). Such techniques are known in the art and within the skill of the artisan. The binding affinity of the monoclonal antibody can, for example, be determined by the Scatchard analysis of Munson and Pollart, Anal. Biochem., 107:220 (1980).

After the desired hybridoma cells are identified, the clones may be subcloned by limiting dilution procedures and grown by standard methods (Goding, supra, and/or according to Wands et al. (Gastroenterology 80:225-232 (1981)). Suitable culture media for this purpose include, for example, Dulbecco's Modified Eagle's Medium and RPMI-1640. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-sepharose, hydroxyapatite chromatography, gel exclusion chromatography, gel electrophoresis, dialysis, or affinity chromatography.

The skilled artisan would acknowledge that a variety of methods exist in the art for the production of monoclonal antibodies and thus, the invention is not limited to their sole production in hydridomas. For example, the monoclonal antibodies may be made by recombinant DNA methods, such as those described in U.S. Pat. No. 4,816,567. In this context, the term “monoclonal antibody” refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to polynucleotides encoding the heavy and light chains of murine antibodies, or such chains from human, humanized, or other sources). The hydridoma cells of the invention serve as a preferred source of such DNA. Once isolated, the DNA may be placed into expression vectors, which are then transformed into host cells such as Simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567; Morrison et al, supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated polynucleotidely at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art and are discussed in detail in the Examples described herein. In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which polynucleotidely contains high levels of antibodies, can be polynucleotiderated by immunizing mice with positive hybridoma clones.

Accordingly, the present invention provides methods of polynucleotiderating monoclonal antibodies as well as antibodies produced by the method comprising culturing a hybridoma cell secreting an antibody of the invention wherein, preferably, the hybridoma is polynucleotiderated by fusing splenocytes isolated from a mouse immunized with an antigen of the invention with myeloma cells and then screening the hybridomas resulting from the fusion for hybridoma clones that secrete an antibody able to bind a polypeptide of the invention.

Antibody fragments which recognize specific epitopes may be polynucleotiderated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

For example, the antibodies of the present invention can also be polynucleotiderated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage polynucleotide III or polynucleotide VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to polynucleotiderate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties). Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988).

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; Cabilly et al., Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985); U.S. Pat. Nos. 5,807,715; 4,816,567; and 4,816397, which are incorporated herein by reference in their entirety. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). Generally, a humanized antibody has one or more amino acid residues introduced into it from a source that is non-human. These non-human amino acid residues are often referred to as “import” residues, which are typically taken from an “import” variable domain. Humanization can be essentially performed following the methods of Winter and co-workers (Jones et al., Nature, 321:522-525 (1986); Reichmann et al., Nature, 332:323-327 (1988); Verhoeyen et al., Science, 239:1534-1536 (1988), by substituting rodent CDRs or CDR sequences for the corresponding sequences of a human antibody. Accordingly, such “humanized” antibodies are chimeric antibodies (U.S. Pat. No. 4, 816, 567), wherein substantially less than an intact human variable domain has been substituted by the corresponding sequence from a non-human species. In practice, humanized antibodies are typically human antibodies in which some CDR residues and possible some FR residues are substituted from analogous sites in rodent antibodies.

In polynucleotide, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature 332:323-329 (1988)1 and Presta, Curr. Op. Struct. Biol., 2:593-596 (1992).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety. The techniques of cole et al., and Boerder et al., are also available for the preparation of human monoclonal antibodies (cole et al., Monoclonal Antibodies and Cancer Therapy, Alan R. Riss, (1985); and Boerner et al., J. Immunol., 147(1):86-95, (1991)).

Human antibodies can also be produced using transgenic mice which are incapable of expressing functional endogenous immunoglobulins, but which can express human immunoglobulin polynucleotides. For example, the human heavy and light chain immunoglobulin polynucleotide complexes may be introduced randomly or by homologous recombination into mouse embryonic stem cells. Alternatively, the human variable region, constant region, and diversity region may be introduced into mouse embryonic stem cells in addition to the human heavy and light chain polynucleotides. The mouse heavy and light chain immunoglobulin polynucleotides may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transpolynucleotides harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.), Genpharm (San Jose, Calif.), and Medarex, Inc. (Princeton, N.J.) can be engaged to provide human antibodies directed against a selected antigen using technology similar to that described above.

Similarly, human antibodies can be made by introducing human immunoglobulin loci into transgenic animals, e.g., mice in which the endogenous immunoglobulin polynucleotides have been partially or completely inactivated. Upon challenge, human antibody production is observed, which closely resembles that seen in humans in all respects, including polynucleotide rearrangement, assembly, and creation of an antibody repertoire. This approach is described, for example, in U.S. Pat. Nos. 5,545,807; 5,545,806; 5,569,825; 5,625,126; 5,633,425; 5,661,106, and in the following scientific publications: Marks et al., Biotechnol., 10:779-783 (1992); Lonberg et al., Nature 368:856-859 (1994); Fishwild et al., Nature Biotechnol., 14:845-51 (1996); Neuberger, Nature Biotechnol., 14:826 (1996); Lonberg and Huszer, Intern. Rev. Immunol., 13:65-93 (1995).

Completely human antibodies which recognize a selected epitope can be polynucleotiderated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, antibodies to the polypeptides of the invention can, in turn, be utilized to polynucleotiderate anti-idiotype antibodies that “mimic” polypeptides of the invention using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization and/or binding of a polypeptide of the invention to a ligand can be used to polynucleotiderate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

Such anti-idiotypic antibodies capable of binding to the HCLI polypeptide can be produced in a two-step procedure. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

The antibodies of the present invention may be bispecific antibodies. Bispecific antibodies are monoclonal, Preferably human or humanized, antibodies that have binding specificities for at least two different antigens. In the present invention, one of the binding specificities may be directed towards a polypeptide of the present invention, the other may be for any other antigen, and preferably for a cell-surface protein, receptor, receptor subunit, tissue-specific antigen, virally derived protein, virally encoded envelope protein, bacterially derived protein, or bacterial surface protein, etc.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transformed into a suitable host organism. For further details of polynucleotiderating bispecific antibodies see, for example Suresh et al., Meth. In Enzym., 121:210 (1986).

Heteroconjugate antibodies are also contemplated by the present invention. Heteroconjugate antibodies are composed of two covalently joined antibodies. Such antibodies have, for example, been proposed to target immune system cells to unwanted cells (U.S. Pat. No. 4, 676, 980), and for the treatment of HIV infection (WO 91/00360; WO 92/20373; and EP03089). It is contemplated that the antibodies may be prepared in vitro using known methods in synthetic protein chemistry, including those involving crosslinking agents. For example, immunotoxins may be constructed using a disulfide exchange reaction or by forming a thioester bond. Examples of suitable reagents for this purpose include iminothiolate and methyl-4-mercaptobutyrimidate and those disclosed, for example, in U.S. Pat. No. 4,676,980.

Polynucleotides Encoding Antibodies

The invention further provides polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody, preferably, that specifically binds to a polypeptide of the invention, preferably, an antibody that binds to a polypeptide having the amino acid sequence of SEQ ID NO: 2, 4, or 17.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be polynucleotiderated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library polynucleotiderated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular polynucleotide sequence to identify, e.g., a cDNA clone from a cDNA library that encodes the antibody. Amplified nucleic acids polynucleotiderated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutapolynucleotidesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties ), to polynucleotiderate antibodies having a different amino acid sequence, for example to create amino acid substitutions, deletions, and/or insertions.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and preferably human framework regions (see, e.g., Chothia et al., J. Mol. Biol. 278: 457-479 (1998) for a listing of human framework regions). Preferably, the polynucleotide polynucleotiderated by the combination of the framework regions and CDRs encodes an antibody that specifically binds a polypeptide of the invention. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to polynucleotiderate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing polynucleotides from a mouse antibody molecule of appropriate antigen specificity together with polynucleotides from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

More preferably, a clone encoding an antibody of the present invention may be obtained according to the method described in the Example section herein.

Methods of Producing Antibodies

The antibodies of the invention can be produced by any method known in the art for the synthesis of antibodies, in particular, by chemical synthesis or preferably, by recombinant expression techniques.

Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo polynucleotide recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early polynucleotide promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the polynucleotideration of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In polynucleotide, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target polynucleotide product can be released from the GST moiety.

In an insect system, Autographa californica nuclear polyhedrosis virus (AcNPV) is used as a vector to express foreign polynucleotides. The virus grows in Spodoptera frugiperda cells. The antibody coding sequence may be cloned individually into non-essential regions (for example the polyhedrin polynucleotide) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric polynucleotide may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the polynucleotide product in the specific fashion desired. Such modifications (e.g., glycosylation) and processing (e.g., cleavage) of protein products may be important for the function of the protein. Different host cells have characteristic and specific mechanisms for the post-translational processing and modification of proteins and polynucleotide products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the polynucleotide product may be used. Such mammalian host cells include but are not limited to CHO, VERY, BHK, Hela, COS, MDCK, 293, 3T3, W138, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) polynucleotides can be employed in tk-, hgprt- or aprt-cells, respectively. Also, antimetabolite resistance can be used as the basis of selection for the following polynucleotides: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, 1993, TIB TECH 11(5):155-215); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on polynucleotide amplification for the expression of cloned polynucleotides in mammalian cells in DNA cloning, Vol.3. (Academic Press, New York, 1987)). When a marker in the vector system expressing antibody is amplifiable, increase in the level of inhibitor present in culture of host cell will increase the number of copies of the marker polynucleotide. Since the amplified region is associated with the antibody polynucleotide, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalently and non-covalently conjugations) to a polypeptide (or portion thereof, preferably at least 10, 20, 30, 40, 50, 60, 70, 80, 90 or 100 amino acids of the polypeptide) of the present invention to polynucleotiderate fusion proteins. The fusion does not necessarily need to be direct, but may occur through linker sequences. The antibodies may be specific for antigens other than polypeptides (or portion thereof, preferably at least 10, 20, 30, 40, 50, 60, 70, 80, 90 or 100 amino acids of the polypeptide) of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al., supra, and PCT publication WO 93/21232; EP 439,095; Naramura et al., Immunol. Lett. 39:91-99 (1994); U.S. Pat. No.5,474,981; Gillies et al., PNAS 89:1428-1432 (1992); Fell et al., J. Immunol. 146:2446-2452(1991), which are incorporated by reference in their entireties.

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the constant region, hinge region, CH1 domain, CH2 domain, and CH3 domain or any combination of whole domains or portions thereof. The polypeptides may also be fused or conjugated to the above antibody portions to form multimers. For example, Fe portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88:10535-10539 (1991); Zheng et al., J. Immunol. 154:5590-5600 (1995); and Vil et al., Proc. Natl. Acad. Sci. USA 89:11337-11341(1992) (said references incorporated by reference in their entireties).

As discussed, supra, the polypeptides corresponding to a polypeptide, polypeptide fragment, or a variant of SEQ ID NO: 2, 4, or 17 may be fused or conjugated to the above antibody portions to increase the in vivo half life of the polypeptides or for use in immunoassays using methods known in the art. Further, the polypeptides corresponding to SEQ ID NO: 2, 4, or 17 may be fused or conjugated to the above antibody portions to facilitate purification. One reported example describes chimeric proteins consisting of the first two domains of the human CD4-polypeptide and various domains of the constant regions of the heavy or light chains of mammalian immunoglobulins. (EP 394,827; Traunecker et al., Nature 331:84-86 (1988). The polypeptides of the present invention fused or conjugated to an antibody having disulfide-linked dimeric structures (due to the IgG) may also be more efficient in binding and neutralizing other molecules, than the monomeric secreted protein or protein fragment alone. (Fountoulakis et al., J. Biochem. 270:3958-3964 (1995)). In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as h IL-5, have been fused with Fc portions for the purpose of high-throughput screening assays to identify antagonists of hIL-5. (See, Bennett et al., J. Molecular Recognition 8:52-58 (1995); Johanson et al., J. Biol. Chem. 270:9459-9471 (1995).

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

The present invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, 111In or 99Tc.

Further, an antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, β-interferon, nerve growth factor, platelet derived growth factor, tissue plasminogen activator, an apoptotic agent, e.g., TNF-alpha, TNF-beta, AIM I (See, International Publication No. WO 97/33899), AIM II (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-1”) interleukin-2 (“IL-2”), interleukin-6 (“IL-6”), granulocyte macrophage colony stimulating factor (“GM-CSF”), granulocyte colony stimulating factor (“G-CSF”), or other growth factors.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980, which is incorporated herein by reference in its entirety.

An antibody, with or without a therapeutic moiety conjugated to it, administered alone or in combination with cytotoxic factor(s) and/or cytokine(s) can be used as a therapeutic.

The present invention also encompasses the creation of synthetic antibodies directed against the polypeptides of the present invention. One example of synthetic antibodies is described in Radrizzani, M., et al., Medicina, (Aires), 59(6):753-8, (1999)). Recently, a new class of synthetic antibodies has been described and are referred to as molecularly imprinted polymers (MIPs) (Semorex, Inc.). Antibodies, peptides, and enzymes are often used as molecular recognition elements in chemical and biological sensors. However, their lack of stability and signal transduction mechanisms limits their use as sensing devices. Molecularly imprinted polymers (MIPs) are capable of mimicking the function of biological receptors but with less stability constraints. Such polymers provide high sensitivity and selectivity while maintaining excellent thermal and mechanical stability. MIPs have the ability to bind to small molecules and to target molecules such as organics and proteins' with equal or greater potency than that of natural antibodies. These “super” MIPs have higher affinities for their target and thus require lower concentrations for efficacious binding.

During synthesis, the MIPs are imprinted so as to have complementary size, shape, charge and functional groups of the selected target by using the target molecule itself (such as a polypeptide, antibody, etc.), or a substance having a very similar structure, as its “print” or “template.” MIPs can be derivatized with the same reagents afforded to antibodies. For example, fluorescent ‘super’ MIPs can be coated onto beads or wells for use in highly sensitive separations or assays, or for use in high throughput screening of proteins.

Moreover, MIPs based upon the structure of the polypeptide(s) of the present invention may be useful in screening for compounds that bind to the polypeptide(s) of the invention. Such a MIP would serve the role of a synthetic “receptor” by minimicking the native architecture of the polypeptide. In fact, the ability of a MIP to serve the role of a synthetic receptor has already been demonstrated for the estrogen receptor (Ye, L., Yu, Y., Mosbach, K, Analyst., 126(6):760-5, (2001); Dickert, F, L., Hayden, O., Halikias, K, P, Analyst., 126(6):766-71, (2001)). A synthetic receptor may either be mimicked in its entirety (e.g., as the entire protein), or mimicked as a series of short peptides corresponding to the protein (Rachkov, A., Minoura, N, Biochim, Biophys, Acta., 1544(1-2):255-66, (2001)). Such a synthetic receptor MIPs may be employed in any one or more of the screening methods described elsewhere herein.

MIPs have also been shown to be useful in “sensing” the presence of its mimicked molecule (Cheng, Z., Wang, E., Yang, X, Biosens, Bioelectron., 16(3):179-85, (2001); Jenkins, A, L., Yin, R., Jensen, J. L, Analyst., 126(6):798-802, (2001); Jenkins, A, L., Yin, R., Jensen, J. L, Analyst., 126(6):798-802, (2001)). For example, a MIP designed using a polypeptide of the present invention may be used in assays designed to identify, and potentially quantitate, the level of said polypeptide in a sample. Such a MIP may be used as a substitute for any component described in the assays, or kits, provided herein (e.g., ELISA, etc.).

A number of methods may be employed to create MIPs to a specific receptor, ligand, polypeptide, peptide, organic molecule. Several preferred methods are described by Esteban et al. in J. Anal, Chem., 370(7):795-802, (2001), which is hereby incorporated herein by reference in its entirety in addition to any references cited therein. Additional methods are known in the art and are encompassed by the present invention, such as for example, Hart, B, R., Shea, K, J. J. Am. Chem, Soc., 123(9):2072-3, (2001); and Quaglia, M., Chenon, K., Hall, A, J., De, Lorenzi, E., Sellergren, B, J. Am. Chem, Soc., 123(10):2146-54, (2001); which are hereby incorporated by reference in their entirety herein.

Uses for Antibodies directed against polypeptides of the invention

The antibodies of the present invention have various utilities. For example, such antibodies may be used in diagnostic assays to detect the presence or quantification of the polypeptides of the invention in a sample. Such a diagnostic assay may be comprised of at least two steps. The first, subjecting a sample with the antibody, wherein the sample is a tissue (e.g., human, animal, etc.), biological fluid (e.g., blood, urine, sputum, semen, amniotic-fluid, saliva, etc.), biological extract (e.g., tissue or cellular homogenate, etc.), a protein microchip (e.g., See Arenkov P, et al., Anal Biochem., 278(2):123-131 (2000)), or a chromatography column, etc. And a second step involving the quantification of antibody bound to the substrate. Alternatively, the method may additionally involve a first step of attaching the antibody, either covalently, electrostatically, or reversibly, to a solid support, and a second step of subjecting the bound antibody to the sample, as defined above and elsewhere herein.

Various diagnostic assay techniques are known in the art, such as competitive binding assays, direct or indirect sandwich assays and immunoprecipitation assays conducted in either heteropolynucleotideous or homogenous phases (Zola, Monoclonal Antibodies: A Manual of Techniques, CRC Press, Inc., (1987), pp147-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et a]., Nature, 144:945 (1962); Dafvid et al., Biochem., 13:1014 (1974); Pain et al., J. Immunol. Metho., 40:219(1981); and Nygren, J. Histochem. And Cytochem., 30:407 (1982).

Antibodies directed against the polypeptides of the present invention are useful for the affinity purification of such polypeptides from recombinant cell culture or natural sources. In this process, the antibodies against a particular polypeptide are immobilized on a suitable support, such as a Sephadex resin or filter paper, using methods well known in the art. The immobilized antibody then is contacted with a sample containing the polypeptides to be purified, and thereafter the support is washed with a suitable solvent that will remove substantially all the material in the sample except for the desired polypeptides, which are bound to the immobilized antibody. Finally, the support is washed with another suitable solvent that will release the desired polypeptide from the antibody.

Immunophenotyping

The antibodies of the invention may be utilized for immunophenotyping of cell lines and biological samples. The translation product of the polynucleotide of the present invention may be useful as a cell specific marker, or more specifically as a cellular marker that is differentially expressed at various stages of differentiation and/or maturation of particular cell types. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al., Cell, 96:737-49 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

Assays For Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols polynucleotidely comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, 1994, Current Protocols in Molecular Biology, Vol. 1, John Wiley & Sons, Inc., New York at 10.16.1.

Western blot analysis polynucleotidely comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 125I) in the presence of increasing amounts of an unlabeled second antibody.

Therapeutic Uses Of Antibodies

The present invention is further directed to antibody-based therapies which involve administering antibodies of the invention to an animal, preferably a mammal, and most preferably a human, patient for treating one or more of the disclosed diseases, disorders, or conditions. Therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant expression and/or activity of a polypeptide of the invention, including, but not limited to, any one or more of the diseases, disorders, or conditions described herein. The treatment and/or prevention of diseases, disorders, or conditions associated with aberrant expression and/or activity of a polypeptide of the invention includes, but is not limited to, alleviating symptoms associated with those diseases, disorders or conditions. Antibodies of the invention may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

The antibodies of the invention may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against polypeptides or polynucleotides of the present invention, fragments or regions thereof, for both immunoassays directed to and therapy of disorders related to polynucleotides or polypeptides, including fragments thereof, of the present invention. Such antibodies, fragments, or regions, will preferably have an affinity for polynucleotides or polypeptides of the invention, including fragments thereof. Preferred binding affinities include those with a dissociation constant or Kd less than 5×10-2 M, 10-2 M, 5×10-3 M, 10-3 M, 5×10-4 M, 10-4 M, 5×10-5 M, 10-5 M, 5×10-6 M, 10-6 M, 5×10-7 M, 10-7 M, 5×10-8 M, 10-8 M, 5×10-9 M, 10-9 M, 5×10-10 M, 10-10 M, 5×10-11 M, 10-11 M, 5×10-12 M, 10-12 M, 5×10-13 M, 10-13 M, 5×10-14 M, 10-14 M, 5×10-15 M, and 10-15 M.

Antibodies directed against polypeptides of the present invention are useful for inhibiting allergic reactions in animals. For example, by administering a therapeutically acceptable dose of an antibody, or antibodies, of the present invention, or a cocktail of the present antibodies, or in combination with other antibodies of varying sources, the animal may not elicit an allergic response to antigens.

Likewise, one could envision cloning the polynucleotide encoding an antibody directed against a polypeptide of the present invention, said polypeptide having the potential to elicit an allergic and/or immune response in an organism, and transforming the organism with said antibody polynucleotide such that it is expressed (e.g., constitutively, inducibly, etc.) in the organism. Thus, the organism would effectively become resistant to an allergic response resulting from the ingestion or presence of such an immune/allergic reactive polypeptide. Moreover, such a use of the antibodies of the present invention may have particular utility in preventing and/or ameliorating autoimmune diseases and/or disorders, as such conditions are typically a result of antibodies being directed against endogenous proteins. For example, in the instance where the polypeptide of the present invention is responsible for modulating the immune response to auto-antigens, transforming the organism and/or individual with a construct comprising any of the promoters disclosed herein or otherwise known in the art, in addition, to a polynucleotide encoding the antibody directed against the polypeptide of the present invention could effective inhibit the organisms immune system from eliciting an immune response to the auto-antigen(s). Detailed descriptions of therapeutic and/or polynucleotide therapy applications of the present invention are provided elsewhere herein.

Alternatively, antibodies of the present invention could be produced in a plant (e.g., cloning the polynucleotide of the antibody directed against a polypeptide of the present invention, and transforming a plant with a suitable vector comprising said polynucleotide for constitutive expression of the antibody within the plant), and the plant subsequently ingested by an animal, thereby conferring temporary immunity to the animal for the specific antigen the antibody is directed towards (See, for example, U.S. Pat. Nos. 5,914,123 and 6,034,298).

In another embodiment, antibodies of the present invention, preferably polyclonal antibodies, more preferably monoclonal antibodies, and most preferably single-chain antibodies, can be used as a means of inhibiting polynucleotide expression of a particular polynucleotide, or polynucleotides, in a human, mammal, and/or other organism. See, for example, International Publication Number WO 00/05391, published Feb. 3, 2000, to Dow Agrosciences LLC. The application of such methods for the antibodies of the present invention are known in the art, and are more particularly described elsewhere herein.

In yet another embodiment, antibodies of the present invention may be useful for multimerizing the polypeptides of the present invention. For example, certain proteins may confer enhanced biological activity when present in a multimeric state (i.e., such enhanced activity may be due to the increased effective concentration of such proteins whereby more protein is available in a localized location).

Antibody-Based Gene Therapy

In a specific embodiment, nucleic acids comprising sequences encoding antibodies or functional derivatives thereof, are administered to treat, inhibit or prevent a disease or disorder associated with aberrant expression and/or activity of a polypeptide of the invention, by way of polynucleotide therapy. Gene therapy refers to therapy performed by the administration to a subject of an expressed or expressible nucleic acid. In this embodiment of the invention, the nucleic acids produce their encoded protein that mediates a therapeutic effect.

Any of the methods for polynucleotide therapy available in the art can be used according to the present invention. Exemplary methods are described below.

For polynucleotide reviews of the methods of polynucleotide therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the compound comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo polynucleotide therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a polynucleotide gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contains nucleic acid sequences encoding an antibody of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the antibody to be used in polynucleotide therapy are cloned into one or more vectors, which facilitates delivery of the polynucleotide into a patient. More detail about retroviral vectors can be found in Boesen et al., Biotherapy 6:291-302 (1994), which describes the use of a retroviral vector to deliver the mdr1 polynucleotide to hematopoietic stem cells in order to make the stem cells more resistant to chemotherapy. Other references illustrating the use of retroviral vectors in polynucleotide therapy are: Clowes et al., J. Clin. Invest. 93:644-651 (1994); Kiem et al., Blood 83:1467-1473 (1994); Salmons and Gunzberg, Human Gene Therapy 4:129-141 (1993); and, Grossman and Wilson, Curr. Opin. in Genetics and Devel. 3:110-114 ( (1993).

Adenoviruses are other viral vectors that can be used in polynucleotide therapy. Adenoviruses are especially attractive vehicles for delivering polynucleotides to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based polynucleotide therapy. Bout et al., Human Gene Therapy 5:3-10 (1994) demonstrated the use of adenovirus vectors to transfer polynucleotides to the respiratory epithelia of rhesus monkeys. Other instances of the use of adenoviruses in polynucleotide therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in polynucleotide therapy (Walsh et al., Proc. Soc. Exp. Biol. Med. 204:289-300 (1993); U.S. Pat. No. 5,436,146).

Another approach to polynucleotide therapy involves transferring a polynucleotide to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred polynucleotide. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated polynucleotide transfer, microcell-mediated polynucleotide transfer, spheroplast fusion, etc. Numerous techniques are known in the art for the introduction of foreign polynucleotides into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoictic stem or progenitor cells) are preferably administered intravenously. The amount of cells envisioned for use depends on the desired effect, patient state, etc., and can be determined by one skilled in the art.

Cells into which a nucleic acid can be introduced for purposes of polynucleotide therapy encompass any desired, available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for polynucleotide therapy is autologous to the patient.

In an embodiment in which recombinant cells are used in polynucleotide therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

In a specific embodiment, the nucleic acid to be introduced for purposes of polynucleotide therapy comprises an inducible promoter operably linked to the coding region, such that expression of the nucleic acid is controllable by controlling the presence or absence of the appropriate inducer of transcription. Demonstration of Therapeutic or Prophylactic Activity.

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

Therapeutic/Prophylactic Administration and Compositions

The invention provides methods of treatment, inhibition and prophylaxis by administration to a subject of an effective amount of a compound or pharmaceutical composition of the invention, preferably an antibody of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see polynucleotidely ibid.)

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a polynucleotide gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Fcderal or a state government or listed in the U.S. Pharmacopeia or other polynucleotidely recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington'sPharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient'scircumstances. Effective doses may be extrapolated from dose-response curves derived from in vitro or animal model test systems.

For antibodies, the dosage administered to a patient is typically 0.1 mg/kg to 100 mg/kg of the patient'sbody weight. Preferably, the dosage administered to a patient is between 0.1 mg/kg and 20 mg/kg of the patient'sbody weight, more preferably 1 mg/kg to 10 mg/kg of the patient'sbody weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Diagnosis and Imaging with Antibodies

Labeled antibodies, and derivatives and analogs thereof, which specifically bind to a polypeptide of interest . can be used for diagnostic purposes to detect, diagnose, or monitor diseases, disorders, and/or conditions associated with the aberrant expression and/or activity of a polypeptide of the invention. The invention provides for the detection of aberrant expression of a polypeptide of interest, comprising (a) assaying the expression of the polypeptide of interest in cells or body fluid of an individual using one or more antibodies specific to the polypeptide interest and (b) comparing the level of polynucleotide expression with a standard polynucleotide expression level, whereby an increase or decrease in the assayed polypeptide polynucleotide expression level compared to the standard expression level is indicative of aberrant expression.

The invention provides a diagnostic assay for diagnosing a disorder, comprising (a) assaying the expression of the polypeptide of interest in cells or body fluid of an individual using one or more antibodies specific to the polypeptide interest and (b) comparing the level of polynucleotide expression with a standard polynucleotide expression level, whereby an increase or decrease in the assayed polypeptide polynucleotide expression level compared to the standard expression level is indicative of a particular disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell . Biol. 105:3087-3096 (1987)). Other antibody-based methods useful for detecting protein polynucleotide expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (112In), and technetium (99Tc); luminescent labels, such as lumino l; and fluorescent labels, such as fluorescein and rhodamine, and biotin.

One aspect of the invention is the detection and diagnosis of a disease or disorder associated with aberrant expression of a polypeptide of interest in an animal, preferably a mammal and most preferably a human. In one embodiment, diagnosis comprises: a) administering (for example, parenterally, subcutaneously, or intraperitoneally) to a subject an effective amount of a labeled molecule which specifically binds to the polypeptide of interest; b) waiting for a time interval following the administering for permitting the labeled molecule to preferentially concentrate at sites in the subject where the polypeptide is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of the polypeptide of interest. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99 mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

Kits

The present invention provides kits that can be used in the above methods. In one embodiment, a kit comprises an antibody of the invention, preferably a purified antibody, in one or more containers. In a specific embodiment, the kits of the present invention contain a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another specific embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In a more specific embodiment the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or calorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods polynucleotidely include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit polynucleotidely includes a support with surface-bound recombinant antigens, and a reporter-labeled anti-human antibody for detecting surface-bound anti-antigen antibody.

Microarrays and Screening Assays

In another embodiment of the present invention, oligonucleotides, or longer fragments derived from the HCLI polynucleotide sequence described herein can be used as targets in a microarray. The microarray can be used to monitor the expression levels of large numbers of polynucleotides simultaneously (to produce a transcript image), and to identify polynucleotide variants, mutations and polymorphisms. This information may be used to determine polynucleotide function, to understand the polynucleotide basis of a disease, to diagnose disease, and to develop and monitor the activities of therapeutic agents. In a particular aspect, the microarray is prepared and used according to the methods described in WO 95/11995 (Chee et al.); D. J. Lockhart et al., 1996, Nature Biotechnology, 14:1675-1680; and M. Schena et al., 1996, Proc. Natl. Acad. Sci. USA, 93:10614-10619). Microarrays are further described in U.S. Pat. No. 6,015,702 to P. Lal et al.

In another embodiment of this invention, a nucleic acid sequence which encodes a novel HCLI polypeptide, may also be used to polynucleotiderate hybridization probes, which are useful for mapping the naturally occurring genomic sequence. The sequences may be mapped to a particular chromosome, to a specific region of a chromosome, or to artificial chromosome constructions (HACs), yeast artificial chromosomes (YACs), bacterial artificial chromosomes (BACs), bacterial PI constructions, or single chromosome cDNA libraries, as reviewed by C. M. Price, 1993, Blood Rev., 7:127-134 and by B. J. Trask, 1991, Trends Genet., 7:149-154.

In another embodiment of the present invention, an HCLI polypeptide of this invention, its catalytic or immunogenic fragments, or oligopeptides thereof, can be used for screening libraries of compounds in any of a variety of drug screening techniques. The fragment employed in such screening may be free in solution, affixed to a solid support, borne on a cell surface, or located intracellularly. The formation of binding complexes, between the HCLI polypeptide, or a portion thereof, and the agent being tested, may be measured utilizing techniques commonly practiced in the art.

Another technique for drug screening, which may be employed, provides for high throughput screening of compounds having suitable binding affinity to the protein of interest as described in WO 84/03564 (Venton, et al.). In this method, as applied to the HCLI protein, large numbers of different small test compounds are synthesized on a solid substrate, such as plastic pins or some other surface. The test compounds are reacted with the HCLI polypeptide, or fragments thereof, and washed. Bound HCLI polypeptide is then detected by methods well known in the art. Purified HCLI polypeptide can also be coated directly onto plates for use in the aforementioned drug screening techniques. Alternatively, non-neutralizing antibodies can be used to capture the peptide and immobilize it on a solid support.

In a further embodiment, competitive drug screening assays can be used in which neutralizing antibodies, capable of binding an HCLI polypeptide according to this invention, specifically compete with a test compound for binding to the HCLI polypeptide. In this manner, the antibodies can be used to detect the presence of any peptide that shares one or more antigenic determinants with the HCLI polypeptide.

The human HCLI polypeptides and/or peptides of the present invention, or immunogenic fragments or oligopeptides thereof, can be used for screening therapeutic drugs or compounds in a variety of drug screening techniques. The fragment employed in such a screening assay may be free in solution, affixed to a solid support, borne on a cell surface, or located intracellularly. The reduction or abolition of activity of the formation of binding complexes between the ion channel protein and the agent being tested can be measured. Thus, the present invention provides a method for screening or assessing a plurality of compounds for their specific binding affinity with a HCLI polypeptide, or a bindable peptide fragment, of this invention, comprising providing a plurality of compounds, combining the HCLI polypeptide, or a bindable peptide fragment, with each of a plurality of compounds for a time sufficient to allow binding under suitable conditions and detecting binding of the HCLI polypeptide or peptide to each of the plurality of test compounds, thereby identifying the compounds that specifically bind to the HCLI polypeptide or peptide.

Methods of identifying compounds that modulate the activity of the novel human HCLI polypeptides and/or peptides are provided by the present invention and comprise combining a potential or candidate compound or drug modulator of intracellular chloride ion channel biological activity with an HCLI polypeptide or peptide, for example, the HCLI amino acid sequence as set forth in SEQ ID NO: 2, and measuring an effect of the candidate compound or drug modulator on the biological activity of the HCLI polypeptide or peptide. Such measurable effects include, for example, physical binding interaction; the ability to cleave a suitable intracellular chloride ion channel substrate; effects on native and cloned HCLI-expressing cell line; and effects of modulators or other intracellular chloride ion channel-mediated physiological measures.

Another method of identifying compounds that modulate the biological activity of the novel HCLI polypeptides of the present invention comprises combining a potential or candidate compound or drug modulator of a intracellular chloride ion channel biological activity with a host cell that expresses the HCLI polypeptide and measuring an effect of the candidate compound or drug modulator on the biological activity of the HCLI polypeptide. The host cell can also be capable of being induced to express the HCLI polypeptide, e.g., via inducible expression. Physiological effects of a given modulator candidate on the HCLI polypeptide can also be measured. Thus, cellular assays for particular intracellular chloride ion channel modulators may be either direct measurement or quantification of the physical biological activity of the HCLI polypeptide, or they may be measurement or quantification of a physiological effect. Such methods preferably employ a HCLI polypeptide as described herein, or an overexpressed recombinant HCLI polypeptide in suitable host cells containing an expression vector as described herein, wherein the HCLI polypeptide is expressed, overexpressed, or undergoes upregulated expression.

Another aspect of the present invention embraces a method of screening for a compound that is capable of modulating the biological activity of a HCLI polypeptide, comprising providing a host cell containing an expression vector harboring a nucleic acid sequence encoding a HCLI polypeptide, or a functional peptide or portion thereof (e.g., SEQ ID NO: 2); determining the biological activity of the expressed HCLI polypeptide in the absence of a modulator compound; contacting the cell with the modulator compound and determining the biological activity of the expressed HCLI polypeptide in the presence of the modulator compound. In such a method, a difference between the activity of the HCLI polypeptide in the presence of the modulator compound and in the absence of the modulator compound indicates a modulating effect of the compound.

Essentially any chemical compound can be employed as a potential modulator or ligand in the assays according to the present invention. Compounds tested as intracellular chloride ion channel modulators can be any small chemical compound, or biological entity (e.g., protein, sugar, nucleic acid, lipid). Test compounds will typically be small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are typically run in parallel, for example, in microtiter formats on microtiter plates in robotic assays. There are many suppliers of chemical compounds, including Sigma (St. Louis, Mo.), Aldrich (St. Louis, Mo.), Sigma-Aldrich (St. Louis, Mo.), Fluka Chemika-Biochemica Analytika. (Buchs, Switzerland), for example. Also, compounds may be synthesized by methods known in the art.

High throughput screening methodologies are particularly envisioned for the detection of modulators of the novel HCLI polynucleotides and polypeptides described herein. Such high throughput screening methods typically involve providing a combinatorial chemical or peptide library containing a large number of potential therapeutic compounds (e.g., ligand or modulator compounds). Such combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

A combinatorial chemical library is a collection of diverse chemical compounds polynucleotiderated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without limitation, peptide libraries (e.g. U.S. Pat. No. 5,010,175; Furka, 1991, Int. J. Pept. Prot. Res., 37:487-493; and Houghton et al., 1991, Nature, 354:84-88). Other chemistries for polynucleotiderating chemical diversity libraries can also be used. Nonlimiting examples of chemical diversity library chemistries include, peptoids (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCT [US96/10287), carbohydrate libraries (e.g., Liang et al., 1996, Science, 274-1520-1522) and U.S. Pat. No. 5,593,853), small organic molecule libraries (e.g., benzodiazepines, Baum C&EN, Jan. 18, 1993, page 33; and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

Devices for the preparation of combinatorial libraries are commercially available (e.g., 357 MPS, 390 MPS, Advanced Chem Tech, Louisville Ky.; Symphony, Rainin, Woburn, MA; 433A Applied Biosystems, Foster City, Calif.; 9050 Plus, Millipore, Bedford, Mass.). In addition, a large number of combinatorial libraries are commercially available (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

In one embodiment, the invention provides solid phase based in vitro assays in a high throughput format, where the cell or tissue expressing an ion channel is attached to a solid phase substrate. In such high throughput assays, it is possible to screen up to several thousand different modulators or ligands in a single day. In particular, each well of a microtiter plate can be used to perform a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds. It is possible to assay several different plates per day; thus, for example, assay screens for up to about 6,000-20,000 different compounds are possible using the described integrated systems.

In another of its aspects, the present invention encompasses screening and small molecule (e.g., drug) detection assays which involve the detection or identification of small molecules that can bind to a given protein, i.e., a HCLI polypeptide or peptide. Particularly preferred are assays suitable for high throughput screening methodologies.

In such binding-based detection, identification, or screening assays, a functional assay is not typically required. All that is needed is a target protein, preferably substantially purified, and a library or panel of compounds (e.g., ligands, drugs, small molecules) or biological entities to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al.; see also, J. Zimmerman, 2000, Gen. Eng. News, 20(8)). The assay allows the detection of small molecules (e.g., drugs, ligands) that bind to expressed, and preferably purified, ion channel polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

To purify a HCLI polypeptide or peptide to measure a biological binding or ligand binding activity, the source may be a whole cell lysate that can be prepared by successive freeze-thaw cycles (e.g., one to three) in the presence of standard protease inhibitors. The HCLI polypeptide may be partially or completely purified by standard protein purification methods, e.g., affinity chromatography using specific antibody described infra, or by ligands specific for an epitope tag engineered into the recombinant HCLI polypeptide molecule, also as described herein. Binding activity can then be measured as described.

Compounds which are identified according to the methods provided herein, and which modulate or regulate the biological activity or physiology of the HCLI polypeptides according to the present invention are a preferred embodiment of this invention. It is contemplated that such modulatory compounds may be employed in treatment and therapeutic methods for treating a condition that is mediated by the novel HCLI polypeptides by administering to an individual in need of such treatment a therapeutically effective amount of the compound identified by the methods described herein.

In addition, the present invention provides methods for treating an individual in need of such treatment for a disease, disorder, or condition that is mediated by the HCLI polypeptides of the invention, comprising administering to the individual a therapeutically effective amount of the HCLI-modulating compound identified by a method provided herein.

### EXAMPLES

The Examples herein are meant to exemplify the various aspects of carrying out the invention and are not intended to limit the scope of the invention in any way. The Examples do not include detailed descriptions for conventional methods employed, such as in the construction of vectors, the insertion of cDNA into such vectors, or the introduction of the resulting vectors into the appropriate host. Such methods are well known to those skilled in the art and are described in numerous publications, for example, Sambrook, Fritsch, and Maniatis, Molecular Cloning: A Laboratory Manual, 2^{nd} Edition, Cold Spring Harbor Laboratory Press, USA, (1989).

### Example 1

Bioinformations Ananlysis

Currently, one approach used for identifying and characterizing the polynucleotides distributed along the human genome includes utilizing large fragments of genomic DNA which are isolated, cloned, and sequenced. Potential open reading frames in these genomic sequences were identified using bioinformatics software.

Ion channel sequences were used as probes to search the human genomic sequence database. The search program used was BLAST2.0 (S. F. Altschul et al., 1997, Nucl. Acid. Res., 25:3389-3402). Ion channel specific Hidden Markov Models (HMMs) built in-house or obtained from the public PFAM databases were also used as probes (Bateman, A. et al., 2000, Nucl. Acid. Res., 28:263-266). The search program used for HMMs was the Genewise/Wise 2 package (http://www.sanger.ac.uk/Software/Wise2/idenx.shtmi). The top genomic exon hits from the results were searched back against the non-redundant protein and patent sequence databases. From this analysis, exons encoding novel potential ion channels were identified based on sequence homology. Also, the genomic region surrounding the matching exons was analyzed. Based on this analysis, the full length nucleotide sequence (SEQ ID NO: 1, FIGS. 1A-D) of the novel human ion channel related polynucleotide, HCLI, was experimentally obtained.

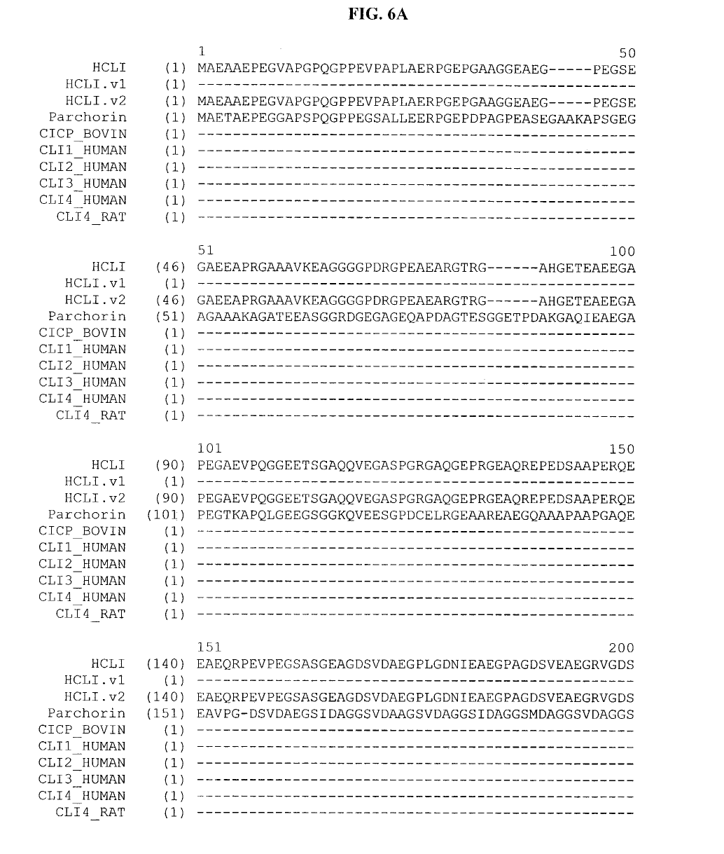

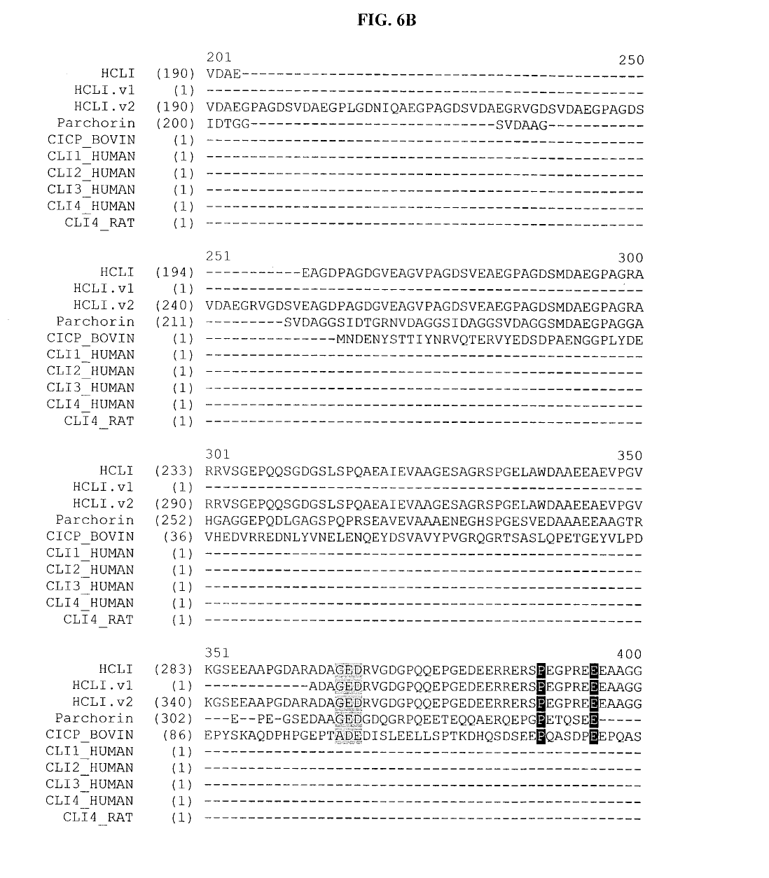

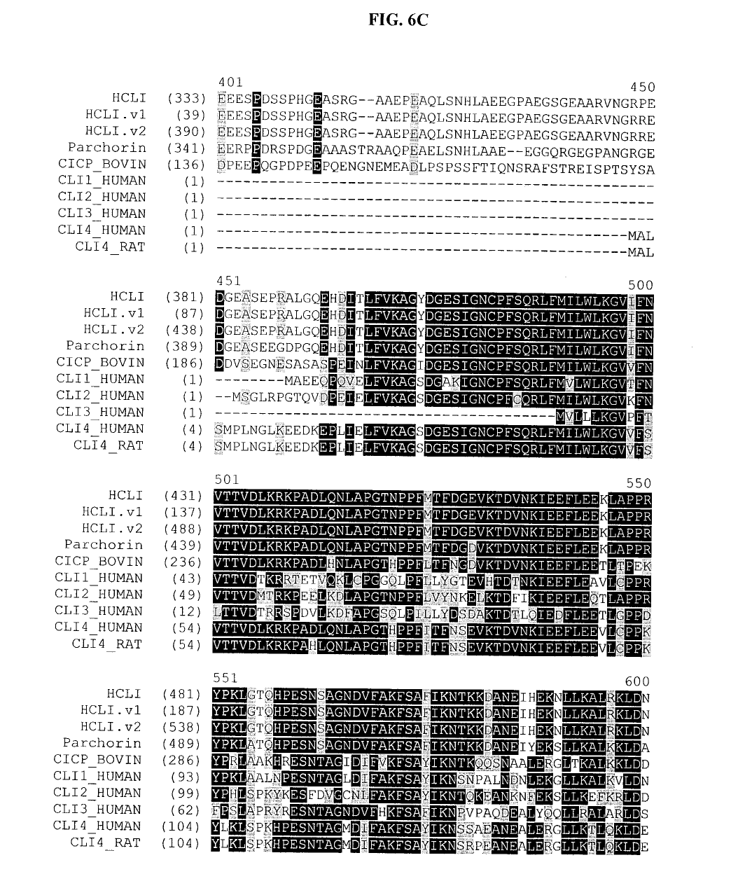

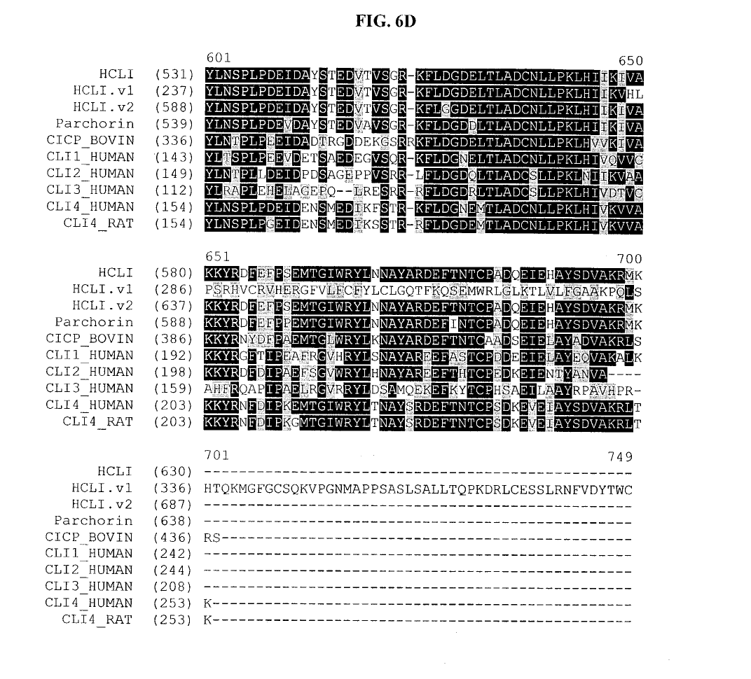

The amino acid sequence of the HCLI polypeptide (SEQ ID NO: 2) encoded by the HCLI polynucleotide sequence (SEQ ID NO: 1) was searched using the BLAST2.0 program against the non-redundant protein and patent sequence databases. The alignments of the HCLI (SEQ ID NO: 2) polypeptide sequence with the top matching hit and with the HCLI variants was performed using the GAP and GCG pileup programs. The amino acid sequences on the top lines represent the novel HCLI amino acid sequence of the invention. The amino acid sequences on the bottom lines represent the local matching sequence of either the HCLI variant (SEQ ID NO: 4) in FIGS. 2A-B, or the top-matching protein (Parchorin, SEQ ID NO: 5) in FIGS. 5A-5B. Vertical dashes between the top and bottom sequence lines represent identical amino acids between the two sequences. Two vertical dots between the top and bottom sequence lines represent similar amino acids between the two sequences. The GAP program polynucleotiderates percent identity/similarity using an alogrithm based on the following paper: Needleman, S. B., Wunsch, C. D. (1970) J. of Mol. Biol., 48(3):443-53. The GAP program polynucleotiderated 71 % identity and 75% similarity values between HCLI and Parchorin. These results indicate that the HCLI polypeptide of this invention represents a novel member of the chloride channel protein family (see also multiple sequence alignment, FIGS. 6A-6D), and in particular, is most similar to a intracellular chloride ion channel-related protein. Furthermore, the alignment between HCLI and the HCLI variants indicates that the two proteins share a high degree of local identity (80% and 91%, for HCLI.v1 and HCLI.v2, respectively), and that the HCLI.v1 variant contains an alternate C-terminus. It is thus expected that the HCLI, HCLI.v1, and HCLI.v2 variant polypeptides share biological activity with members of the chloride ion channel family, in addition to specific members known in the art, or as otherwise described herein.

The multiple sequence alignment (FIGS. 6A-6D) of HCLI polypeptide sequence (SEQ ID NO: 2), and its variants, HCLI.v1 (SEQ ID NO: 4), and HCLI.v2 (SEQ ID NO: 17), with the top matching hits was also performed using the CLUSTALW algorithm described elsewhere herein, as available within the Vector NTI AlignX program (CLUSTALW parameters: gap opening penalty: 10; gap extension penalty: 0.5; gap separation penalty range: 8; percent identity for alignment delay: 40%; and transition weighting: 0). The darkly shaded amino acids represent regions of matching identity. The lightly shaded amino acids represent regions of matching similarity. Lines between residues indicate gapped regions for the aligned polypeptides. A dendrogram summarizing the similarity relationships between these sequences can be seen in FIG. 7. Within the dendrogram, a closer vertical distance between proteins indicates higher amino acid sequence similarity, and a greater vertical distance between proteins indicates less similarity.

The sequence information from the novel polynucleotide candidates is used for full-length cloning and expression profiling. Primer sequences are obtained using the primer3 program (Steve Rozen, Helen J. Skaletsky (1996,1997) Primer3. Code available at http://www-genome.wi.mit.edu/genome₁₃ software/other/primer3.html). The HCLI polynucleotide specific primers (SEQ ID NOS: 13-14) were used in the cloning process and the “internal oligo” (SEQ ID NO: 12) was used as a hybridization probe to detect the PCR product after amplification.

### Example 2

Multiplex Cloning of HCLI cDNA

General Strategy

Using bioinformatic predicted polynucleotide sequence, the following types of polynucleotide-specific PCR primers and cloning oligos are designed: ‘A’ type PCR primer pairs that reside within a single predicted exon, ‘B’ type PCR primer pairs that cross putative exon/intron boundaries, and ‘C’ type, 80 mer antisense and sense oligos containing a biotin moiety on its 5′ end. The primer pairs from the A type are optimized on human genomic DNA, and the B type on a mixture of first strand cDNAs made with and without reverse transcriptase, from brain and testis poly A+ RNA. The information obtained with the B type primers is used to assess which putative expressed sequences can be experimentally observed to have reverse transcriptase dependent expression. The primer pairs from the A type are less stringent in terms of identifying expressed sequences, but because they amplify genomic DNA as well as cDNA, the ability to amplify genomic DNA provides for the necessary positive control for the primer pair. Negative results with the B type are subjected to the caveat that the first strand sequence may not be expressed in the tissue that is under examination, and without a positive control, a negative result is meaningless.

The biotinylated 80 mer oligos are added en mass to pools of single strand cDNA libraries. Up to 50 probes have been successfully used on pools for 15 different libraries. The orientation of the oligo depends on the orientation of the cDNA in its vector. Antisense 80 mer oligos are used for those libraries and cloned into pCMVSPORT and pSPORT whereas sense 80 mer oligos are used for cDNA libraries cloned into pSPORT2. After the primary selection is carried out, all of the captured DNA is repaired to double strand form using the T7 primer for the commercial libraries in pCMVSPORT, and the Sp6 primer for in-house constructed libraries in pSPORT. The resulting DNA is electroporated into E. coli DH12S and plated onto 150 mm plates with nylon filters. The cells are scraped and a frozen stock is made. This is the primary selected library. One-fifth of the library is polynucleotidely converted into single strand form and the DNA assayed with the polynucleotide specific primers pairs (GSPs). The next round of solution hybridization capture is carried out with 80 mer oligos for only those sequences that were positive with the polynucleotides-specific-primers. After the second round, the captured single strand DNAs are repaired with a pool of GSPs, where only the primer complementary to polarity of the single-stranded circular DNA is used (the antisense primer for pCMVSPORT and pSPORT1 and the sense primer for pSPORT2). The resulting colonies are screened by PCR using the GSPs. Typically, greater than 80% of the clones are positive for any given GSP. The entire 96 well block of clones are min-prep and each of clones sized by either PCR or restriction enzyme digestion. A selection of different size clones for each targeted sequence are chosen for transposon-hopping and DNA sequencing.

Success of the method, like any cDNA cloning method, depends on the quality of the libraries employed. High complexity and large average insert size are required. We have employed HPLC as a means of fractionating cDNA for the purpose of constructing libraries.

A. Construction of size Fractionated Brain and Testis cDNA Libraries

Brain and testis polyA+ RNA were purchased from Clontech, treated with DNase I to remove traces of genomic DNA contamination and converted into double stranded cDNA using the SuperScript™ Plasmid System for cDNA Synthesis and Plasmid Cloning (Life Technologies). No radioisotope was incorporated in either of the cDNA synthesis steps. The cDNA was then size fractionated on a TransGenomics HPLC system equipped with a size exclusion column (TosoHass) with dimensions of 7.8 mm×30 cm and a particle size of 10 μm. Tris buffered saline (TBS) was used as the mobile phase, and the column was run at a flow rate of 0.5 mL/min. The resulting chromatograms were analyzed to determine which fractions should be pooled to obtain the largest cDNA's; polynucleotidely fractions that eluted in the range of 12 to 15 minutes were pooled.

The cDNA was precipitated, concentrated and then ligated into the SalI/NotI sites in pSPORT1 vector. After electroporation into E. coli strain DH12 S, using a combination of PCR primers to the ends of the vector and Sal I/Not I restriction enzyme digestion of mini-prep DNA, it was determined that the average insert size of libraries made in this fashion was greater the 3.5 Kb; the overall complexity of the library was greater than 10⁷ independent clones. The library was amplified in semi-solid agar for 2 days at 30° C. An aliquot (200 microliters) of the amplified library was inoculated into a 200 mL culture for single-stranded DNA isolation by super-infection with an f1 helper phage. After an overnight growth, the released phage particles were precipitated with PEG and the single stranded circular DNA was concentrated by ethanol precipitation, resuspended at a concentration of one microgram per microliter and used for the cDNA capture experiments.

B. Conversion of Double-Stranded cDNA Libraries into Single-Stranded Circular form

To prepare cultures, 200 mL LB with 400 μL carbenicillin (100 mg/mL stock solution) were inoculated with from 200 μL to 1 mL of thawed cDNA library and incubated at 37° C. while shaking at 250 rpm for approximately 45 minutes, or until an OD600 of 0.025-0.040 was attained. M13K07 helper phage (1 mL) was added to the culture and grown for 2 hours, after which Kanamycin (500 μl; 30 mg/mL) was added and the culture was grown for an additional 15-18 hours.

The culture was then poured into 6 screw-cap tubes (50 mL autoclaved tubes) and cells subjected to centrifugation at 10K in an HB-6 rotor for 15 minutes at 4° C. to pellet the cells. The supernatant was filtered through a 0.2 μm filter and 12,000 units of Gibco DNase I was added. The mixture was incubated for 90 minutes at room temperature.

For PEG precipitation, 50 mL of ice-cold 40% PEG 8000, 2.5 M NaCl, and 10 mM MgSO₄ was added to the supernatant, mixed, and aliquotted into 6 centrifuge tubes (covered with parafilm). The tubes and contents were incubated for 1 hour on wet ice or at 4° C. overnight. The tubes were then centrifuged at 10K in a HB-6 rotor for 20 minutes at 4° C. to pellet the helper phage.

Following centrifugation, the supernatant was discarded and the sides of the tubes were dried. Each pellet was resuspended in 1 mL TE, pH 8. The resuspended pellets were pooled into a 14 mL tube (Sarstadt ), (6 mL total). SDS was added to 0.1% (60 μl of stock 10% SDS). Freshly made proteinase K (20 mg/mL) was added (60 μl ) and the suspension was incubated for 1 hour at 42° C.

For phenol/chloroform extractions, 1 mL of NaCl (5M) was added to the suspension in the tube. An equal volume of phenol/chloroform (6 mL) was added and the contents were vortexed or shaken. The suspension was then centrifuged at 5K in an HB-6 rotor for 5 minutes at 4° C. The aqueous (top) phase was transferred to a new tube (Sarstadt) and extractions were repeated until no interface is visible.

Ethanol precipitation was then performed on the aqueous phase whose volume is divided into 2 tubes (3 mL each). To each tube, 2 volumes of 100% ethanol were added and precipitation was carried out overnight at −20° C. The precipitated DNA was pelleted at 10K in an HB-6 rotor for 20 minutes at 4° C. The ethanol was discarded. Each pellet was resuspended in 700 μl of 70% ethanol. The contents of each tube were combined into one micro centrifuge tube and centrifuged in a micro centrifuge (Eppendorf) at 14K for 10 minutes at 4° C. After discarding the ethanol, the DNA pellet was dried in a speed vacuum. In order to remove oligosaccharides, the pellet was resuspended in 50 μl TE buffer, pH8. The resuspension was incubated on dry ice for 10 minutes and centrifuged at 14K in an Eppendorf microfuge for 15 minutes at 4° C. The supernatant was then transferred to a new tube and the final volume was recorded.

To check purity, DNA was diluted 1:100 and added to a micro quartz cuvette, where DNA was analyzed by spectrometry at an OD260/OD280. The preferred purity ratio is between 1.7 and 2.0. The DNA was diluted to 1 μg/μL in TE, pH8 and stored at 4° C. The concentration of DNA was calculated using the formula: (32 μg/mL*OD)(mL/1000 μL)(100)(OD260). The quality of single-stranded DNA was determined by first mixing 1 μL of 5 μg/μl ssDNA; 11 μL deionized water; 1.5 μL 10 μM T7 sport primer (fresh dilution of stock); 1.5 μl 10×Precision-Taq buffer per reaction. In the repair mix, a cocktail of 4 μl of 5 mM dNTPs (1.25 mM each); 1.5 μL 10× Precision-Taq buffer; 9.25 μL deionized water; and 0.25 μL Precision-Taq polymerase was mixed per reaction and preheated at 70° C. until the middle of the thermal cycle.

The DNA mixes were aliquotted into PCR tubes and the thermal cycle was started. The PCR thermal cycle consists of 1 cycle at 95° C. for 20 sec.; 59° C. for 1 min. (15 μL repair mix added); and 73° C. for 23 minutes. For ethanol precipitation, 15 μg glycogen, 16 μl ammonium acetate (7.5M), and 125 μL 100% ethanol were added and the contents were centrifuged at 14K in an Eppendorf microfuge for 30 minutes at 4° C. The resulting pellet was washed 1 time with 125 μL 70% ethanol and then the ethanol was discarded. The pellet was dried in a speed vacuum and resuspended in 10 μL TE buffer, pH 8.

Single-stranded DNA was electroporated into E. coli DH10B or DH12 S cells by pre-chilling the cuvettes and sliding holder, and thawing the cells on ice-water. DNA was aliquotted into micro centrifuge tubes (Eppendorf) as follows: 2 μL repaired library, (=1×10⁻³ μg); 1 μL unrepaired library (1 ng/μL), (=1×10⁻³ μg); and 1 μL pUC19 positive control DNA (0.01 μg/μL), (=1×10⁻⁵ μg). The mixtures were stored on ice until use.

One at a time, 40 μL of cells were added to a DNA aliquot. The cell/DNA mixture was not pipetted up and down more than one time. The mixture was then transferred via pipette into a cuvette between the metal plates, and electroporation was performed at 1.8 kV. Immediately afterward, 1 mL SOC medium (i.e., SOB (bacto-tryptone; bacto-yeast extract; NaCl)+glucose (20 mM)+Mg²⁺) (See, J. Sambrook et al., Molecular Cloning: A Laboratory Manual, 2nd Ed., A.2, 1989) was added to the cuvette and the contents were transferred into 15 mL of media, as commonly known in the art. The cells were allowed to recover for 1 hour at 37° C. with shaking (225 rpm).

Serial dilutions of the culture were made in 1:10 increments (20 μL into 180 μL LB) for plating the electroporated cells. For the repaired library, dilutions of 1:100, 1:1000, 1:10,000 were made. For the unrepaired library, dilutions of 1:10 and 1:100 were made. Positive control dilutions of 1:10 and 1:100 were made. Each dilution (100 μL) was plated onto small plates containing LB+carbenicillin and incubated at 37° C. overnight. The titer and background were calculated by methods well known in the art. Specifically, the colonies on each plate were counted using the lowest dilution countable. The titer was calculated using the formula: (# of colonies)(dilution factor)(200 μL/100 μL)(1000 μL/20 μL)=CFUs, where CFUs/μg DNA used=CFU/μg. The % background=((unrepaired CFU/μg)/(repaired CFU/μg))×100%.

C. Solution Hybridization and DNA Capture

One microliter of anti-sense biotinylated oligonucleotides (or sense oligonucleotides when annealing to single-stranded DNA from pSPORT2 vector) containing 150 ng of up to 50 different 80-mer oligonucleotide probes was added to 6 μL (6 μg) of a mixture of up to 15 single-stranded, covalently-closed, circular cDNA libraries and 7 μL of 100% formamide in a 0.5 mL PCR tube. The sequence of the 80 mer antisense oligo that was used in the present invention to clone HCLI is: 5′-biotin-aagaactcctcgatcttattcacatccgtcttgacttcaccatcaaaagtcatgaaaggagggtttgttccgggagc ag-3′ (SEQ ID NO: 12).

Thus, one microliter (150 ng) of SEQ ID NO: 12 was added to 6 μL (6 μg) of a single-stranded covalently closed circular testis cDNA library and 7 μL of 100% fornamide in a 0.5 ml PCR tube. The mixture was heated in a thermal cycler to 95° C. for 2 minutes. Fourteen microliters of 2× hybridization buffer (50% formamide, 1.5 M NaCl, 0.04 M NaPO₄, pH 7.2, 5 mM EDTA, 0.2% SDS) was added to the heated probe/cDNA library mixture and incubated at 42° C. for 26 hours. Hybrids between the biotinylated oligo and the circular cDNA was isolated by diluting the hybridization mixture to 220 microliters in a solution containing 1 M NaCl, 10 mM Tris-HCl pH 7.5, 1 mM EDTA, pH 8.0 and adding 125 microliters of streptavidin magnetic beads. This solution was incubated at 42° C. for 60 minutes, and mixed every 5 minutes to resuspend the beads. The beads were separated from the solution with a magnet and washed three times in 200 microliters of 0.1×SSPE, 0.1% SDS at 45° C.

The single stranded cDNAs were released from the biotinylated oligo/streptavidin magnetic bead complex by adding 50 microliters of 0.1 N NaOH and incubating at room temperature for 10 minutes. Six microliters of 3 M sodium acetate were added along with 15 μg of glycogen and the solution was ethanol precipitated with 120 microliters of 100% ethanol. The precipitated DNA was re-suspended in 12 μL of TE (10 mM Tris-HCl, pH 8.0, 1 mM EDTA, pH 8.0). The single stranded cDNA was converted into double strands in a thermal cycler by mixing 5 μL of the captured DNA with 1.5 μL of 10 μM of standard SP6 Sport primer: 5′-atttaggtgacactatag-3′ (SEQ ID NO: 15) (homologous to a sequence on the cDNA cloning vector), and 1.5 μL of 10×PCR buffer. The mixture was heated to 95° C. for 20 seconds, and then ramped down to 59° C. At this time 15 μL of a repair mix, preheated to 70° C., was added to the DNA (Repair mix contains 4 μL of 5 mM dNTPs (1.25 mM each), 1.5 μL of 10×PCR buffer, 9.25 μL of water, and 0.25 μL of Taq polymerase). The solution was ramped back to 73° C. and incubated for 23 minutes.

The repaired DNA was ethanol precipitated and resuspended in 10 μL of TE. Two μL were electroporated per tube containing 40 μL of E. coli DH12S cells. Three hundred and thirty three μL (333 μL) were plated onto one 150 mm plate of LB agar plus 100 μg/mL of ampicillin. After overnight incubation at 37° C., the colonies from all plates were harvested by scraping into 10 mL of LB+50 μg/mL of ampicillin and 2 mL of sterile glycerol.

The second round of selection was initiated by making single-strand circular DNA from the primary selected library using the above-described method. The purified single-stranded circular DNA was then assayed with HCLI polynucleotide-specific primers(GSPs), 5′-gagaaattagctcccccgag-3′ (SEQ ID NO: 13) and 5′-gcttgggtaagaggttgcag-3′ (SEQ ID NO: 14), using standard PCR conditions. The hybridization was performed including only those 80 mer biotinylated probes (for example, SEQ ID NO: 12) whose targeted sequences had a positive result with the GSPs. The resulting single-stranded circular DNA was converted into double strands using the antisense oligo for each target sequence as the repair primer (sense primers are used for material captured from pSPORT2 libraries). The resulting double stranded DNA was electroporated into DH10B cells and the resulting colonies were inoculated into 96 deep well blocks. After overnight growth, DNA was prepared and sequentially screened for each of the targeted sequences using the GSPs. The DNA was also digested with SalI and NotI restriction enzymes and the inserts were sized by agarose gel electrophoresis.

### Example 4

RNA Ligase Protocol for Generating the 5′ or 3′ end Sequences to Obtain the Full-Length HCLI Gene

Once an HCLI polynucleotide/polynucleotide sequence of interest is identified, several methods are available for the identification of 5′ or 3′ portions of the polynucleotide which may not be present in the original cDNA plasmid. These methods include, but are not limited to, filter probing, clone enrichment using specific probes and protocols similar and identical to 5′ and 3′RACE. While the full-length polynucleotide may be present in the library and can be identified by probing, a useful method for polynucleotiderating the 5′ or 3′ end is to use the existing sequence information from the original cDNA to polynucleotiderate the missing info r mation. A method similar to 5′RACE is available for polynucleotiderating the missing 5′ end of a desired full-length polynucleotide. (This method was published by Fromont-Racine et al., Nucleic Acids Res., 21(7): 1683-1684 (1993)).

Briefly, a specific RNA oligonucleotide is ligated to the 5′ ends of a population of RNA, preferably 30, containing full-length polynucleotide RNA transcripts and a primer set containing a primer specific to the ligated RNA oligonucleotide and a primer specific to a known sequence of the polynucleotide of interest, and is used to PCR amplify the 5′ portion of the desired full length polynucleotide which may then be sequenced and used to polynucleotiderate the full-length polynucleotide. This method starts with total RNA isolated from the desired source. PolyA RNA may be used, but is not a prerequisite for this procedure. The RNA preparation is then be treated with phosphatase if necessary to eliminate 5′ phosphate groups on degraded or damaged RNA which may interfere with the later RNA ligase step. The phosphatase, if used, is then inactivated and the RNA is treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase. This modified RNA preparation can then be used as a template for first strand cDNA synthesis using a polynucleotide specific oligonucleotide. The first strand synthesis reaction can then be used as a template for PCR amplification of the desired 5′ end using a primer specific to the ligated RNA oligonucleotide and a primer specific to the known sequence of the apoptosis related of interest. The resultant product is then sequenced and analyzed to confirm that the 5′ end sequence belongs to the relevant sequence of interest.

### Example 5

HCLI Translocation and Chloride-Effulx Assays

The activity of HCLI or homologues thereof, can be measured using any assay suitable for the measurement of the activity of an ion channel protein, as commonly known in the art.

To prove that HCLI is redistributed within the cell in response to changes in intracellular Cl⁻ concentration (like that of its closest homolog, Parchorin), HCLI is tagged with GFP (green fluorescence protein) and transiently/stably transfected in a variety of standard cultured cells. Under normal growth conditions, HCLI is expected to be diffusely distributed throughout the cytosol. Moving the transiently transfected cells into growth media low in Cl⁻ concentration would cause HCLI to translocate to the plasma membrane. Returning the cells to normal Cl⁻ conditions would re-distribute HCLI back to the cytosol. As HCLI is tagged with GFP (i.e. a GFP-HCLI fusion protein), the intracellular localization of GFP-HCLI can be monitored using fluorescence or confocal microscopy of the transfected cells.

To detect HCLI chloride ion efflux function, the chloride-sensitive fluorescent dye, 6-methoxy-N-[3-sulfopropyl] quinolinium (SPQ) is used (Chao, A. et al., 1989, Biophys. J., 56:1071-1081). In brief, cells transfected with HCLI-GFP expression vectors are grown on glass coverslips and are incubated with 25 mM SPQ in loading buffer (101 mM NaCl, 5 mM Kcl, 2mM CaCl₂, 5 mM Hepes, pH 7.4, 29 mM sodium gluconate) diluted 1:1 with water for 4 minutes at room temperature. Cells are then washed for 1 minute with loading buffer before transfer to a perfusion chamber maintained at 37° C. and viewed with a ×20 microscope objective. Fluorescence is excited at 355 nm and detected at 450 nm with an interference filter (435±20 nm).

Time course of SPQ fluorescence intensity is monitored using, for example, an Argus-50 system (Hamamatsu Photonics). For each measurement, a field is selected, including several GFP-positive cells (indicating HCLI has been transfected in these cells) adjacent to GFP-negative cells (indicating no HCLI has been transfected), to minimize fluctuations in the response to the changes of the outer medium. Each measurement is performed at an acquisition rate of 30 s per point, and the relative intensity of each point is normalized by the initial intensity of fluorescence. The initial loss of fluorescence due to the passive loss of SPQ from the cell is monitored for 10 minutes of perfusion with normal Cl⁻ solution. The perfusate is then switched to Cl⁻-free solution (101 mM sodium gluconate, 5 mM potassium gluconate, 2 mM calcium acetate, 2 mM MgSO₄, 50 mM mannitol, 5 mM Hepes/Tris, pH 7.4), whereupon the greater efflux of Cl⁻ (reduction of intracellular Cl⁻ concentration) is observed for HCLI transfected cells as an increase in fluorescence.

As the passive loss of SPQ from cells is fast (t_{1/2}˜10 minutes) during perfusion at 37° C., each record of the time course is processed as follows. For the first 10 minutes in normal Cl⁻ solution, an exponential curve is constructed by regression analysis to estimate the diffusional loss of SPQ from the cell. The projection of this regression curve is used to correct the relative fluorescence intensity for all subsequent time points in each experiment. These corrected time course data is used to calculate the Cl⁻ efflux rate. As about 2 minutes are required for replacement of the perfusate, the efflux rate is calculated by a linear fitting of the fluorescence values (slope) between 2 and 7 minutes after solution change.

### Example 6

Expression Profiling of the HCLI Polypeptide

The same PCR primer pair (SEQ ID NOS: 13-14) that was used to identify HCLI cDNA clones was used to measure the steady state levels of mRNA by quantitative PCR.

Briefly, first strand cDNA was made from commercially available poly A+ mRNA (Clontech) and subjected to real time quantitative PCR using a PE 5700 instrument (Applied Biosystems, Foster City, Calif.) which detects the amount of DNA amplified during each cycle by the fluorescent output of SYBR green, a DNA binding dye specific for double strands. The specificity of the primer pair for its target is verified by performing a thermal denaturation profile at the end of the run which provides an indication of the number of different DNA sequences present by determining melting Tm. In the case of the HCLI primer pair (SEQ ID NOS: 13-14), only one DNA fragment was detected having a homopolynucleotideous melting point. Contributions of contaminating genomic DNA to the assessment of tissue abundance is controlled for by performing the PCR with first strand cDNA made with and without reverse transcriptase. In all cases, the contribution of material amplified in the no reverse transcriptase controls was negligible. Small variations in the amount of cDNA used in each tube was determined by performing a parallel experiment using a primer pair for a polynucleotide expressed in equal amounts in all tissues, cyclophilin. These data were used to normalize the data obtained with the HCLI primer pair. The PCR data was converted into a relative assessment of the difference in transcript abundance amongst the tissues tested and the data are presented in bar graph form (see FIG. 8).

Detailed Methods

A. RNA Clean-Up

Five μg of poly A+mRNA (Clontech) was diluted to a volume of 77 μl with DEPC H₂O. A reaction mix cocktail was prepared for samples, calculating an amount sufficient for one extra reaction in case of pipetting errors. The components and amounts of the mix, per reaction, were: 10 μl of 10×PCR Buffer, 8 μl of 25 mM MgCl₂, 2.5 μl of RNase-OUT 40 U/μl, 2.5 μl of RNase-Free DNase. Thus, 23 μl of the cocktail mix were added to each sample, and the sample was incubated at room temperature for 15 minutes. One μl of 250 mM EDTA was then added, and the reaction was incubated at 65° C. for 15 minutes. Afterwards the reactions was placed on ice.

One hundred μl of a phenol:chloroform: isoamyl mixture was then added, and the sample was vortexed for 1 minute. The sample was then spun at 12,000 rpm for 2 minutes. Ninety to ninety-five μl of the top aqueous phase was removed and transferred to a new tube.

To precipitate the RNA, 1 μl of glycogen (20 μg/μl), 15 μl of 2M sodium acetate and 290 μl of 100% ethanol was added to the recovered aqueous phase. This mixture was incubated at −20° C. for 1 hour, and spun for 30 minutes at 4° C. to pellet the RNA. The pellet was washed in 500 μl of 70% ethanol, and air-dried. The pellet was resuspended in 22 μl of RNase-free water. (All of the above components were RNase-free, i.e., DEPC-treated).

B. First Strand cDNA Synthesis

The resuspended RNA was split equally into 2 tubes (one tube as the test sample, one tube as the negative control (i.e., no reverse-transcriptase is added to the negative control). To each tube, 1 μl of oligo(dT) primer was added. To anneal the primer to the RNA, the tubes were incubated at 70° C. for 10 minutes and the reaction was allowed to cool to room temperature. Reactions were subsequently placed on ice.

A cocktail mix was prepared, where enough mix is prepared for one extra reaction for pipetting errors. The mixture contained, per reaction, 2 μl of 10×PCR buffer, 2 μl of 25 mM MgCl₂, 1 μl of 10 mM dNTP mix, and 0.1 M DTT. Seven μl of the cocktail mix was added to each sample and incubated at 42° C. for 5 minutes. One μl of SuperScriptII reverse-transcriptase was added to test samples and one μl of DEPC water was added to negative-control samples. All of the samples were then incubated at 42° C. for 50 minutes. Reactions were terminated by incubating the samples at 70° C. for 15 minutes in order to inactivate the reverse-transcriptase. Samples were placed on ice. One μl of RNase H was added and samples were incubated at 37° C. for 20 minutes. Seventy-nine μl of water was then added in order to adjust the concentration of cDNA to 2.5 ng/μl (assuming 100% conversion of RNA to cDNA).

C. Quantitative PCR

All samples were run in triplicate, so sample tubes needed 3.5× reaction's worth of reaction mix. The reaction mix was composed of 2× SybrGreen master mix (25 μl per reaction), water (23.5 μl per reaction), primer mix (10 μM of each primer, for a total of 0.5 μl per reaction), and cDNA (2.5 ng/μl; 1 μl per reaction). First, the reaction mix was made minus the cDNA for enough reactions as determined above. 171.5 μl of the reaction mixture was added to each sample tube. Then 3.5 μl of cDNA was added to each sample tube. The mixture was gently mixed and spun down. Three 50 μl aliquots of each sample were placed on optical plates for quantitative PCR.

The PE 5700 instrument (Applied Biosystems, Foster City, Calif.), was set-up. Primer and sample set-up option was entered. The optical plate file was saved. The default program was run (the dissociation protocol box was checked). Immediately after the run, the file was saved again (before analyzing the data).

The data was then analyzed. The threshold was set in log view to intersect the linear region of amplification. The background was set in linear view to 2-3 cycles before the amplification curve appears. The analyze option was clicked. The mean values for the test samples were calculated. The values were normalized to cyclophilin: d_{Ct}=sample mean—cyclophilin mean. The dd_{Ct} was determined by subtracting individual d_{Cts} from the highest value of d_{Ct} in the list. The relative abundance was determined by the formula 2\hat{ }dd_{Ct}.

FIG. 8 illustrates the relative abundance of the chloride intracellular channel-related protein HCLI. HCLI is highly expressed in stomach, lung, and heart. Low levels of HCLI expression was detected in other tissues as shown.

### Example 7

Method of Assessing the Expression Profile of the Novel Intracellular Chloride Ion Channe; Poylpeptides of the Present Invetion using Expanded mRNA Tissue and Cell Sources

Total RNA from tissues was isolated using the TriZol protocol (Invitrogen) and quantified by determining its absorbance at 260 nM. An assessment of the 18 s and 28 s ribosomal RNA bands was made by denaturing gel electrophoresis to determine RNA integrity.

The specific sequence to be measured was aligned with related genes found in GenBank to identity regions of significant sequence divergence to maximize primer and probe specificity. Gene-specific primers and probes were designed using the ABI primer express software to amplify small amplicons (150 base pairs or less) to maximize the likelihood that the primers function at 100% efficiency. All primer/probe sequences were searched against Public Genbank databases to ensure target specificity. Primers and probes were obtained from ABI.

For HCLI, the primer probe sequences were as follows

| | |
|---|---|
| Forward Primer | |
| 5′-TTCTGGCTGATCTGTGGCTTT-3′ | (SEQ ID NO:18) |
| Reverse Primer | |
| 5′-GCACTCAGCCCACACACAAA-3′ | (SEQ ID NO:19) |
| TaqMan Probe | |
| 5′-CCTCCACCATCCCTAACCAACCTCTCAT-3′ | (SEQ ID NO:20) |

DNA Contamination

To access the level of contaminating genomic DNA in the RNA, the RNA was divided into 2 aliquots and one half was treated with Rnase-free Dnase (Invitrogen). Samples from both the Dnase-treated and non-treated were then subjected to reverse transcription reactions with (RT+) and without (RT−) the presence of reverse transcriptase. TaqMan assays were carried out with gene-specific primers (see above) and the contribution of genomic DNA to the signal detected was evaluated by comparing the threshold cycles obtained with the RT+/RT− non-Dnase treated RNA to that on the RT+/RT− Dnase treated RNA. The amount of signal contributed by genomic DNA in the Dnased RT− RNA must be less that 10% of that obtained with Dnased RT+RNA. If not the RNA was not used in actual experiments.

Reverse Transcription Reaction and Sequence Detection

100 ng of Dnase-treated total RNA was annealed to 2.5 μM of the respective gene-specific reverse primer in the presence of 5.5 mM Magnesium Chloride by heating the sample to 72° C. for 2 min and then cooling to 55° C. for 30 min. 1.25 U/μl of MuLv reverse transcriptase and 500 μM of each dNTP was added to the reaction and the tube was incubated at 37° C. for 30 min. The sample was then heated to 90° C. for 5 min to denature enzyme.

Quantitative sequence detection was carried out on an ABI PRISM 7700 by adding to the reverse transcribed reaction 2.5 μM forward and reverse primers, 2.0 μM of the TaqMan probe, 500 μM of each dNTP, buffer and 5U AmpliTaq GoId™. The PCR reaction was then held at 94° C. for 12 min, followed by 40 cycles of 94° C. for 15 sec and 60° C. for 30 sec.

Data Handling

The threshold cycle (Ct) of the lowest expressing tissue (the highest Ct value) was used as the baseline of expression and all other tissues were expressed as the relative abundance to that tissue by calculating the difference in Ct value between the baseline and the other tissues and using it as the exponent in 2^{(ΔCt)}

The expanded expression profile of the HCLI polypeptide is provided in FIGS. 9 and 10 and described elsewhere herein.

### Example 8

Ligand Binding Assay for High Throughput Screening of HCLI Modulators

Cell lines that over-express the HCLI coding region described herein (SEQ ID NO: 1), or a biologically active fragment or truncated portion thereof, or a chimeric or fusion protein, are used in binding assays to identify and screen for pharmacologically active molecules that block HCLI activity.

A radiolabeled binding assay using a radiolabeled ligand is employed (Hill, R. J., 1995, Mol. Pharm., 48:98; and Deutsch, C. et al., 1991, J. Biol. Chem., 266:3668). As HCLI may translocate to the plasma membrane and in low Cl⁻ media conditions and as HCLI may be localized to intracellular membranes, membrane preparations of cell lines that over-express HCLI are made by homogenizing the cells using a Polytron for 25 seconds at 13,000 rpm and centrifuged at 100×g for 2 minutes. The pellet is resuspended in 1 ml of assay buffer (5 mM NaCl, 5 mM KCl, 10 mM HEPES, 6 mM glucose, pH 8.4) and diluted to 50 μg/ml. Alternatively, if HCLI is localized to the cytosol in over-expressed cells, then whole cell lysates may be prepared.

To each of the wells of a 96-well microtiter plate, 130 μl of asssay buffer is added, along with 20 μl of test compound or drug (the test compound or drug may be a small molecule, peptide, analog, or mimetic agent), control assay buffer, non-specific unlabeled ligand (10 nM), 50 μl of membranes (or whole cell lysate) from cells over-expressing HCLI at 50 μg/ml, and 50 μl of radioligand (25 pM; NEN, 2200 Ci/mmol). The plates are incubated for 20 minutes at 21° C. with mixing. Bound radiolabeled ligand is separated from free radiolabeled ligand in solution by filtration over pre-soaked GF/C Unifliters (Packard Instruments) and washed rapidly in ice-cold wash buffer. Upon drying, scintillation fluid is added and the filter plates are scintillation counted. Data from saturation experiments are subjected to Scatchard analysis and linear regression (Deutsch, C. et al., 1991, J. Biol. Chem., 266:3668). Compounds that compete with the radiolabeled ligand for binding the novel HCLI chloride channel are identified via the reduction in specific counts. Alternatively, a scintillation proximity assay (SPA) can be used so as to eliminate the need for filters. SPA is easily adapted for high throughput screening assays (Hoffman, R. et al., 1992, Anal. Biochem., 29:370; Kienuis, C. et al., 1992, J. Recept. Res., 12:389).

### Example 9

Method of Creating N- and C-Terminal Deletion Mutants Corresponding to the HCLI Polypeptide of the Present Invention

As described elsewhere herein, the present invention encompasses the creation of N- and C-terminal deletion mutants, in addition to any combination of N- and C-terminal deletions thereof, corresponding to the HCLI polypeptide of the present invention. A number of methods are available to one skilled in the art for creating such mutants. Such methods may include a combination of PCR amplification and polynucleotide cloning methodology. Although one of skill in the art of molecular biology, through the use of the teachings provided or referenced herein, and/or otherwise known in the art as standard methods, could readily create each deletion mutant of the present invention, exemplary methods are described below.

Briefly, using the isolated cDNA clone encoding the full-length HCLI polypeptide sequence (as described in Example 3, for example), appropriate primers of about 15-25 nucleotides derived from the desired 5′ and 3′ positions of SEQ ID NO: 1 may be designed to PCR amplify, and subsequently clone, the intended N- and/or C-terminal deletion mutant. Such primers could comprise, for example, an inititation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, kozak sequences, or other sequences discussed and/or referenced herein.

Representative PCR amplification conditions are provided below, although the skilled artisan would appreciate that other conditions may be required for efficient amplification. A 100 ul PCR reaction mixture may be prepared using 10 ng of the template DNA (cDNA clone of HCLI), 200 uM 4dNTPs, 1 uM primers, 0.25U Taq DNA polymerase (PE), and standard Taq DNA polymerase buffer. Typical PCR cycling condition are as follows: 20-25 cycles of: (45 sec, 93 degrees; 2 min, 50 degrees; 2 min, 72 degrees) and 1 cycle of: (10 min, 72 degrees). After the final extension step of PCR, 5U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). . The skilled artisan would appreciate that other plasmids could be equally substituted, and may be desirable in certain circumstances. The digested fragment and vector are then ligated using a DNA ligase, and then used to transform competent E.coli cells using methods provided herein and/or otherwise known in the art.

The 5′ primer sequence for amplifying any additional N-terminal deletion mutants may be determined by reference to the following formula: (S+(X*3)) to ((S+(X*3))+25), wherein ‘S’ is equal to the nucleotide position of the initiating start codon of the HCLI polynucleotide (SEQ ID NO: 1), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer corresponding to sense strand of SEQ ID NO: 1. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozak sequences, etc.).

The 3′ primer sequence for amplifying any additional N-terminal deletion mutants may be determined by reference to the following formula: (S+(X*3)) to ((S+(X*3))−25), wherein ‘S’ is equal to the nucleotide position of the initiating start codon of the HCLI polynucleotide (SEQ ID NO: 1), and ‘X’ is equal to the most C-terminal amino acid of the intended N-terminal deletion mutant. The first term will provide the start 5′ nucleotide position of the 3′ primer, while the second term will provide the end 3′ nucleotide position of the 3′ primer corresponding to the anti-sense strand of SEQ ID NO: 1. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same polynucleotidal formulas provided above may be used in identifying the 5′ and 3′ primer sequences for amplifying any C-terminal deletion mutant of the present invention. Moreover, the same polynucleotide formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

The above-mentioned aspects, objects, relations, provisions, embodiments, and examples of the invention that are provided for the HCLI polynucleotide (SEQ ID NO: 1) and its encoded polypeptide (SEQ ID NO: 2) are also construed and thus provided for the HCLI variant polynucleotide (SEQ ID NO: 3) and its encoded polypeptide (SEQ ID NO: 4).

The contents of all patents, patent applications, published PCT applications and articles, books, references, reference manuals, abstracts and internet websites cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the sequence listing submitted herewith and the corresponding computer readable for rn are both incorporated herein by reference in their entireties.

What is claimed is:

1. An isolated nucleic acid molecule comprising a polynucleotide having a nucleotide sequence selected from the group consisting of:
(a) a polynucleotide fragment of SEQ ID NO: 1 or a polynucleotide fragment of the cDNA sequence included in ATCC Deposit No: PTA-4803, which is hybridizable to SEQ ID NO: 1;
(b) a polynucleotide encoding a polypeptide fragment of SEQ ID NO: 2 or a polypeptide fragment encoded by the cDNA sequence included in ATCC Deposit No: PTA-4803, which is hybridizable to SEQ ID NO: 1;
(c) a polynucleotide encoding a polypeptide domain of SEQ ID NO: 2 or a polypeptide domain encoded by the cDNA sequence included in ATCC Deposit No: PTA-4803, which is hybridizable to SEQ ID NO: 1;
(d) a polynucleotide encoding a polypeptide epitope of SEQ ID NO: 2 or a polypeptide epitope encoded by the cDNA sequence included in ATCC Deposit No: PTA-4803, which is hybridizable to SEQ ID NO: 1;
(e) a polynucleotide encoding a polypeptide of SEQ ID NO: 2 or the cDNA sequence included in ATCC Deposit No: PTA-4803, which is hybridizable to SEQ ID NO: 1, having biological activity;
(f) an isolated polynucleotide comprising nucleotides 4 to 1887 of SEQ ID NO: 1, wherein said nucleotides encode a polypeptide corresponding to amino acids 2 to 629 of SEQ ID NO: 2 of SEQ ID NO: 2 minus the start methionine;
(g) an isolated polynucleotide comprising nucleotides 1 to 1887 of SEQ ID NO: 1, wherein said nucleotides encode a polypeptide corresponding to amino acids 1 to 629 of SEQ ID NO: 2 including the start methionine;
(h) a polynucleotide which represents the complimentary sequence (antisense) of SEQ ID NO: 1;
(i) a polynucleotide fragment of SEQ ID NO: 3 or a polynucleotide fragment of the cDNA sequence included in ATCC Deposit No: PTA-4803, which is hybridizable to SEQ ID NO: 3;
(j) a polynucleotide encoding a polypeptide fragment of SEQ ID NO: 4 or a polypeptide fragment encoded by the cDNA sequence included in ATCC Deposit No: PTA-4803, which is hybridizable to SEQ ID NO: 3;
(k) a polynucleotide encoding a polypeptide domain of SEQ ID NO: 4 or a polypeptide domain encoded by the cDNA sequence included in ATCC Deposit No: PTA-4803, which is hybridizable to SEQ ID NO: 3;
(I) a polynucleotide encoding a polypeptide epitope of SEQ ID NO: 4 or a polypeptide epitope encoded by the cDNA sequence included in ATCC Deposit No: PTA-4803, which is hybridizable to SEQ ID NO: 3;
(m) a polynucleotide encoding a polypeptide of SEQ ID NO: 4 or the cDNA sequence included in ATCC Deposit No: PTA-4803, which is hybridizable to SEQ ID NO: 3, having biological activity;
(n) an isolated polynucleotide comprising nucleotides 2 to 1153 of SEQ ID NO: 3, wherein said nucleotides encode a polypeptide corresponding to amino acids I to 384 of SEQ ID NO: 4;
(o) a polynucleotide which represents the complimentary sequence (antisense) of SEQ ID NO: 3;
(p) a polynucleotide fragment of SEQ ID NO: 16 or a polynucleotide fragment of the cDNA sequence included in ATCC Deposit No: PTA-4803, which is hybridizable to SEQ ID NO: 16;
(q) a polynucleotide encoding a polypeptide fragment of SEQ ID NO: 17 or a polypeptide fragment encoded by the cDNA sequence included in ATCC Deposit No: PTA-4803, which is hybridizable to SEQ ID NO: 16;
(r) a polynucleotide encoding a polypeptide domain of SEQ ID NO: 17 or a polypeptide domain encoded by the cDNA sequence included in ATCC Deposit No: PTA-4803, which is hybridizable to SEQ ID NO: 16;
(s) a polynucleotide encoding a polypeptide epitope of SEQ ID NO: 17 or a polypeptide epitope encoded by the cDNA sequence included in ATCC Deposit No: PTA-4803, which is hybridizable to SEQ ID NO: 16;
(t) a polynucleotide encoding a polypeptide of SEQ ID NO: 17 or the cDNA sequence included in ATCC Deposit No: PTA-4803, which is hybridizable to SEQ ID NO: 16, having biological activity;
(u) an isolated polynucleotide comprising nucleotides 4 to 2058 of SEQ ID NO: 16, wherein said nucleotides encode a polypeptide corresponding to amino acids 2 to 686 of SEQ ID NO: 17 of SEQ ID NO: 17 minus the start methionine;
(v) an isolated polynucleotide comprising nucleotides 1 to 2058 of SEQ ID NO: 16, wherein said nucleotides encode a polypeptide corresponding to amino acids 1 to 686 of SEQ ID NO: 17 including the start methionine;
(w) a polynucleotide which represents the complimentary sequence (antisense) of SEQ ID NO: 16; and
(x) a polynucleotide capable of hybridizing under stringent conditions to any one of the polynucleotides specified in (a)-(w), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment consists of a nucleotide sequence encoding a human intracellular chloride ion channel.

3. A recombinant vector comprising the isolated nucleic acid molecule of claim 1.

4. A recombinant host cell comprising the vector sequences of claim 3.

5. An isolated polypeptide comprising an amino acid sequence selected from the group consisting of:
(a) a polypeptide fragment of SEQ ID NO: 2 or the encoded sequence included in ATCC Deposit No: PTA-4803;
(b) a polypeptide fragment of SEQ ID NO: 2 or the encoded sequence included in ATCC Deposit No: PTA-4803, having ion flux activity;
(c) a polypeptide domain of SEQ ID NO: 2 or the encoded sequence included in ATCC Deposit No: PTA-4803;
(d) a polypeptide epitope of SEQ ID NO: 2 or the encoded sequence included in ATCC Deposit No: PTA-4803;
(e) a full length protein of SEQ ID NO: 2 or the encoded sequence included in ATCC Deposit No: PTA-4803;
(f) a polypeptide comprising amino acids 2 to 629 of SEQ ID NO: 2, wherein said amino acids 2 to 629 comprising a polypeptide of SEQ ID NO: 2 minus the start methionine;
(g) a polypeptide comprising amino acids 1 to 629 of SEQ ID NO: 2;
(h) a polypeptide fragment of SEQ ID NO: 4 or the encoded sequence included in ATCC Deposit No: PTA-4803;
(i) a polypeptide fragment of SEQ ID NO: 4 or the encoded sequence included in ATCC Deposit No: PTA-4803, having ion flux activity;
(j) a polypeptide domain of SEQ ID NO: 4 or the encoded sequence included in ATCC Deposit No: PTA-4803;
(k) a polypeptide epitope of SEQ ID NO: 4 or the encoded sequence included in ATCC Deposit No: PTA-4803;
(I) a full length protein of SEQ ID NO: 4 or the encoded sequence included in ATCC Deposit No: PTA-4803;a full length protein of SEQ ID NO: 4;
(m) a polypeptide comprising amino acids 1 to 384 of SEQ ID NO: 4;
(n) a polypeptide fragment of SEQ ID NO: 17 or the encoded sequence included in ATCC Deposit No: PTA-4803;
(o) a polypeptide fragment of SEQ ID NO: 17 or the encoded sequence included in ATCC Deposit No: PTA-4803, having ion flux activity;
(p) a polypeptide domain of SEQ ID NO: 17 or the encoded sequence included in ATCC Deposit No: PTA-4803;
(q) a polypeptide epitope of SEQ ID NO: 17 or the encoded sequence included in ATCC Deposit No: PTA-4803;
(r) a full length protein of SEQ ID NO: 17 or the encoded sequence included in ATCC Deposit No: PTA-4803;a full length protein of SEQ ID NO: 17;
(s) a polypeptide comprising amino acids 2 to 686 of SEQ ID NO: 2, wherein said amino acids 2 to 686 comprising a polypeptide of SEQ ID NO: 2 minus the start methionine; and
(t) a polypeptide comprising amino acids 1 to 686 of SEQ ID NO: 17.

6. The isolated polypeptide of claim 5, wherein the full length protein comprises sequential amino acid deletions from either the C-terminus or the N-terminus.

7. An isolated antibody that binds specifically to the isolated polypeptide of claim 5.

8. A recombinant host cell that expresses the isolated polypeptide of claim 5.

9. A method of making an isolated polypeptide comprising:
(a) culturing the recombinant host cell of claim 8 under conditions such that said polypeptide is expressed; and
(b) recovering said polypeptide.

10. The polypeptide produced by claim 9.

11. A method for preventing, treating, or ameliorating a medical condition, comprising the step of administering to a mammalian subject a therapeutically effective amount of the polypeptide of claim 5, or a modulator thereof.

12. A method of diagnosing a pathological condition or a susceptibility to a pathological condition in a subject comprising:
(a) determining the presence or absence of a mutation in the polynucleotide of claim 1; and
(b) diagnosing a pathological condition or a susceptibility to a pathological condition based on the presence or absence of said mutation.

13. A method of diagnosing a pathological condition or a susceptibility to a pathological condition in a subject comprising:
(a) determining the presence or amount of expression of the polypeptide of claim 5 in a biological sample; and
(b) diagnosing a pathological condition or a susceptibility to a pathological condition based on the presence or amount of expression of the polypeptide.

14. An isolated nucleic acid molecule consisting of a polynucleotide having a nucleotide sequence selected from the group consisting of:
(a) a polynucleotide encoding a polypeptide of SEQ ID NO: 2;
(b) an isolated polynucleotide consisting of nucleotides 4 to 1887 of SEQ ID NO: 1, wherein said nucleotides encode a polypeptide corresponding to amino acids 2 to 629 of SEQ ID NO: 2 minus the start methionine;
(c) an isolated polynucleotide consisting of nucleotides 1 to 1887 of SEQ ID NO: 1, wherein said nucleotides encode a polypeptide corresponding to amino acids 1 to 629 of SEQ ID NO: 2 including the start methionine;
(d) a polynucleotide encoding the HCLI polypeptide encoded by the cDNA clone contained in ATCC Deposit No. PTA-4803;
(e) a polynucleotide which represents the complimentary sequence (antisense) of SEQ ID NO: 1;
(f) a polynucleotide encoding a polypeptide of SEQ ID NO: 4;
(g) an isolated polynucleotide consisting of nucleotides 2 to 1153 of SEQ ID NO: 29, wherein said nucleotides encode a polypeptide corresponding to amino acids 1 to 384 of SEQ ID NO: 4;
(h) a polynucleotide encoding the HCLI.v1 variant polypeptide encoded by the cDNA clone contained in ATCC Deposit No. PTA-4803;
(i) a polynucleotide which represents the complimentary sequence (antisense) of SEQ ID NO: 3;
(j) a polynucleotide encoding a polypeptide of SEQ ID NO: 17;
(k) an isolated polynucleotide consisting of nucleotides 4 to 2058 of SEQ ID NO: 16, wherein said nucleotides encode a polypeptide corresponding to amino acids 2 to 686 of SEQ ID NO: 17 minus the start methionine;
(I) an isolated polynucleotide consisting of nucleotides 1 to 2058 of SEQ ID NO: 16, wherein said nucleotides encode a polypeptide corresponding to amino acids 1 to 686 of SEQ ID NO: 17 including the start methionine;
(m) a polynucleotide encoding the HCLI polypeptide encoded by the cDNA clone contained in ATCC Deposit No. PTA-4803; and
(n) a polynucleotide which represents the complimentary sequence (antisense) of SEQ ID NO: 16.

15. The isolated nucleic acid molecule of claim 14, wherein the polynucleotide comprises a nucleotide sequence encoding a human intracellular chloride ion channel.

16. A recombinant vector comprising the isolated nucleic acid molecule of claim 15.

17. A recombinant host cell comprising the recombinant vector of claim 16.

18. An isolated polypeptide consisting of an amino acid sequence selected from the group consisting of:
(a) a polypeptide fragment of SEQ ID NO: 2 having ion flux activity;
(b) a polypeptide domain of SEQ ID NO: 2 having ion flux activity;
(c) a full length protein of SEQ ID NO: 2;
(d) a polypeptide corresponding to amino acids 2 to 629 of SEQ ID NO: 2, wherein said amino acids 2 to 629 consisting of a polypeptide of SEQ ID NO: 2 minus the start methionine;
(e) a polypeptide corresponding to amino acids 1 to 629 of SEQ ID NO: 2;
(f) a polypeptide encoded by the cDNA contained in ATCC Deposit No. PTA-4803;
(g) a polypeptide corresponding to amino acids 1 to 384 of SEQ ID NO: 4;
(h) a polypeptide fragment of SEQ ID NO: 17 having ion flux activity;
(i) a polypeptide domain of SEQ ID NO: 17 having ion flux activity;
(j) a full length protein of SEQ ID NO: 17;
(k) a polypeptide corresponding to amino acids 2 to 686 of SEQ ID NO: 17, wherein said amino acids 2 to 686 consisting of a polypeptide of SEQ ID NO: 17 minus the start methionine; and
(I) a polypeptide encoded by the cDNA contained in ATCC Deposit No. PTA-4803.

19. The method of diagnosing a pathological condition of claim 15 wherein the condition is a member of the group consisting of: a disorder related to aberrant chloride channel function; a disorder related to aberrant chloride regulation; disorders involving aberrant chloride/ion homeostasis; disorders involving aberrant chloride/ion transport; disorders involving aberrant chloride/ion homeostasis in the choroid plexus; choroid plexus disorders; hyponatremia; hypernatremia; disorders involving aberrant chloride/ion homeostasis in the lung; cystic fibrosis; disorders involving aberrant chloride/ion homeostasis in the liver; cirrhosis; disorders involving aberrant chloride/ion homeostasis in the gall bladder; cholecystitis; neuroprotection disorders; disorders involving aberrant influx of drugs in the central nervous system; disorders involving aberrant efflux of drugs in the central nervous system; disorders involving aberrant cerebral spinal fluid synthesis; disorders involving aberrant cerebral spinal fluid volume; disorders involving aberrant cerebral spinal fluid composition; disorders involving aberrant glucose levels in cerebral spinal fluid; disorders involving aberrant amino acid levels in cerebral spinal fluid; disorders involving aberrant transthyretin expression; disorders involving aberrant transthyretin regulation; disorders involving aberrant thyroid hormone transport in the choroid plexus; disorders involving aberrant thyroid hormone transport in the central nervous system; disorders involving aberrant central nervous system inflammation; disorders involving aberrant central nervous system development; disorders involving aberrant central nervous system function; choroid plexus tumors; choroid plexus papillomas; hepatic disorders; cirrhosis; disorders involving aberrant inflammation of the liver; cardiovascular disorders; congestive heart failure; cysts; and vascular disorders.

20. The method for preventing, treating, or ameliorating a medical condition of claim 11, wherein the medical condition is selected from the group consisting of: a disorder related to aberrant chloride channel function; a disorder related to aberrant chloride regulation; disorders involving aberrant chloride/ion homeostasis; disorders involving aberrant chloride/ion transport; disorders involving aberrant chloride/ion homeostasis in the choroid plexus; choroid plexus disorders; hyponatremia; hypernatremia; disorders involving aberrant chloride/ion homeostasis in the lung; cystic fibrosis; disorders involving aberrant chloride/ion homeostasis in the liver; cirrhosis; disorders involving aberrant chloride/ion homeostasis in the gall bladder; cholecystitis; neuroprotection disorders; disorders involving aberrant influx of drugs in the central nervous system; disorders involving aberrant efflux of drugs in the central nervous system; disorders involving aberrant cerebral spinal fluid synthesis; disorders involving aberrant cerebral spinal fluid volume; disorders involving aberrant cerebral spinal fluid composition; disorders involving aberrant glucose levels in cerebral spinal fluid; disorders involving aberrant amino acid levels in cerebral spinal fluid; disorders involving aberrant transthyretin expression; disorders involving aberrant transthyretin regulation; disorders involving aberrant thyroid hormone transport in the choroid plexus; disorders involving aberrant thyroid hormone transport in the central nervous system; disorders involving aberrant central nervous system inflammation; disorders involving aberrant central nervous system development; disorders involving aberrant central nervous system function; choroid plexus tumors; choroid plexus papillomas; hepatic disorders; cirrhosis; disorders involving aberrant inflammation of the liver; cardiovascular disorders; congestive heart failure; cysts; and vascular disorders.


SEQUENCE LISTING (20 sequences)

SEQ ID NO: 1 - DNA, 3641 bp, Homo sapiens
  CDS (1)..(1887)
atggccgaggccgcggagccggagggggttgccccgggtccccaggggccgccggaggtccccgcgcctctggctgagagacccggagagccaggagccgcgggcggggaggcagaagggccggaggggagcgagggcgcagaggaggcgccgaggggcgccgccgctgtgaaggaggcaggaggcggcgggccagacaggggcccggaggccgaggcgcggggcacgaggggggcgcacggcgagactgaggccgaggagggagccccggagggtgccgaggtgccccaaggaggggaggagacaagcggcgcgcagcaggtggagggggcgagcccgggacgcggcgcgcagggcgagccccgcggggaggctcagagggagcccgaggactctgcggcccccgagaggcaggaggaggcggagcagaggcctgaggtcccggaaggtagcgcgtccggggaggcgggggacagcgtagacgcggagggcccgctgggggacaacatagaagcggagggcccggcgggcgacagcgtagaggcggagggccgggtgggggacagcgtagacgcggaagaggcgggggacccggcgggggacggcgtagaagcgggggtcccggcgggggacagcgtagaagccgaaggcccggcgggggacagcatggacgccgagggtccggcaggaagggcgcgccgggtctcgggtgagccgcagcaatcgggggacggcagcctctcgccccaggccgaggcaattgaggtcgcagccggggagagtgcggggcgcagccccggtgagctcgcctgggacgcagcggaggaggcggaggtcccgggggtaaaggggtccgaagaagcggcccccggggacgcaagggcagacgctggcgaggacagggtaggggatgggccacagcaggagccgggggaggacgaagagagacgagagcggagcccggaggggccaagggaggaggaagcagcggggggcgaagaggaatcccccgacagcagcccacatggggaggcctccaggggcgccgcggagcctgaggcccagctcagcaaccacctggccgaggagggccccgccgagggtagcggcgaggccgcgcgcgtgaacggccgcccggaggacggagaggcgtccgagccccgggccctggggcaggagcacgacatcaccctcttcgtcaaggctggttatgatggtgagagtatcggaaattgcccgttttctcagcgtctctttatgattctctggctgaaaggcgttatatttaatgtgaccacagtggacctgaaaaggaaacccgcagacctgcagaacctggctcccggaacaaaccctcctttcatgacttttgatggtgaagtcaagacggatgtgaataagatcgaggagttcttagaggagaaattagctcccccgaggtatcccaagctggggacccaacatcccgaatctaattccgcaggaaatgacgtgtttgccaaattctcagcctttataaaaaacacgaagaaggatgcaaatgagattcatgaaaagaacctgctgaaggccctgaggaagctggataattacttaaatagccctctgcctgatgaaatagatgcctacagcaccgaggatgtcactgtttctggaaggaagtttctggatggggacgagctgacgctggctgactgcaacctcttacccaagctccatattattaagattgtggccaagaagtacagagattttgaatttccttctgaaatgactggcatctggagatacttgaataatgcttatgctagagatgagttcacaaatacgtgtccagctgatcaagagattgaacacgcatattcagatgttgcaaaaagaatgaaatgaagctgggctgttttctgtcttatttctcagttgagtgagcaaggatacgaaaacagtgtgtttgaaaacaaattaggtttgggttcaattccttcaatttttaaaaaactggtctctgagagttttttaaatcattgagagcctgtttttcttctctaaaacattagtttaattttcttcaaaatgaaaatactgctttgtaattacaaaatgagacacacctatcttgatattttaaagcaatatcagagggtgtaaagaaggacattttaacaatcgccttcaattttactccacttaattaccgaaaacttactggagaacatgttccaaatcttcagtatcttgttctctctctctctctctctctctctctctctctatcacacacacacacacacacacacacacacaatttcattcatatatggtattgcattattttattttaaagcactggtgaggggacctcttggtgattcctggatgatcatacacagaggacttacaccatacaaaaatattgggcaccgcagtgccagagaagatgcttgaggttagattttaaggagtggggaattgtgaagcccacagatgcgcacgcaatgaccagcaggaaccggaagccctgggtcacccccactctgcctcatttctgcctccaggatgccactgcctctgcttccaggaaaggcaagggggagggtgcttatccgtgtctctggttccagcttcctgtctttgctccctcccctctcttagagactgtgccttcagcaggactcttaatatctgctgcaacttggagaacctccctgccctgaaatgtgaaccaagcacactttggttccttctccgggcggctctctctgagacccaaggctgcagtccctcagtgctggtggtatcgtgtggggatagcaatacttctctgcccttcattgtctcatctagacatcaagcagggaaaatccagggagattaaattcatggtgacaagtcctgggcagttctggtaaacggtccagttagggagggagtagaaactattgactcaaaagagtttctggctgatctgtggctttgcctccaccatccctaaccaacctctcatcacagctttgtgtgtgggctgagtgctggccttaaccctaggcgtggaagagaaaatgtgaggttgtttagatactcatcaggacctcacaggagctgagacttatcagccagaatgtgttcttcggacagtcgtacacatcttacagaaaaccctccttgtagagttggttgtggtatgtgtttgatgctataaagctcatttttaatgtgtacacctgctctagggacgattcgtttgaaagagagtaagatgcattaacagaaactatcctgggattaggtgaaaaatgtctagcaaaagaaacaaagtctttaatagagtggcctctttcgctcttcgattatgactgctgcagttttaccccagcagtcagctgtctttgtcaaccatacgtttttggagattgggtctagcacatgtcacccttgtccacgttgtttcacatctggttagggggcagattttaaaatgtagttttgtaatgttacatttaagcatgataaatgattagactaccagatgttactagtgctaactttgtattcttagacattaaaatgattgacataaactctttgtgccttgaaaatgaaacaaattataaaaatgtttaaatggaaaaaaaaaaaaaaag

SEQ ID NO: 2 - protein, 629 aa, Homo sapiens
MAEAAEPEGVAPGPQGPPEVPAPLAERPGEPGAAGGEAEGPEGSEGAEEAPRGAAAVKEAGGGGPDRGPEAEARGTRGAHGETEAEEGAPEGAEVPQGGEETSGAQQVEGASPGRGAQGEPRGEAQREPEDSAAPERQEEAEQRPEVPEGSASGEAGDSVDAEGPLGDNIEAEGPAGDSVEAEGRVGDSVDAEEAGDPAGDGVEAGVPAGDSVEAEGPAGDSMDAEGPAGRARRVSGEPQQSGDGSLSPQAEAIEVAAGESAGRSPGELAWDAAEEAEVPGVKGSEEAAPGDARADAGEDRVGDGPQQEPGEDEERRERSPEGPREEEAAGGEEESPDSSPHGEASRGAAEPEAQLSNHLAEEGPAEGSGEAARVNGRPEDGEASEPRALGQEHDITLFVKAGYDGESIGNCPFSQRLFMILWLKGVIFNVTTVDLKRKPADLQNLAPGTNPPFMTFDGEVKTDVNKIEEFLEEKLAPPRYPKLGTQHPESNSAGNDVFAKFSAFIKNTKKDANEIHEKNLLKALRKLDNYLNSPLPDEIDAYSTEDVTVSGRKFLDGDELTLADCNLLPKLHIIKIVAKKYRDFEFPSEMTGIWRYLNNAYARDEFTNTCPADQEIEHAYSDVAKRMK

SEQ ID NO: 3 - DNA, 1441 bp, Homo sapiens
  CDS (2)..(1153)
ggcagacgctggcgaggacagggtaggggatgggccacagcaggagccgggggaggacgaagagagacgagagcggagcccggaggggccaagggaggaggaagcagcggggggcgaagaggaatcccccgacagcagcccacatggggaggcctccaggggcgccgcggagcctgaggcccagctcagcaaccacctggccgaggagggccccgccgagggtagcggcgaggccgcgcgcgtgaacggccgccgggaggacggagaggcgtccgagccccgggccctggggcaggagcacgacatcaccctcttcgtcaaggctggttatgatggtgagagtatcggaaattgcccgttttctcagcgtctctttatgattctctggctgaaaggcgttatatttaatgtgaccacagtggacctgaaaaggaaacccgcagacctgcagaacctggctcccggaacaaaccctcctttcatgacttttgatggtgaagtcaagacggatgtgaataagatcgaggagttcttagaggagaaattagctcccccgaggtatcccaagctggggacccaacatcccgaatctaattccgcaggaaatgacgtgtttgccaaattctcagcgtttataaaaaacacgaagaaggatgcaaatgagattcatgaaaagaacctgctgaaggccctgaggaagctggataattacttaaatagccctctgcctgatgaaatagatgcctacagcaccgaggatgtcactgtttctggaaggaagtttctggatggggacgagctgacgctggctgactgcaacctcttacccaagctccatattattaaggttcatcttccctcccgacacgtgtgccgagtacacgaacggggctttgttttattttgtttttacttgtgtttgggccagacatttaaacagtctgaaatgtggagacttggattaaaaacactagttctgttcggggcagccaagccccagctttcacacacacagaagatgggcttcgggtgttctcagaaagtgcctgggaacatggccccaccttctgcttctctctcagccttactcacacagcctaaagacaggttatgtgaaagcagcctgaggaatttcgtagattatacctggtgttagcaattgagaattagagatgaggctcgtagaatctggggctattcaaatgtgaacgcagtctactgttggtgattgtagggctccacatggccccttctagggatgacagaggagtgttctattagcttttaacagtgacccttggccaggtgcagtggctcatgcctgtaatcccagtgatttgggaggctgaggtgggaggattgcttgaggccaggagttcaagaccagccagggcaacatagcgagaccccatctatacaaaaaataaaaaaaaaaaaaaag

SEQ ID NO: 4 - protein, 384 aa, Homo sapiens
ADAGEDRVGDGPQQEPGEDEERRERSPEGPREEEAAGGEEESPDSSPHGEASRGAAEPEAQLSNHLAEEGPAEGSGEAARVNGRREDGEASEPRALGQEHDITLFVKAGYDGESIGNCPFSQRLFMILWLKGVIFNVTTVDLKRKPADLQNLAPGTNPPFMTFDGEVKTDVNKIEEFLEEKLAPPRYPKLGTQHPESNSAGNDVFAKFSAFIKNTKKDANEIHEKNLLKALRKLDNYLNSPLPDEIDAYSTEDVTVSGRKFLDGDELTLADCNLLPKLHIIKVHLPSRHVCRVHERGFVLFCFYLCLGQTFKQSEMWRLGLKTLVLFGAAKPQLSHTQKMGFGCSQKVPGNMAPPSASLSALLTQPKDRLCESSLRNFVDYTWC

SEQ ID NO: 5 - protein, 637 aa, Oryctolagus cuniculus
MAETAEPEGGAPSPQGPPEGSALLEERPGEPDPAGPEASEGAAKAPSGEGAGAAAKAGATEEASGGRDGEGAGEQAPDAGTESGGETPDAKGAQIEAEGAPEGTKAPQLGEEGSGGKQVEESGPDCELRGEAAREAEGQAAAPAAPGAQEEAVPGDSVDAEGSIDAGGSVDAAGSVDAGGSIDAGGSMDAGGSVDAGGSIDTGGSVDAAGSVDAGGSIDTGRNVDAGGSIDAGGSVDAGGSMDAEGPAGGAHGAGGEPQDLGAGSPQPRSEAVEVAAAENEGHSPGESVEDAAAEEAAGTREPEGSEDAAGEDGDQGRPQEETEQQAERQEPGPETQSEEEERPPDRSPDGEAAASTRAAQPEAELSNHLAAEEGGQRGEGPANGRGEDGEASEEGDPGQEHDITLFVKAGYDGESIGNCPFSQRLFMILWLKGVIFNVTTVDLKRKPADLQNLAPGTNPPFMTFDGDVKTDVNKIEEFLEEKLAPPRYPKLATQHPESNSAGNDVFAKFSAFIKNTKKDANEIYEKSLLKALKKLDAYLNSPLPDEVDAYSTEDVAVSGRKFLDGDDLTLADCNLLPKLHIIKIVAKKYRDFEFPPEMTGIWRYLNNAYARDEFINTCPADQEIEHAYSDVAKRMK

SEQ ID NO: 6 - protein, 437 aa, Bos taurus
MNDENYSTTIYNRVQTERVYEDSDPAENGGPLYDEVHEDVRREDNLYVNELENQEYDSVAVYPVGRQGRTSASLQPETGEYVLPDEPYSKAQDPHPGEPTADEDISLEELLSPTKDHQSDSEEPQASDPEEPQASDPEEPQGPDPEEPQENGNEMEADLPSPSSFTIQNSRAFSTREISPTSYSADDVSEGNESASASPEINLFVKAGIDGESIGNCPFSQRLFMILWLKGVVFNVTTVDLKRKPADLHNLAPGTHPPFLTFNGDVKTDVNKIEEFLEETLTPEKYPRLAAKHRESNTAGIDIFVKFSAYIKNTKQQSNAALERGLTKALKKLDDYLNTPLPEEIDADTRGDDEKGSRRKFLDGDELTLADCNLLPKLHVVKIVAKKYRNYDFPAEMTGLWRYLKNAYARDEFTNTCAADSEIELAYADVAKRLSRS

SEQ ID NO: 7 - protein, 253 aa, Homo sapiens [sequence omitted]

SEQ ID NO: 8 - protein, 243 aa, Homo sapiens
MSGLRPGTQVDPEIELFVKAGSDGESIGNCPFCQRLFMILWLKGVKFNVTTVDMTRKPEELKDLAPGTNPPFLVYNKELKTDFIKIEEFLEQTLAPPRYPHLSPKYKESFDVGCNLFAKFSAYIKNTQKEANKNFEKSLLKEFKRLDDYLNTPLLDEIDPDSAGEPPVSRRLFLDGDQLTLADCSLLPKLNIIKVAAKKYRDFDIPAEFSGVWRYLHNAYAREEFTHTCPEDKEIENTYANVA

SEQ ID NO: 9 - protein, 241 aa, Homo sapiens
MAEEQPQVELFVKAGSDGAKIGNCPFSQRLFMVLWLKGVTFNVTTVDTKRRTETVQKLCPGGQLPFLLYGTEVHTDTNKIEEFLEAVLCPPRYPKLAALNPESNTAGLDIFAKFSAYIKNSNPALNDNLEKGLLKALKVLDNYLTSPLPEEVDETSAEDEGVSQRKFLDGNELTLADCNLLPKLHIVQVVCKKYRGFTIPEAFRGVHRYLSNAYAREEFASTCPDDEEIELAYEQVAKALK

SEQ ID NO: 10 - protein, 253 aa, Rattus norvegicus
MALSMPLNGLKEEDKEPLIELFVKAGSDGESIGNCPFSQRLFMILWLKGVVFSVTTVDLKRKPAHLQNLAPGTHPPFITFNSEVKTDVNKIEEFLEEVLCPPKYLKLSPKHPESNTAGMDIFAKFSAYIKNSRPEANEALERGLLKTLQKLDEYLNSPLPGEIDENSMEDIKSSTRRFLDGDEMTLADCNLLPKLHIVKVVAKKYRNFDIPKGMTGIWRYLTNAYSRDEFTNTCPSDKEVEIAYSDVAKRLTK

SEQ ID NO: 11 - protein, 207 aa, Homo sapiens
MVLLLKGVPFTLTTVDTRRSPDVLKDFAPGSQLPILLYDSDAKTDTLQIEDFLEETLGPPDFPSLAPRYRESNTAGNDVFHKFSAFIKNPVPAQDEALYQQLLRALARLDSYLRAPLEHELAGEPQLRESRRRFLDGDRLTLADCSLLPKLHIVDTVCAHFRQAPIPAELRGVRRYLDSAMQEKEFKYTCPHSAEILAAYRPAVHPR

SEQ ID NO: 12 - DNA, 78 bp, Homo sapiens
aagaactcctcgatcttattcacatccgtcttgacttcaccatcaaaagtcatgaaaggagggtttgttccgggagcc

SEQ ID NO: 13 - DNA, 20 bp, Homo sapiens
gagaaattagctcccccgag

SEQ ID NO: 14 - DNA, 20 bp, Homo sapiens
gcttgggtaagaggttgcag

SEQ ID NO: 15 - DNA, 18 bp, Homo sapiens
atttaggtgacactatag

SEQ ID NO: 16 - DNA, 2061 bp, Homo sapiens
  CDS (1)..(2058)
atggccgaggccgcggagccggagggggttgccccgggtccccaggggccgccggaggtccccgcgcctctggctgagagacccggagagccaggagccgcgggcggggaggcagaagggccggaggggagcgagggcgcagaggaggcgccgaggggcgccgccgctgtgaaggaggcaggaggcggcgggccagacaggggcccggaggccgaggcgcggggcacgaggggggcgcacggcgagactgaggccgaggagggagccccggagggtgccgaggtgccccagggaggggaggagacaagcggcgcgcagcaggtggagggggcgagcccgggacgcggcgcgcagggcgagccccgcggggaggctcagagggagcccgaggactctgcggcccccgagaggcaggaggaggcggagcagaggcctgaggtcccggaaggtagcgcgtccggggaggcgggggacagcgtagacgcggagggcccgctgggggacaacatagaggccgagggcccggcgggcgacagcgtagaggcggagggccgggtgggggacagcgtagacgcggaaggtccggcgggggacagcgtagacgcggagggcccgctgggggacaacatacaagccgagggcccggcgggggacagcgtagacgcggagggccgggtgggggacagcgtagacgcggaaggtccggcgggggacagcgtagacgcggagggccgggtgggggacagcgtagaggcgggggacccggcgggggacggcgtagaagcgggggtcccggcgggggacagcgtagaagccgaaggcccggcgggggacagcatggacgccgagggtccggcaggaagggcgcgccgggtctcgggtgagccgcagcaatcgggggacggcagcctctcgccccaggccgaggcaattgaggtcgcagccggggagagtgcggggcgcagccccggtgagctcgcctgggacgcagcggaggaggcggaggtcccgggggtaaaggggtccgaagaagcggcccccggggacgcaagggcagacgctggcgaggacagggtaggggatgggccacagcaggagccgggggaggacgaagagagacgagagcggagcccggaggggccaagggaggaggaagcagcggggggcgaagaggaatcccccgacagcagcccacatggggaggcctccaggggcgccgcggagcctgaggcccagctcagcaaccacctggccgaggagggccccgccgagggtagcggcgaggccgcgcgcgtgaacggccgccgggaggacggagaggcgtccgagccccgggccctggggcgggagcacgacatcaccctcttcgtcaaggctggttatgatggtgagagtatcggaaattgcccgttttctcagcgtctctttatgattctctggctgaaaggcgttatatttaatgtgaccacagtggacctgaaaaggaaacccgcagacctgcagaacctggctcccggaacaaaccctcctttcatgacttttgatggtgaagtcaagacggatgtgaataagatcgaggagttcttagaggagaaattagctcccccgaggtatcccaagctggggacccaacatcccgaatctaattccgcaggaaatgacgtgtttgccaaattctcagcgtttataaaaaacacgaagaaggatgcaaatgagattcatgaaaagaacctgctgaaggccctgaggaagctggataattacttaaatagccctctgcctgatgaaatagatgcctacagcaccgaggatgtcactgtttctggaaggaagtttctggatggggacgagctgacgctggctgactgcaacctcttacccaagctccatattattaagattgtggccaagaagtacagagattttgaatttccttctgaaatgactggcatctggagatacttgaataatgcttatgctagagatgagttcacaaatacgtgtccagctgatcaagagattgaacacgcatattcagatgttgcaaaaagaatgaaatga

SEQ ID NO: 17 - protein, 686 aa, Homo sapiens
MAEAAEPEGVAPGPQGPPEVPAPLAERPGEPGAAGGEAEGPEGSEGAEEAPRGAAAVKEAGGGGPDRGPEAEARGTRGAHGETEAEEGAPEGAEVPQGGEETSGAQQVEGASPGRGAQGEPRGEAQREPEDSAAPERQEEAEQRPEVPEGSASGEAGDSVDAEGPLGDNIEAEGPAGDSVEAEGRVGDSVDAEGPAGDSVDAEGPLGDNIQAEGPAGDSVDAEGRVGDSVDAEGPAGDSVDAEGRVGDSVEAGDPAGDGVEAGVPAGDSVEAEGPAGDSMDAEGPAGRARRVSGEPQQSGDGSLSPQAEAIEVAAGESAGRSPGELAWDAAEEAEVPGVKGSEEAAPGDARADAGEDRVGDGPQQEPGEDEERRERSPEGPREEEAAGGEEESPDSSPHGEASRGAAEPEAQLSNHLAEEGPAEGSGEAARVNGRREDGEASEPRALGREHDITLFVKAGYDGESIGNCPFSQRLFMILWLKGVIFNVTTVDLKRKPADLQNLAPGTNPPFMTFDGEVKTDVNKIEEFLEEKLAPPRYPKLGTQHPESNSAGNDVFAKFSAFIKNTKKDANEIHEKNLLKALRKLDNYLNSPLPDEIDAYSTEDVTVSGRKFLDGDELTLADCNLLPKLHIIKIVAKKYRDFEFPSEMTGIWRYLNNAYARDEFTNTCPADQEIEHAYSDVAKRMK

SEQ ID NO: 18 - DNA, 21 bp, Homo sapiens
ttctggctgatctgtggcttt

SEQ ID NO: 19 - DNA, 20 bp, Homo sapiens
gcactcagcccacacacaaa

SEQ ID NO: 20 - DNA, 28 bp, Homo sapiens
cctccaccatccctaaccaacctctcat